(19) United States
(12) Patent Application Publication
(10) Pub. No.: US 2015/0361446 A1
(43) Pub. Date: Dec. 17, 2015

(54) MAIZE EVENT DP-033121-3 AND METHODS FOR DETECTION THEREOF

(71) Applicants: Mary BEATTY, Earlham, IA (US); Kent BRINK, Newark, DE (US); Virginia CRANE, Des Moines, IA (US); Scott DIEHN, West Des Moines, IA (US); Albert L. LU, West Des Moines, IA (US); Gregory J. YOUNG, Wilmington, DE (US); PIONEER HI-BRED INTERNATIONAL, INC., Johnston, IA (US); E. I. DUPONT DE NEMOURS & COMPANY, Wilmington, DE (US)

(72) Inventors: Mary Beatty, EARLHAM, IA (US); Kent BRINK, Newark, DE (US); Virginia CRANE, DES MOINES, IA (US); Scott DIEHN, West des Moines, IA (US); Albert L. LU, West des Moines, IA (US); Gregory J. YOUNG, Wilmington, DE (US)

(73) Assignee: Pioneer-Hi-Bred International and E.I. Dupont De Nemours & Company

(21) Appl. No.: 14/763,239

(22) PCT Filed: Jan. 23, 2014
(86) PCT No.: PCT/US2014/012787

§ 371 Date: Jul. 24, 2015

Related U.S. Application Data
(60) Provisional application No. 61/756,874, filed on Jan. 25, 2013.
(60) Provisional application No. 61/843,802, filed on Jul. 8, 2013.

(51) Int. Cl. C12N 15/82; C12Q 1/68; A01H 5/10

(52) U.S. Cl. CPC C12N 15/8286; A01H 5/10; C12Q 1/6895; C12Q 2600/13

(57) ABSTRACT

The disclosure provides DNA compositions that relate to transgenic insect resistant maize plants. Also provided are assays for detecting the presence of the maize DP-033121-3 event based on the DNA sequence of the recombinant construct inserted into the maize genome and the DNA sequences flanking the insertion site. Kits and conditions useful in conducting the assays are provided.

### REFERENCE TO SEQUENCE LISTING SUBMITTED ELECTRONICALLY

A sequence listing having the file name “5648PCT_SeqList.txt” created on Jan. 22, 2014, and having a size of 92 kilobytes is filed in computer readable form concurrently with the specification. The sequence listing is part of the specification and is herein incorporated by reference in its entirety.

### FIELD

Embodiments of the present disclosure relate to the field of plant molecular biology, specifically embodiment of the disclosure relate to DNA constructs for conferring insect resistance to a plant. Embodiments of the disclosure more specifically relate to insect resistant corn plant event DP-033121-3 and to assays for detecting the presence of corn event DP-033121-3 in a sample and compositions thereof.

### BACKGROUND

Corn is an important crop and is a primary food source in many areas of the world. Damage caused by insect pests is a major factor in the loss of the world's corn crops, despite the use of protective measures such as chemical pesticides. In view of this insect resistance, via heterologous genes, has been introduced into crops such as corn in order to control insect damage and to reduce the need for traditional chemical pesticides.

The expression of heterologous genes in plants is known to be influenced by their location in the plant genome and will influence the overall phenotype of the plant in diverse ways. For this reason, it is common to produce hundreds to thousands of different events and screen those events for a single event that has desired transgene expression levels, patterns, and agronomic performance sufficient for commercial purposes. An event that has desired levels or patterns of transgene expression can be used for introgressing the transgene into other genetic backgrounds by sexual outcrossing using conventional breeding methods. Progeny of such crosses maintain the transgene expression characteristics of the original transformant. This strategy is used to ensure reliable gene expression in a number of varieties that are well adapted to local growing conditions.

It would be advantageous to be able to detect the presence of a particular event in order to determine whether progeny of a sexual cross contains an event of interest. In addition, a method for detecting a particular event would be helpful for complying with regulations requiring the pre-market approval and labeling of foods derived from recombinant crop plants, or for use in environmental monitoring, monitoring traits in crops in the field, or monitoring products derived from a crop harvest, as well as for use in ensuring compliance of parties subject to regulatory or contractual terms.

Therefore, a reliable, accurate, method of detecting transgenic event DP-033121-3 is needed.

### SUMMARY

Embodiments of this disclosure relate to methods for producing and selecting an insect resistant monocot crop plant. More specifically, a DNA construct is provided that when expressed in plant cells and plants confers resistance to insects. According to one aspect of the disclosure, a DNA construct, capable of introduction into and replication in a host cell, is provided that when expressed in plant cells and plants confers insect resistance to the plant cells and plants. Maize event DP-033121-3 was produced by Agrobacterium-mediated transformation with plasmid PHP36676. This event contains a cry2A.127, cry1A.88, Vip3Aa20, and mo-pat gene cassettes, which confer resistance to certain lepidopteran and coleopteran pests, as well as tolerance to phosphinothricin.

Specifically, the first cassette contains the cry2A.127 gene encoding the Cry2A.127 protein that has been functionally optimized using DNA shuffling techniques and based on genes derived from Bacillus thuringiensis subsp. kurstaki. The 634-residue protein produced by expression of the cry2A.127 sequence is targeted to maize chloroplasts through the addition of a 54-amino acid chloroplast transit peptide (CTP) (U.S. Pat. No. 7,563,863 B2) as well as a 6-amino acid linker (Peptide Linker) resulting in a total length of 694 amino acids (approximately 77 kDa) for the precursor protein (the CTP sequence is cleaved upon insertion into the chloroplast), resulting in a mature protein of 644 amino acids in length with an approximate molecular weight of 72 kDa; (SEQ ID NO: 17). The expression of the cry2A.127 gene and the CTP is controlled by the promoter from the Citrus Yellow Mosaic Virus (CYMV) (Huang and Hartung, 2001, Journal of General Virology 82: 2549-2558; Genbank accession NC_{—}003382.1) along with the intron 1 region from maize alcohol dehydrogenase gene (Adh1 Intron) (Dennis et al., 1984, Nucleic Acids Research 12: 3983-4000). Transcription of the cry2A.127 gene cassette is terminated by the presence of the terminator from the ubiquitin 3 (UBQ3) gene of Arabidopsis thaliana (Callis et al., 1995, Genetics 139: 921-939). In addition, a genomic fragment corresponding to the 3′ untranslated region from a ribosomal protein gene (RPG 3′ UTR) of Arabidopsis thaliana (Salanoubat et al., 2000, Nature 408: 820-822; TAIR accession AT3G28500) is located between the cry2A.127 and cry1A.88 cassettes in order to prevent any potential transcriptional interference with downstream cassettes. Transcriptional interference is defined as the transcriptional suppression of one gene on another when both are in close proximity (Shearwin, et al., 2005, Trends in Genetics 21: 339-345). The presence of a transcriptional terminator between two cassettes has been shown to reduce the occurrence of transcriptional interference (Greger et al., 1998, Nucleic Acids Research 26: 1294-1300); the placement of multiple terminators between cassettes is intended to prevent this effect.

The second cassette (cry1A.88 gene cassette) contains a second shuffled insect control gene, cry1A.88, encoding the Cry1A.88 protein that has been functionally optimized using DNA shuffling techniques and based on genes derived from Bacillus thuringiensis subsp. kurstaki. The coding region which produces a 1,182-residue protein (approximately 134 kDa; SEQ ID NO: 18) is controlled by a truncated version of the promoter from Banana Streak Virus of acuminata Vietnam strain [BSV (AV)] (Lheureux et al., 2007, Archives of Virology 152: 1409-1416; Genbank accession NC_{—}007003.1) with a second copy of the maize Adh1 intron. The terminator for the cry1A.88 cassette is a portion of the Sorghum bicolor genome containing the terminator from the actin gene (SB-actin) (Genbank accession XM_{—}002441128.1).

The third cassette (vip3Aa20 gene cassette) contains the modified vip3A gene derived from Bacillus thuringiensis strain AB88, which encodes the insecticidal Vip3Aa20 protein (Estruch et al., 1996, PNAS 93: 5389-5394). Expression of the vip3Aa20 gene is controlled by the regulatory region of the maize polyubiquitin (ubiZM1) gene, including the promoter, the 5′ untranslated region (5′ UTR) and intron (Christensen et al., 1992, Plant Molecular Biology 18: 675-689). The terminator for the vip3Aa20 gene is the terminator sequence from the proteinase inhibitor II (pinII) gene of Solanum tuberosum (Keil et al., 1986, Nucleic Acids Research 14: 5641-5650; An et al., 1989, The Plant Cell 1: 115-122). The Vip3Aa20 protein is 789-amino acid residues in length with an approximate molecular weight of 88 kDa (SEQ ID NO: 19).

The fourth gene cassette (mo-pat gene cassette) contains a maize-optimized version of the phosphinothricin acetyl transferase gene (mo-pat) from Streptomyces viridochromogenes (Wohlleben et al., 1988, Gene 70: 25-37). The mo-pat gene expresses the phosphinothricin acetyl transferase (PAT) enzyme that confers tolerance to phosphinothricin. The PAT protein is 183 amino acids in length and has an approximate molecular weight of 21 kDa (SEQ ID NO: 20). Expression of the mo-pat gene is controlled by a second copy of the ubiZM1 promoter, the 5′ UTR and intron (Christensen et al., 1992, Plant Molecular Biology 18: 675-689), in conjunction with a second copy of the pinII terminator (Keil et al., 1986, Nucleic Acids Research 14: 5641-5650; An et al., 1989, The Plant Cell 1: 115-122).

According to another embodiment of the disclosure, compositions and methods are provided for identifying a novel corn plant designated DP-033121-3. The methods are based on primers or probes which specifically recognize the 5′ and/or 3′ flanking sequence of DP-033121-3. DNA molecules are provided that comprise primer sequences that when utilized in a PCR reaction will produce amplicons unique to the transgenic event DP-033121-3. The corn plant and seed comprising these molecules is an embodiment of this disclosure. Further, kits utilizing these primer sequences for the identification of the DP-033121-3 event are provided.

An additional embodiment of the disclosure relates to the specific flanking sequence of DP-033121-3 described herein, which can be used to develop specific identification methods for DP-033121-3 in biological samples. More particularly, the disclosure relates to the 5′ and/or 3′ flanking regions of DP-033121-3 which can be used for the development of specific primers and probes. A further embodiment of the disclosure relates to identification methods for the presence of DP-033121-3 in biological samples based on the use of such specific primers or probes.

According to another embodiment of the disclosure, methods of detecting the presence of DNA corresponding to the corn event DP-033121-3 in a sample are provided. Such methods comprise: (a) contacting the sample comprising DNA with a DNA primer set, that when used in a nucleic acid amplification reaction with genomic DNA extracted from corn event DP-033121-3 produces an amplicon that is diagnostic for corn event DP-033121-3; (b) performing a nucleic acid amplification reaction, thereby producing the amplicon; and (c) detecting the amplicon.

According to another embodiment of the disclosure, methods of detecting the presence of a DNA molecule corresponding to the DP-033121-3 event in a sample, such methods comprising: (a) contacting the sample comprising DNA extracted from a corn plant with a DNA probe molecule that hybridizes under stringent hybridization conditions with DNA extracted from corn event DP-033121-3 and does not hybridize under the stringent hybridization conditions with a control corn plant DNA; (b) subjecting the sample and probe to stringent hybridization conditions; and (c) detecting hybridization of the probe to the DNA. More specifically, a method for detecting the presence of a DNA molecule corresponding to the DP-033121-3 event in a sample, such methods, consisting of (a) contacting the sample comprising DNA extracted from a corn plant with a DNA probe molecule that consists of sequences that are unique to the event, e.g. junction sequences, wherein said DNA probe molecule hybridizes under stringent hybridization conditions with DNA extracted from corn event DP-033121-3 and does not hybridize under the stringent hybridization conditions with a control corn plant DNA; (b) subjecting the sample and probe to stringent hybridization conditions; and (c) detecting hybridization of the probe to the DNA.

In addition, a kit and methods for identifying event DP-033121-3 in a biological sample which detects a DP-033121-3 specific region are provided.

DNA molecules are provided that comprise at least one junction sequence of DP-033121-3; wherein a junction sequence spans the junction between heterologous DNA inserted into the genome and the DNA from the corn cell flanking the insertion site, i.e. flanking DNA, and is diagnostic for the DP-033121-3 event.

According to another embodiment of the disclosure, methods of producing an insect resistant corn plant that comprise the steps of: (a) sexually crossing a first parental corn line comprising the expression cassettes of the disclosure, which confers resistance to insects, and a second parental corn line that lacks insect resistance, thereby producing a plurality of progeny plants; and (b) selecting a progeny plant that is insect resistant. Such methods may optionally comprise the further step of back-crossing the progeny plant to the second parental corn line to producing a true-breeding corn plant that is insect resistant.

A further embodiment of the disclosure provides a method of producing a corn plant that is resistant to insects comprising transforming a corn cell with the DNA construct PHP36676, growing the transformed corn cell into a corn plant, selecting the corn plant that shows resistance to insects, and further growing the corn plant into a fertile corn plant. The fertile corn plant can be self-pollinated or crossed with compatible corn varieties to produce insect resistant progeny. In some embodiments the event DP-033121-3 was generated by transforming the maize line PHWWE with plasmid PHP36676.

Another embodiment of the disclosure further relates to a DNA detection kit for identifying maize event DP-033121-3 in biological samples. The kit comprises a first primer which specifically recognizes the 5′ or 3′ flanking region of DP-033121-3, and a second primer which specifically recognizes a sequence within the foreign DNA of DP-033121-3, or within the flanking DNA, for use in a PCR identification protocol. A further embodiment of the disclosure relates to a kit for identifying event DP-033121-3 in biological samples, which kit comprises a specific probe having a sequence which corresponds or is complementary to, a sequence having between 80% and 100% sequence identity with a specific region of event DP-033121-3. The sequence of the probe corresponds to a specific region comprising part of the 5′ or 3′ flanking region of event DP-033121-3.

The methods and kits encompassed by the embodiments of the present disclosure can be used for different purposes such as, but not limited to the following: to identify event DP-033121-3 in plants, plant material or in products such as, but not limited to, food or feed products (fresh or processed) comprising, or derived from plant material; additionally or alternatively, the methods and kits can be used to identify transgenic plant material for purposes of segregation between transgenic and non-transgenic material; additionally or alternatively, the methods and kits can be used to determine the quality of plant material comprising maize event DP-033121-3. The kits may also contain the reagents and materials necessary for the performance of the detection method.

A further embodiment of this disclosure relates to the DP-033121-3 corn plant or its parts, including, but not limited to, pollen, ovules, vegetative cells, the nuclei of pollen cells, and the nuclei of egg cells of the corn plant DP-033121-3 and the progeny derived thereof. The corn plant and seed of DP-033121-3 from which the DNA primer molecules provide a specific amplicon product is an embodiment of the disclosure.

The following embodiments are encompassed by the present disclosure.

1. A DNA construct comprising:
(a) a first expression cassette, comprising in operable linkage:

(i) a full length Citrus Yellow Mosaic virus (CYMV) promoter;

(ii) a maize adh1 first intron;

(iii) a synthetic chloroplast targeting peptide

(iv) a Cry2A.127 encoding DNA molecule;

(v) a ubiquitin3 (UBQ3) transcriptional terminator; and

(vi) a 3′ untranslated region of an Arabidopsis ribosomal protein gene;

(b) a second expression cassette, comprising in operable linkage:

(i) a truncated BSV promoter and second adh1 intron;

(ii) a Cry1A.88 encoding DNA molecule; and

(iii) a sorghum actin transcriptional terminator;

(c) a third expression cassette, comprising in operable linkage:

(i) a maize polyubiquitin promoter;

(ii) a 5′ untranslated region and intron1 of a maize polyubiquitin gene;

(iii) a Vip3Aa20 encoding DNA molecule; and

(iv) a pinII transcriptional terminator; and

(d) a fourth expression cassette comprising in operable linkage:

(i) a maize polyubiquitin promoter;

(ii) a mo-pat encoding DNA molecule; and

(ii) a pinII transcriptional terminator.

2. The DNA construct of embodiment 1, comprising the sequence of SEQ ID NO: 1.
3. The DNA construct of embodiment 1, wherein the DNA construct is flanked by the 5′ junction sequence of SEQ ID NO: 15 and the 3′ junction sequence of SEQ ID NO: 16.
4. A plant transformed with the DNA construct of embodiment 1 or 2.
5. A corn plant, comprising the sequence set forth in SEQ ID NO: 14.
6. A corn plant comprising event DP-033121-3, wherein a representative sample of seed of said corn event has been deposited with American Type Culture Collection (ATCC) with Accession No. PTA-13392.
7. Plant parts of the corn event of embodiment 6.
8. Seed comprising corn event DP-033121-3, wherein said seed comprises a DNA molecule having nucleic acid sequence of SEQ ID NO: 14.
8. Progeny of the corn plant of claim 4, 5, 6, or 7, or part thereof, wherein the progeny comprises a polynucleotide having a sequence of SEQ ID NO: 14.
9. A transgenic seed produced from the corn plant of embodiment 8 comprising event DP-033121-3.
10. A transgenic corn plant, or part thereof, grown from the seed of embodiment 9.
11. An isolated nucleic acid molecule comprising a nucleotide sequence selected from the group consisting of SEQ ID NO: 9; SEQ ID NO: 14; SEQ ID NO: 8, and full length complements thereof.
12. An amplicon comprising the nucleic acid sequence selected from the group consisting of SEQ ID NO: 9, and full length complements thereof.
13. A biological sample derived from corn event DP-033121-3 plant, tissue, or seed, wherein said sample comprises a nucleotide sequence selected from the group consisting of SEQ ID NO: 9, SEQ ID NO: 14, SEQ ID NO: 8 and the complement thereof, wherein said nucleotide sequence is detectable in said sample using a nucleic acid amplification or nucleic acid hybridization method, wherein a representative sample of said corn event DP-033121-3 seed of has been deposited with American Type Culture Collection (ATCC) with Accession No. PTA-13392.
14. The biological sample of embodiment 13, wherein said biological sample comprise plant, tissue, or seed of transgenic corn event DP-033121-3.
15. The biological sample of embodiment 14, wherein said biological sample is a DNA sample extracted from the transgenic corn plant event DP-033121-3, and wherein said DNA sample comprises one or more of the nucleotide sequences selected from the group consisting of SEQ ID NO: 9, SEQ ID NO: 14, SEQ ID NO: 8, and the complement thereof.
16. The biological sample of embodiment 15, wherein said biological sample is selected from the group consisting of corn flour, corn meal, corn syrup, and cereals manufactured in whole or in part to contain corn by-products.

### 17. A Method for Producing a Corn Plant Resistant to Lepidopteran Pests, Comprising:

(a) sexually crossing a first parent corn plant with a second parent corn plant, wherein said first or second parent corn plant comprises event DP-033121-3 DNA, thereby producing a plurality of first generation progeny plants;
(b) selecting a first generation progeny plant that is resistant to lepidopteran insect infestation;
(c) selfing the first generation progeny plant, thereby producing a plurality of second generation progeny plants; and
(d) selecting from the second generation progeny plants, a plant that is resistant to lepidopteran pests;
wherein the second generation progeny plants comprise the DNA construct according to embodiment 1.
   18. A method of producing hybrid corn seeds comprising:
(a) planting seeds of a first inbred corn line comprising the DNA construct of embodiment 1 and seeds of a second inbred line having a genotype different from the first inbred corn line;
(b) cultivating corn plants resulting from said planting until time of flowering;
(c) emasculating said flowers of plants of one of the corn inbred lines;
(d) sexually crossing the two different inbred lines with each other; and
(e) harvesting the hybrid seed produced thereby.
   19. The method of embodiment 18 further comprising the step of backcrossing the second generation progeny plant of step (d) that comprises corn event DP-033121-3 DNA to the parent plant that lacks the corn event DP-033121-3 DNA, thereby producing a backcross progeny plant that is resistant to at least lepidopteran insects.
   20. A method for producing a corn plant resistant to at least lepidopteran insects, said method comprising:
(a) sexually crossing a first parent corn plant with a second parent corn plant, wherein said first or second parent corn plant is a corn event DP-033121-3 plant, thereby producing a plurality of first generation progeny plants;
(b) selecting a first generation progeny plant that is resistant to at least lepidopteran insect infestation;
(c) backcrossing the first generation progeny plant of step (b) with the parent plant that lacks corn event DP-033121-3 DNA, thereby producing a plurality of backcross progeny plants; and
(d) selecting from the backcross progeny plants, a plant that is resistant to at least lepidopteran insect infestation;
wherein the selected backcross progeny plant of step (d) comprises SEQ ID NO:14.
   21. The method according to embodiment 20, wherein the plants of the first inbred corn line are the female parents or male parents.
   22. Hybrid seed produced by the method of embodiment 21.
   23. A method of detecting the presence of a nucleic acid molecule that is unique to event DP-033121-3 in a sample comprising corn nucleic acids, the method comprising:
(a) contacting the sample with a pair of primers that, when used in a nucleic-acid amplification reaction with genomic DNA from event DP-033121-3 produces an amplicon that is diagnostic for event DP-033121-3;
(b) performing a nucleic acid amplification reaction, thereby producing the amplicon; and
(c) detecting the amplicon.
   23. A pair of polynucleotide primers comprising a first polynucleotide primer and a second polynucleotide primer which function together in the presence of event DP-033121-3 DNA template in a sample to produce an amplicon diagnostic for event DP-033121-3.
   24. The pair of polynucleotide primers according to embodiment 23, wherein the sequence of the first polynucleotide primer is or is complementary to a corn plant genome sequence flanking the point of insertion of a heterologous DNA sequence inserted into the corn plant genome of event DP-033121-3, and the sequence of the second polynucleotide primer is or is complementary to the heterologous DNA sequence inserted into the genome of event DP-033121-3.
   25. A method of detecting the presence of DNA corresponding to the DP-033121-3 event in a sample, the method comprising:
(a) contacting the sample comprising maize DNA with a polynucleotide probe that hybridizes under stringent hybridization conditions with DNA from maize event DP-033121-3 and does not hybridize under said stringent hybridization conditions with a non-DP-033121-3 maize plant DNA;
(b) subjecting the sample and probe to stringent hybridization conditions; and
(c) detecting hybridization of the probe to the DNA;
wherein detection of hybridization indicates the presence of the DP-033121-3 event.
   26. A kit for detecting nucleic acids that are unique to event DP-033121-3 comprising at least one nucleic acid molecule of sufficient length of contiguous polynucleotides to function as a primer or probe in a nucleic acid detection method, and which upon amplification of or hybridization to a target nucleic acid sequence in a sample followed by detection of the amplicon or hybridization to the target sequence, are diagnostic for the presence of nucleic acid sequences unique to event DP-033121-3 in the sample.
   27. The kit according to embodiment 26, wherein the nucleic acid molecule comprises a nucleotide sequence selected from the group consisting of SEQ ID NO: 8 and SEQ ID NO: 9.

The foregoing and other aspects of the disclosure will become more apparent from the following detailed description and accompanying drawing.

### BRIEF DESCRIPTION OF THE DRAWINGS

FIG. 1 shows a schematic diagram of plasmid PHP36676 with genetic elements indicated.

FIG. 2 shows a schematic diagram of the T-DNA region from plasmid PHP36676 with the identification of the cry2A.127, cry1A.88, vip3Aa20, and mo-pat gene cassettes. The size of the T-DNA is 24,266 base pairs.

### DETAILED DESCRIPTION

The disclosure relates to the insect resistant corn (Zea mays) plant DP-033121-3, also referred to as “maize line DP-033121-3,” “maize event DP-033121-3,” and “033121 maize,” and to the DNA plant expression construct of corn plant DP-033121-3 and the detection of the transgene/flanking insertion region in corn plant DP-033121-3 and progeny thereof.

According to one embodiment, compositions and methods are provided for identifying a novel corn plant designated DP-033121-3. The methods are based on primers or probes which specifically recognize the 5′ and/or 3′ flanking sequence of DP-033121-3. DNA molecules are provided that comprise primer sequences that when utilized in a PCR reaction will produce amplicons unique to the transgenic event DP-033121-3. The corn plant and seed comprising these molecules is an embodiment of this disclosure. Further, kits utilizing these primer sequences for the identification of the DP-033121-3 event are provided.

An additional embodiment relates to the specific flanking sequence of DP-033121-3 described herein, which can be used to develop specific identification methods for DP-033121-3 in biological samples. Some embodiments relate to the 5′ and/or 3′ flanking regions of DP-033121-3 which can be used for the development of specific primers and probes. A further embodiment relates to identification methods for the presence of DP-033121-3 in biological samples based on the use of such specific primers or probes.

According to another embodiment, methods of detecting the presence of DNA corresponding to the corn event DP-033121-3 in a sample are provided. Such methods comprise: (a) contacting the sample comprising DNA with a DNA primer set, that when used in a nucleic acid amplification reaction with genomic DNA extracted from corn event DP-033121-3 produces an amplicon that is diagnostic for corn event DP-033121-3; (b) performing a nucleic acid amplification reaction, thereby producing the amplicon; and (c) detecting the amplicon.

According to another embodiment, methods of detecting the presence of a DNA molecule corresponding to the DP-033121-3 event in a sample, such methods comprising: (a) contacting the sample comprising DNA extracted from a corn plant with a DNA probe molecule that hybridizes under stringent hybridization conditions with DNA extracted from corn event DP-033121-3 and does not hybridize under the stringent hybridization conditions with a control corn plant DNA; (b) subjecting the sample and probe to stringent hybridization conditions; and (c) detecting hybridization of the probe to the DNA. More specifically, a method for detecting the presence of a DNA molecule corresponding to the DP-033121-3 event in a sample, such methods, consisting of (a) contacting the sample comprising DNA extracted from a corn plant with a DNA probe molecule that consists of sequences that are unique to the event, e.g. junction sequences, wherein said DNA probe molecule hybridizes under stringent hybridization conditions with DNA extracted from corn event DP-033121-3 and does not hybridize under the stringent hybridization conditions with a control corn plant DNA; (b) subjecting the sample and probe to stringent hybridization conditions; and (c) detecting hybridization of the probe to the DNA.

In addition, a kit and methods for identifying event DP-033121-3 in a biological sample which detects a DP-033121-3 specific region are provided.

DNA molecules are provided that comprise at least one junction sequence of DP-033121-3; wherein a junction sequence spans the junction between heterologous DNA inserted into the genome and the DNA from the corn cell flanking the insertion site, i.e. flanking DNA, and is diagnostic for the DP-033121-3 event.

According to another embodiment, methods of producing an insect resistant corn plant that comprise the steps of: (a) sexually crossing a first parental corn line comprising the expression cassettes, which confers resistance to insects, and a second parental corn line that lacks insect resistance, thereby producing a plurality of progeny plants; and (b) selecting a progeny plant that is insect resistant. Such methods may optionally comprise the further step of back-crossing the progeny plant to the second parental corn line to producing a true-breeding corn plant that is insect resistant.

A further embodiment provides a method of producing a corn plant that is resistant to insects comprising transforming a corn cell with the DNA construct PHP36676, growing the transformed corn cell into a corn plant, selecting the corn plant that shows resistance to insects, and further growing the corn plant into a fertile corn plant. The fertile corn plant can be self-pollinated or crossed with compatible corn varieties to produce insect resistant progeny.

Another embodiment further relates to a DNA detection kit for identifying maize event DP-033121-3 in biological samples. The kit comprises a first primer which specifically recognizes the 5′ or 3′ flanking region of DP-033121-3, and a second primer which specifically recognizes a sequence within the foreign DNA of DP-033121-3, or within the flanking DNA, for use in a PCR identification protocol. A further embodiment relates to a kit for identifying event DP-033121-3 in biological samples, which kit comprises a specific probe having a sequence which corresponds or is complementary to, a sequence having between 80% and 100% sequence identity with a specific region of event DP-033121-3. The sequence of the probe corresponds to a specific region comprising part of the 5′ or 3′ flanking region of event DP-033121-3.

The methods and kits encompassed by the embodiments can be used for different purposes such as, but not limited to the following: to identify event DP-033121-3 in plants, plant material or in products such as, but not limited to, food or feed products (fresh or processed) comprising, or derived from plant material; additionally or alternatively, the methods and kits can be used to identify transgenic plant material for purposes of segregation between transgenic and non-transgenic material; additionally or alternatively, the methods and kits can be used to determine the quality of plant material comprising maize event DP-033121-3. The kits may also contain the reagents and materials necessary for the performance of the detection method.

A further embodiment relates to the DP-033121-3 corn plant or its parts, including, but not limited to, pollen, ovules, vegetative cells, the nuclei of pollen cells, and the nuclei of egg cells of the corn plant DP-033121-3 and the progeny derived thereof. The corn plant and seed of DP-033121-3 from which the DNA primer molecules provide a specific amplicon product is an embodiment of the disclosure.

Specifically, the first cassette contains the proprietary cry2A.127 gene, a Cry2Ab-like coding sequence that has been functionally optimized using DNA shuffling and directed mutagenesis techniques. The 634 residue protein produced by expression of the cry2A.127 sequence is targeted to maize chloroplasts through the addition of a 56 amino acid codon-optimized synthetic chloroplast targeting peptide (CTP) as well as 4 synthetic linker amino acids, resulting in a total length of 694 amino acids (approximately 77 kDa) for the precursor protein (the Cry2A.127 CTP sequence is cleaved upon insertion into the chloroplast, resulting in a mature protein of approximately 71 kDa). The expression of the cry2A.127 gene and attached transit peptide is controlled by the full length promoter from the CYMV promoter (Citrus Yellow Mosaic Virus; Genbank accession AF347695.1) along with a downstream copy of the maize adh1 intron (Dennis et al., 1984, Nucleic Acids Research 12: 3983-4000). Transcription of the cry2A.127 gene cassette is terminated by the downstream presence of the Arabidopsis thaliana ubiquitin 3 (UBQ3) termination region (Callis et al., 1995 Genetics 139: 921-939). In addition, a 2.2 kB fragment corresponding to the 3′ un-translated region from an Arabidopsis ribosomal protein gene (TAIR accession AT3G28500; Salanoubat et al., 2000 Nature 408: 820-822) is located between the cry2A.127 and cry1A.88 cassettes in order to eliminate any potential read thru transcripts.

The second cassette contains a second shuffled proprietary insect control gene, the Cry1A-like cry1A.88 coding region. This 1182 residue coding region (which produces a precursor protein of approximately 133 kDa, is controlled by a truncated version (470 nucleotides in length) of the full length promoter from Banana Streak Virus (Acuminate Vietnam strain; Lheureux et al., 2007 Archives of Virology 152: 1409-1416) along with a second copy of the maize adh1 intron. The termination region for the cry1A.88 cassette is a 1.1 kB portion of the Sorghum bi-color genome containing the 3′ termination region from the SB-Actin gene (Genbank accession XM_{—}002441128.1). Three other termination regions are present between the second and third cassettes; the 27 kD gamma zein terminator originally isolated from maize line W64A (Das et al., 1991 Genomics 11: 849-856), a genomic fragment of Arabidopsis thaliana chromosome 4 containing the Ubiquitin-14 (UBQ14) 3′UTR and terminator (Callis et al., 1995 Ecotype Columbia. Genetics 139: 921-939) and the termination sequence from the maize In2-1 gene (Hershey and Stoner, 1991 Plant Molecular Biology 17: 679-690).

The third cassette contains the vip3Aa20 gene, which codes for a synthetic version of the insecticidal Vip3Aa20 protein (present in the approved Syngenta event MIR162; Estruch et al., 1996 PNAS 93: 5389-5394). Expression of the vip3Aa20 gene is controlled by the maize polyubiquitin promoter, including the 5′ untranslated region and intron 1 (Christensen et al., 1992 Plant Molecular Biology 18: 675-689). The terminator for the vip3Aa20 gene is the 3′ terminator sequence from the proteinase inhibitor II gene of Solanum tuberosum (pinII terminator) (Keil et al., 1986, Nucleic Acids Research 14: 5641-5650; An et al., 1989, The Plant Cell 1: 115-122). The Vip3Aa20 protein is 789 amino acid residues in length with an approximate molecular weight of 88 kDa.

The fourth and final gene cassette contains a version of the phosphinothricin acetyl transferase gene (mo-pat) from Streptomyces viridochromogenes (Wohlleben et al., 1988 Gene 70: 25-37) that has been optimized for expression in maize. The pat gene expresses the phosphinothricin acetyl transferase enzyme (PAT) that confers tolerance to phosphinothricin. The PAT protein is 183 amino acids residues in length and has a molecular weight of approximately 21 kDa. Expression of the mo-pat gene is controlled by a second copy of the maize polyubiquitin promoter/5′UTR/intron in conjunction with a second copy of the pinII terminator. Plants containing the DNA constructs are also provided. A description of the genetic elements in the PHP36676 T-DNA (set forth in SEQ ID NO: 1) and their sources are described further in the Table 3.

The following definitions and methods are provided to better define the present disclosure and to guide those of ordinary skill in the art in the practice of the present disclosure. Unless otherwise noted, terms are to be understood according to conventional usage by those of ordinary skill in the relevant art. Definitions of common terms in molecular biology may also be found in Rieger et al., Glossary of Genetics: Classical and Molecular, 5^{th} edition, Springer-Verlag; New York, 1991; and Lewin, Genes V, Oxford University Press: New York, 1994. The nomenclature for DNA bases as set forth at 37 CFR §1.822 is used.

The following table sets forth abbreviations used throughout this document, and in particular in the Examples section.

Table of Abbreviations

| | |
|---|---|
| 033121 maize | Maize containing event DP-033121-3 |
| Bp | Base pair |
| BSV | Banana Streak Virus |
| Bt | Bacillus thuringiensis |
| cry2A.127 | cry2A.127-like coding sequence functionally optimized using DNA |
| | shuffling and directed mutagenesis techniques |
| Cry2A.127 | Protein from cry2A.127 gene |
| cry1A.88 | cry1A.88-like coding sequence (including protoxin regions) functionally |
| | optimized using DMA shuffling and directed mutagenesis techniques |
| Cry1A.88 | Protein from cry1A.88 gene |
| CYMV | Citrus Yellow Mosaic Virus |
| kb | Kilobase pair |
| kDa | KiloDalton |
| LB | Left T-DNA border |
| mo-pat | Maize-optimized version of the phosphinothricin acetyl transferase gene |
| | (pat) from Streptomyces viridochromgenes |
| MO-PAT | Protein from phosphinothricin acetyl transferase gene |
| PCR | Polymerase chain reaction |
| pinII | Proteinase inhibitor II gene from Solarium tuberosum |
| RB | Right T-DNA border |
| T-DNA | The transfer DNA portion of the Agrobacterium transformation plasmid |
| | between the Left and Right Borders that is expected to be transferred to |
| | the plant genome |
| UBQ3 | ubiquitin 3 gene of Arabidopsis thaliana |
| ubiZM1 | Promoter region from Zea mays polyubiquitin gene |
| UTR | Untranslated region |
| vip3Aa20 | Synthetic vip3Aa20 gene (present in approved Syngenta event MIR162) |
| Vip3Aa20 | Protein from vip3Aa20 gene |
| ECB | European corn borer (Ostrinia nubilalis) |
| FAW | Fall armyworm (Spodoptera frugiperda) |
| CEW | Corn earworm |

Compositions of this disclosure include seed deposited as Patent Deposit No. PTA-13392 and plants, plant cells, and seed derived therefrom. Applicant(s) have made a deposit of at least 2500 seeds of maize event DP-033121-3 with the American Type Culture Collection (ATCC), Manassas, Va. 20110-2209 USA, on Dec. 12, 2012 and the deposits were assigned ATCC Deposit No. PTA-13392. These deposits will be maintained under the terms of the Budapest Treaty on the International Recognition of the Deposit of Microorganisms for the Purposes of Patent Procedure. These deposits were made merely as a convenience for those of skill in the art and are not an admission that a deposit is required under 35 U.S.C. §112. The seeds deposited with the ATCC on Dec. 12, 2012 were taken from the deposit maintained by Pioneer Hi-Bred International, Inc., 7250 NW 62^{nd} Avenue, Johnston, Iowa 50131-1000. Access to this deposit will be available during the pendency of the application to the Commissioner of Patents and Trademarks and persons determined by the Commissioner to be entitled thereto upon request. Upon allowance of any claims in the application, the Applicant(s) will make available to the public, pursuant to 37 C.F.R. §1.808, sample(s) of the deposit of at least 2500 seeds of hybrid maize with the American Type Culture Collection (ATCC), 10801 University Boulevard, Manassas, Va. 20110-2209. This deposit of seed of maize event DP-033121-3 will be maintained in the ATCC depository, which is a public depository, for a period of 30 years, or 5 years after the most recent request, or for the enforceable life of the patent, whichever is longer, and will be replaced if it becomes nonviable during that period. Additionally, Applicant(s) have satisfied all the requirements of 37 C.F.R. §§1.801-1.809, including providing an indication of the viability of the sample upon deposit. Applicant(s) have no authority to waive any restrictions imposed by law on the transfer of biological material or its transportation in commerce. Applicant(s) do not waive any infringement of their rights granted under this patent or rights applicable to event DP-033121-3 under the Plant Variety Protection Act (7 USC 2321 et seq.). Unauthorized seed multiplication prohibited. The seed may be regulated.

As used herein, the term “comprising” means “including but not limited to.”

As used herein, the term “corn” means Zea mays or maize and includes all plant varieties that can be bred with corn, including wild maize species.

As used herein, the term “DP-033121-3 specific” refers to a nucleotide sequence which is suitable for discriminatively identifying event DP-033121-3 in plants, plant material, or in products such as, but not limited to, food or feed products (fresh or processed) comprising, or derived from plant material.

As used herein, the terms “insect resistant” and “impacting insect pests” refers to effecting changes in insect feeding, growth, and/or behavior at any stage of development, including but not limited to: killing the insect; retarding growth; preventing reproductive capability; inhibiting feeding; and the like.

As used herein, the terms “pesticidal activity” and “insecticidal activity” are used synonymously to refer to activity of an organism or a substance (such as, for example, a protein) that can be measured by numerous parameters including, but not limited to, pest mortality, pest weight loss, pest attraction, pest repellency, and other behavioral and physical changes of a pest after feeding on and/or exposure to the organism or substance for an appropriate length of time. For example “pesticidal proteins” are proteins that display pesticidal activity by themselves or in combination with other proteins.

“Coding sequence” refers to a nucleotide sequence that codes for a specific amino acid sequence. As used herein, the terms “encoding” or “encoded” when used in the context of a specified nucleic acid mean that the nucleic acid comprises the requisite information to guide translation of the nucleotide sequence into a specified protein. The information by which a protein is encoded is specified by the use of codons. A nucleic acid encoding a protein may comprise non-translated sequences (e.g., introns) within translated regions of the nucleic acid or may lack such intervening non-translated sequences (e.g., as in cDNA).

“Gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” refers any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. “Foreign” refers to material not normally found in the location of interest. Thus “foreign DNA” may comprise both recombinant DNA as well as newly introduced, rearranged DNA of the plant. A “foreign” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure. The site in the plant genome where a recombinant DNA has been inserted may be referred to as the “insertion site” or “target site”.

As used herein, “insert DNA” refers to the heterologous DNA within the expression cassettes used to transform the plant material while “flanking DNA” can exist of either genomic DNA naturally present in an organism such as a plant, or foreign (heterologous) DNA introduced via the transformation process which is extraneous to the original insert DNA molecule, e.g. fragments associated with the transformation event. A “flanking region” or “flanking sequence” as used herein refers to a sequence of at least 20 bp, preferably at least 50 bp, and up to 5000 bp, which is located either immediately upstream of and contiguous with or immediately downstream of and contiguous with the original foreign insert DNA molecule. Transformation procedures leading to random integration of the foreign DNA will result in transformants containing different flanking regions characteristic and unique for each transformant. When recombinant DNA is introduced into a plant through traditional crossing, its flanking regions will generally not be changed. Transformants will also contain unique junctions between a piece of heterologous insert DNA and genomic DNA, or two (2) pieces of genomic DNA, or two (2) pieces of heterologous DNA. A “junction” is a point where two (2) specific DNA fragments join. For example, a junction exists where insert DNA joins flanking DNA. A junction point also exists in a transformed organism where two (2) DNA fragments join together in a manner that is modified from that found in the native organism. “Junction DNA” refers to DNA that comprises a junction point. Two junction sequences set forth in this disclosure are the junction point between the maize genomic DNA and the 5′ end of the insert as set forth in the forward junction sequences and the junction point between the 3′ end of the insert and maize genomic DNA as set forth in the reverse junction sequences.

As used herein, “heterologous” in reference to a nucleic acid is a nucleic acid that originates from a foreign species, or, if from the same species, is substantially modified from its native form in composition and/or genomic locus by deliberate human intervention. For example, a promoter operably linked to a heterologous nucleotide sequence can be from a species different from that from which the nucleotide sequence was derived, or, if from the same species, the promoter is not naturally found operably linked to the nucleotide sequence. A heterologous protein may originate from a foreign species, or, if from the same species, is substantially modified from its original form by deliberate human intervention.

“Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences can include, without limitation: promoters, translation leader sequences, introns, and polyadenylation recognition sequences.

“Promoter” refers to a nucleotide sequence capable of controlling the expression of a coding sequence or functional RNA. In general, a coding sequence is located 3′ to a promoter sequence. The promoter sequence consists of proximal and more distal upstream elements, the latter elements are often referred to as enhancers. Accordingly, an “enhancer” is a nucleotide sequence that can stimulate promoter activity and may be an innate element of the promoter or a heterologous element inserted to enhance the level or tissue-specificity of a promoter. Promoters may be derived in their entirety from a native gene, or be composed of different elements derived from different promoters found in nature, or even comprise synthetic nucleotide segments. It is understood by those skilled in the art that different promoters may direct the expression of a gene in different tissues or cell types, or at different stages of development, or in response to different environmental conditions. Promoters that cause a nucleic acid fragment to be expressed in most cell types at most times are commonly referred to as “constitutive promoters”. New promoters of various types useful in plant cells are constantly being discovered; numerous examples may be found in the compilation by Okamuro and Goldberg (1989) Biochemistry of Plants 15:1-82. It is further recognized that since in most cases the exact boundaries of regulatory sequences have not been completely defined, nucleic acid fragments of different lengths may have identical promoter activity.

The “translation leader sequence” refers to a nucleotide sequence located between the promoter sequence of a gene and the coding sequence. The translation leader sequence is present in the fully processed mRNA upstream of the translation start sequence. The translation leader sequence may affect numerous parameters including, but not limited to, processing of the primary transcript to mRNA, mRNA stability and/or translation efficiency. Examples of translation leader sequences have been described (Turner and Foster (1995) Mol. Biotechnol. 3:225-236).

The “3′ non-coding sequences” refer to nucleotide sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al. (1989) Plant Cell 1:671-680.

A “protein” or “polypeptide” is a chain of amino acids arranged in a specific order determined by the coding sequence in a polynucleotide encoding the polypeptide.

A DNA construct is an assembly of DNA molecules linked together that provide one or more expression cassettes. The DNA construct may be a plasmid that is enabled for self-replication in a bacterial cell and contains various endonuclease enzyme restriction sites that are useful for introducing DNA molecules that provide functional genetic elements, i.e., promoters, introns, leaders, coding sequences, 3′ termination regions, among others; or a DNA construct may be a linear assembly of DNA molecules, such as an expression cassette. The expression cassette contained within a DNA construct comprises the necessary genetic elements to provide transcription of a messenger RNA. The expression cassette can be designed to express in prokaryote cells or eukaryotic cells. Expression cassettes of the embodiments of the present disclosure are designed to express in plant cells.

The DNA molecules of embodiments of the disclosure are provided in expression cassettes for expression in an organism of interest. The cassette will include 5′ and 3′ regulatory sequences operably linked to a coding sequence. “Operably linked” means that the nucleic acid sequences being linked are contiguous and, where necessary to join two protein coding regions, contiguous and in the same reading frame. Operably linked is intended to indicate a functional linkage between a promoter and a second sequence, wherein the promoter sequence initiates and mediates transcription of the DNA sequence corresponding to the second sequence. The cassette may additionally contain at least one additional gene to be co-transformed into the organism. Alternatively, the additional gene(s) can be provided on multiple expression cassettes or multiple DNA constructs.

The expression cassette will include in the 5′ to 3′ direction of transcription: a transcriptional and translational initiation region, a coding region, and a transcriptional and translational termination region functional in the organism serving as a host. The transcriptional initiation region (i.e., the promoter) may be native or analogous, or foreign or heterologous to the host organism. Additionally, the promoter may be the natural sequence or alternatively a synthetic sequence. The expression cassettes may additionally contain 5′ leader sequences in the expression cassette construct. Such leader sequences can act to enhance translation.

It is to be understood that as used herein the term “transgenic” includes any cell, cell line, callus, tissue, plant part, or plant, the genotype of which has been altered by the presence of a heterologous nucleic acid including those transgenics initially so altered as well as those created by sexual crosses or asexual propagation from the initial transgenic. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

A transgenic “event” is produced by transformation of plant cells with a heterologous DNA construct(s), including a nucleic acid expression cassette that comprises a transgene of interest, the regeneration of a population of plants resulting from the insertion of the transgene into the genome of the plant, and selection of a particular plant characterized by insertion into a particular genome location. An event is characterized phenotypically by the expression of the transgene. At the genetic level, an event is part of the genetic makeup of a plant. The term “event” also refers to progeny produced by a sexual outcross between the transformant and another variety that include the heterologous DNA. Even after repeated back-crossing to a recurrent parent, the inserted DNA and flanking DNA from the transformed parent is present in the progeny of the cross at the same chromosomal location. The term “event” also refers to DNA from the original transformant comprising the inserted DNA and flanking sequence immediately adjacent to the inserted DNA that would be expected to be transferred to a progeny that receives inserted DNA including the transgene of interest as the result of a sexual cross of one parental line that includes the inserted DNA (e.g., the original transformant and progeny resulting from selfing) and a parental line that does not contain the inserted DNA.

An insect resistant DP-033121-3 corn plant can be bred by first sexually crossing a first parental corn plant consisting of a corn plant grown from the transgenic DP-033121-3 corn plant and progeny thereof derived from transformation with the expression cassettes of the embodiments of the present disclosure that confers insect resistance, and a second parental corn plant that lacks insect resistance, thereby producing a plurality of first progeny plants; and then selecting a first progeny plant that is resistant to insects; and selfing the first progeny plant, thereby producing a plurality of second progeny plants; and then selecting from the second progeny plants an insect resistant plant. These steps can further include the back-crossing of the first insect resistant progeny plant or the second insect resistant progeny plant to the second parental corn plant or a third parental corn plant, thereby producing a corn plant that is resistant to insects.

As used herein, the term “plant” includes reference to whole plants, plant organs (e.g., leaves, stems, roots, etc.), seeds, plant cells, and progeny of same. Parts of transgenic plants understood to be within the scope of the disclosure comprise, for example, plant cells, protoplasts, tissues, callus, embryos as well as flowers, stems, fruits, leaves, and roots originating in transgenic plants or their progeny previously transformed with a DNA molecule of the disclosure and therefore consisting at least in part of transgenic cells, are also an embodiment of the present disclosure.

As used herein, the term “plant cell” includes, without limitation, seeds, suspension cultures, embryos, meristematic regions, callus tissue, leaves, roots, shoots, gametophytes, sporophytes, pollen, and microspores. The class of plants that can be used in the methods of the disclosure is generally as broad as the class of higher plants amenable to transformation techniques, including both monocotyledonous and dicotyledonous plants.

“Transformation” refers to the transfer of a nucleic acid fragment into the genome of a host organism, resulting in genetically stable inheritance. Host organisms containing the transformed nucleic acid fragments are referred to as “transgenic” organisms. Examples of methods of plant transformation include Agrobacterium-mediated transformation (De Blaere et al. (1987) Meth. Enzymol. 143:277) and particle-accelerated or “gene gun” transformation technology (Klein et al. (1987) Nature (London) 327:70-73; U.S. Pat. No. 4,945,050, incorporated herein by reference). Additional transformation methods are disclosed below.

Thus, isolated polynucleotides of the disclosure can be incorporated into recombinant constructs, typically DNA constructs, which are capable of introduction into and replication in a host cell. Such a construct can be a vector that includes a replication system and sequences that are capable of transcription and translation of a polypeptide-encoding sequence in a given host cell. A number of vectors suitable for stable transfection of plant cells or for the establishment of transgenic plants have been described in, e.g., Pouwels et al., (1985; Supp. 1987) Cloning Vectors: A Laboratory Manual, Weissbach and Weissbach (1989) Methods for Plant Molecular Biology, (Academic Press, New York); and Flevin et al., (1990) Plant Molecular Biology Manual, (Kluwer Academic Publishers). Typically, plant expression vectors include, for example, one or more cloned plant genes under the transcriptional control of 5′ and 3′ regulatory sequences and a dominant selectable marker. Such plant expression vectors also can contain, without limitation: a promoter regulatory region (e.g., a regulatory region controlling inducible or constitutive, environmentally- or developmentally-regulated, or cell- or tissue-specific expression), a transcription initiation start site, a ribosome binding site, an RNA processing signal, a transcription termination site, and/or a polyadenylation signal.

It is also to be understood that two different transgenic plants can also be crossed to produce progeny that contain two independently segregating added, exogenous genes. Selfing of appropriate progeny can produce plants that are homozygous for both added, exogenous genes. Back-crossing to a parental plant and out-crossing with a non-transgenic plant are also contemplated, as is vegetative propagation. Descriptions of other breeding methods that are commonly used for different traits and crops can be found in one of several references, e.g., Fehr, in Breeding Methods for Cultivar Development, Wilcos J. ed., American Society of Agronomy, Madison Wis. (1987).

### Seed Treatments

In one embodiment, seeds comprising event DP-033121-3 may be combined with a seed treatment formulation or compound.

The formula can be applied by such methods as drenching the growing medium including the seed with a solution or dispersion, mixing with growing medium and planting the seed in the treated growing medium, or various forms of seed treatments whereby the formulation is applied to the seed before it is planted.

In these methods the seed treatment will generally be used as a formulation or compound with an agriculturally suitable carrier comprising at least one of a liquid diluent, a solid diluent or a surfactant. A wide variety of formulations are suitable for this disclosure, the most suitable types of formulations depend upon the method of application.

Depending on the method of application, useful formulations include, without limitation: liquids such as solutions (including emulsifiable concentrates), suspensions, emulsions (including microemulsions and/or suspoemulsions) and the like which optionally can be thickened into gels.

Useful formulations further include, but are not limited to: solids such as dusts, powders, granules, pellets, tablets, films, and the like which can be water-dispersible (“wettable”) or water-soluble. Active ingredient can be (micro)encapsulated and further formed into a suspension or solid formulation; alternatively the entire formulation of active ingredient can be encapsulated (or “overcoated”). Encapsulation can control or delay release of the active ingredient. Sprayable formulations can be extended in suitable media and used at spray volumes from about one to several hundred liters per hectare.

The disclosure includes a seed contacted with a composition comprising a biologically effective amount of a seed treatment compound and an effective amount of at least one other biologically active compound or agent. The compositions used for treating seeds (or plant grown therefrom) according to this disclosure can also comprise an effective amount of one or more other biologically active compounds or agents. Suitable additional compounds or agents include, but are not limited to: insecticides, fungicides, nematocides, bactericides, acaricides, growth regulators such as rooting stimulants, chemosterilants, semiochemicals, repellents, attractants, pheromones, feeding stimulants, other biologically active compounds or entomopathogenic, viruses, bacteria or fungi to form a multi-component pesticide giving an even broader spectrum of agricultural utility. Examples of such biologically active compounds or agents with which compounds of this disclosure can be formulated are: insecticides such as abamectin, acephate, acetamiprid, amidoflumet (S-1955), avermectin, azadirachtin, azinphos-methyl, bifenthrin, binfenazate, buprofezin, carbofuran, chlorfenapyr, chlorfluazuron, chlorpyrifos, chlorpyrifos-methyl, chromafenozide, clothianidin, cyfluthrin, beta-cyfluthrin, cyhalothrin, lambda-cyhalothrin, cypermethrin, cyromazine, deltamethrin, diafenthiuron, diazinon, diflubenzuron, dimethoate, diofenolan, emamectin, endosulfan, esfenvalerate, ethiprole, fenothicarb, fenoxycarb, fenpropathrin, fenproximate, fenvalerate, fipronil, flonicamid, flucythrinate, tau-fluvalinate, flufenerim (UR-50701), flufenoxuron, fonophos, halofenozide, hexaflumuron, imidacloprid, indoxacarb, isofenphos, lufenuron, malathion, metaldehyde, methamidophos, methidathion, methomyl, methoprene, methoxychlor, monocrotophos, methoxyfenozide, nithiazin, novaluron, noviflumuron (XDE-007), oxamyl, parathion, parathion-methyl, permethrin, phorate, phosalone, phosmet, phosphamidon, pirimicarb, profenofos, pymetrozine, pyridalyl, pyriproxyfen, rotenone, spinosad, spiromesifin (BSN 2060), sulprofos, tebufenozide, teflubenzuron, tefluthrin, terbufos, tetrachlorvinphos, thiacloprid, thiamethoxam, thiodicarb, thiosultap-sodium, tralomethrin, trichlorfon and triflumuron; fungicides such as acibenzolar, azoxystrobin, benomyl, blasticidin-S, Bordeaux mixture (tribasic copper sulfate), bromuconazole, carpropamid, captafol, captan, carbendazim, chloroneb, chlorothalonil, copper oxychloride, copper salts, cyflufenamid, cymoxanil, cyproconazole, cyprodinil, (S)-3,5-dichloro-N-(3-chloro-1-ethyl-1-methyl-2-oxopropyl)-4-methylbenzamide (RH 7281), diclocymet (S-2900), diclomezine, dicloran, difenoconazole, (S)-3,5-d ihydro-5-methyl-2-(methylthio)-5-phenyl-3-(phenyl-amino)-4H-imidazol-4-one (RP 407213), dimethomorph, dimoxystrobin, diniconazole, diniconazole-M, dodine, edifenphos, epoxiconazole, famoxadone, fenamidone, fenarimol, fenbuconazole, fencaramid (SZX0722), fenpiclonil, fenpropidin, fenpropimorph, fentin acetate, fentin hydroxide, fluazinam, fludioxonil, flumetover (RPA 403397), flumorf/flumorlin (SYP-L190), fluoxastrobin (HEC 5725), fluquinconazole, flusilazole, flutolanil, flutriafol, folpet, fosetyl-aluminum, furalaxyl, furametapyr (S-82658), hexaconazole, ipconazole, iprobenfos, iprodione, isoprothiolane, kasugamycin, kresoxim-methyl, mancozeb, maneb, mefenoxam, mepronil, metalaxyl, metconazole, metominostrobin/fenominostrobin (SSF-126), metrafenone (AC 375839), myclobutanil, neo-asozin (ferric methanearsonate), nicobifen (BAS 510), orysastrobin, oxadixyl, penconazole, pencycuron, probenazole, prochloraz, propamocarb, propiconazole, proquinazid (DPX-KQ926), prothioconazole (JAU 6476), pyrifenox, pyraclostrobin, pyrimethanil, pyroquilon, quinoxyfen, spiroxamine, sulfur, tebuconazole, tetraconazole, thiabendazole, thifluzamide, thiophanate-methyl, thiram, tiadinil, triadimefon, triadimenol, tricyclazole, trifloxystrobin, triticonazole, validamycin and vinclozolin; nematocides such as aldicarb, oxamyl and fenamiphos; bactericides such as streptomycin; and acaricides such as amitraz, chinomethionat, chlorobenzilate, cyhexatin, dicofol, dienochlor, etoxazole, fenazaquin, fenbutatin oxide, fenpropathrin, fenpyroximate, hexythiazox, propargite, pyridaben and tebufenpyrad.

Examples of entomopathic viruses include, but are not limited to, species classified as baculoviruses, ascoviruses, iridoviruses, parvoviruses, polydnavirusespoxviruses, reoviruses and tetraviruses. Examples also include entomopathoic viruses that have been genetically modified with additional beneficial properties (Gramkow, A. W. et al., 2010 Virology Journal 7, art. no. 143; Shim, et al., 2009 Journal of Asia-pacific Entomology 12(4): 217-220).

Examples of entomopathic bacteria include, but are not limited to, species within the genera Bacillus (including B. cereus, B. popilliae, B. sphaericus and B. thuringiensis), Enterococcus, Fischerella, Lysinibacillus, Photorhabdus, Pseudomonas, Saccharopolyspora, Streptomyces, Xenorhabdus and Yersinia (see, for example, Barry, C., 2012 Journal of Invertebrate Pathology 109(1): 1-10; Sanchis, V., 2011 Agronomy for Sustainable Development 31(1): 217-231; Mason, K. L., et al., 2011 mBio 2(3): e00065-11; Muratoglu, H., et al., 2011 Turkish Journal of Biology 35(3): 275-282; Hincliffe, S. J., et al., 2010 The Open Toxinology Journal 3: 101-118; Kirst, H. A., 2010 Journal of Antibiotics 63(3): 101-111; Shu, C. and Zhang, J., 2009 Recent Patents on DNA and Gene Sequences 3(1): 26-28; Becher, P. J., et al., 2007 Phytochemistry 68(19): 2493-2497; Dodd, S. J., et al., 2006 Applied and Environmental Microbiology 72(10): 6584-6592; Zhang, J., et al. 1997 Journal of Bacteriology 179(13): 4336-4341.

Examples of entomopathic fungi include, but are not limited to species within the genera Beauveria (e.g., B. bassiana), Cordyceps, Lecanicillium, Metarhizium (e.g., M. anisopliae), Nomuraea and Paecilomyces (US20120128648, WO2011099022, US20110038839, U.S. Pat. No. 7,416,880, U.S. Pat. No. 6,660,290; Tang, L.-C. and Hou, R. F., 1998 Entomolgia Experimentalis et Applicata 88(1): 25-30) Examples of entomopathic nematodes include, but are not limited to, species within the genera Heterorhabditis and Steinernema (U.S. Pat. No. 6,184,434).

A general reference for these agricultural protectants is The Pesticide Manual, 12th Edition, C. D. S. Tomlin, Ed., British Crop Protection Council, Farnham, Surrey, U. K., 2000, L. G. Copping, ed., 2009 The Manual of Biocontrol Agents: A World Compendium (4^{th} ed., CABI Publishing); and Dev, S. and Koul, O., 1997 Insecticides of Natural Origin, CRC Press; EPA Biopesticides web publication, last viewed on May 25, 2012).

### Insect Resistance Management and Event Stacking

In one embodiment, the efficacy of event DP-033121-3 against target pests is increased and the development of resistant insects is reduced by use of a non-transgenic “refuge”—a section of non-insecticidal corn or other crop.

The United States Environmental Protection Agency publishes the requirements for use with transgenic crops producing a single Bt protein active against target pests, see: (epa.gov/oppbppdl/biopesticides/pips/bt_corn_refuge_{—}2006. htm, which can be accessed using the www prefix). In addition, the National Corn Growers Association, on their website: (ncga.com/insect-resistance-management-fact-sheet-bt-corn, which can be accessed using the www prefix) also provides similar guidance regarding refuge requirements.

Expression in a plant of two or more insecticidal compositions toxic to the same insect species, each insecticide being expressed at levels high enough to effectively delay the onset of resistance, would be another way to achieve control of the development of resistance. Roush et al., (The Royal Society. Phil. Trans. R. Soc. Lond. B. (1998) 353, 1777-1786) for example, outlines two-toxin strategies, also called “pyramiding” or “stacking,” for management of insecticidal transgenic crops. Stacking or pyramiding of two different proteins each effective against the target pests and with little or no cross-resistance can allow for use of a smaller refuge. The U.S. Environmental Protection Agency requires significantly less (generally 5%) structured refuge of non-Bt corn be planted than for single trait products (generally 20%). There are various ways of providing the IRM effects of a refuge, including various geometric planting patterns in the fields and in-bag seed mixtures, as discussed further by Roush et al. (The Royal Society. Phil. Trans. R. Soc. Lond. B. (1998) 353, 1777-1786)

In certain embodiments the event of the present disclosure can be “stacked”, or combined, with any combination of polynucleotide sequences of interest in order to create plants with a desired trait. A trait, as used herein, refers to the phenotype derived from a particular sequence or groups of sequences. For example, the event of the present disclosure may be stacked with any other polynucleotides encoding polypeptides of interest.

In one embodiment, maize event DP-033121-3 can be stacked with other genes conferring pesticidal and/or insecticidal activity, such as other Bacillus thuringiensis toxic proteins (described in U.S. Pat. Nos. 5,366,892; 5,747,450; 5,737,514; 5,723,756; 5,593,881; and Geiser et al. (1986) Gene 48:109), lectins (Van Damme et al. (1994) Plant Mol. Biol. 24:825, pentin (described in U.S. Pat. No. 5,981,722), and the like.

The combinations generated can also include multiple copies of any one of the polynucleotides of interest. The polynucleotides of the present disclosure can also be stacked with any other gene or combination of genes to produce plants with a variety of desired trait combinations including, but not limited to, balanced amino acids (e.g., hordothionins (U.S. Pat. Nos. 5,990,389; 5,885,801; 5,885,802; and 5,703,409); barley high lysine (Williamson et al. (1987) Eur. J. Biochem. 165:99-106; and WO 98/20122) and high methionine proteins (Pedersen et al. (1986) J. Biol. Chem. 261:6279; Kirihara et al. (1988) Gene 71:359 and Musumura et al. (1989) Plant Mol. Biol. 12:123); and thioredoxins (Sewalt et al., U.S. Pat. No. 7,009,087).

The polynucleotides of the present disclosure can also be stacked with traits desirable for disease or herbicide resistance (e.g., fumonisin detoxification genes (U.S. Pat. No. 5,792,931); avirulence and disease resistance genes (Jones et al. (1994) Science 266:789; Martin et al. (1993) Science 262:1432; Mindrinos et al. (1994) Cell 78:1089); acetolactate synthase (ALS) mutants that lead to herbicide resistance such as the S4 and/or Hra mutations; inhibitors of glutamine synthase such as phosphinothricin or basta (e.g., bar gene); and glyphosate resistance (EPSPS gene)); and traits desirable for processing or process products such as high oil (e.g., U.S. Pat. No. 6,232,529); modified oils (e.g., fatty acid desaturase genes (U.S. Pat. No. 5,952,544; WO 94/11516)); modified starches (e.g., ADPG pyrophosphorylases (AGPase), starch synthases (SS), starch branching enzymes (SBE), and starch debranching enzymes (SDBE)); and polymers or bioplastics (e.g., U.S. Pat. No. 5,602,321; beta-ketothiolase, polyhydroxybutyrate synthase, and acetoacetyl-CoA reductase (Schubert et al. (1988) J. Bacteriol. 170:5837-5847) facilitate expression of polyhydroxyalkanoates (PHAs)). One could also combine the polynucleotides of the present disclosure with polynucleotides providing agronomic traits such as male sterility (e.g., see U.S. Pat. No. 5,583,210), stalk strength, flowering time, or transformation technology traits such as cell cycle regulation or gene targeting (e.g., WO 99/61619, WO 00/17364, and WO 99/25821).

Non-limiting examples of events that may be combined with the event of the present disclosure are shown in Table 1.

TABLE 1

| Event | Company | Description |
|---|---|---|
| 176 | Syngenta Seeds, Inc. | Insect-resistant maize produced by inserting the |
| | | cry1Ab gene from Bacillus thuringiensis subsp. |
| | | kurstaki. The genetic modification affords |
| | | resistance to attack by the European corn borer |
| | | (ECB). |
| 3751IR | Pioneer Hi-Bred | Selection of somaclonal variants by culture of |
| | International Inc. | embryos on imidazolinone containing media. |
| 676, 678, 680 | Pioneer Hi-Bred | Male-sterile and glufosinate ammonium herbicide |
| | International Inc. | tolerant maize produced by inserting genes |
| | | encoding DNA adenine methylase and |
| | | phosphinothricin acetyltransferase (PAT) from |
| | | Escherichia coli and Streptomyces |
| | | viridochromogenes, respectively. |
| B16 (DLL25) | Dekalb Genetics | Glufosinate ammonium herbicide tolerant maize |
| | Corporation | produced by inserting the gene encoding |
| | | phosphinothricin acetyltransferase (PAT) from |
| | | Streptomyces hygroscopicus. |
| BT11 (X4334CBR, | Syngenta Seeds, Inc. | Insect-resistant and herbicide tolerant maize |
| X4734CBR) | | produced by inserting the cry1Ab gene from |
| | | Bacillus thuringiensis subsp. kurstaki, and the |
| | | phosphinothricin N-acetyltransferase (PAT) |
| | | encoding gene from S. viridochromogenes. |
| BT11 × | Syngenta Seeds, Inc. | Stacked insect resistant and herbicide tolerant |
| GA21 | | maize produced by conventional cross breeding |
| | | of parental lines BT11 (OECD unique identifier: |
| | | SYN-BTO11-1) and GA21 (OECD unique |
| | | identifier: MON-OOO21-9). |
| BT11 × | Syngenta Seeds, Inc. | Stacked insect resistant and herbicide tolerant |
| MIR162 | | maize produced by conventional cross breeding |
| | | of parental lines BT11 (OECD unique identifier: |
| | | SYN-BT011-1) and MIR162 (OECD unique |
| | | identifier: SYN-IR162-4). Resistance to the |
| | | European Corn Borer and tolerance to the |
| | | herbicide glufosinate ammonium (Liberty) is |
| | | derived from BT11, which contains the cry1Ab |
| | | gene from Bacillus thuringiensis subsp. kurstaki, |
| | | and the phosphinothricin N-acetyltransferase |
| | | (PAT) encoding gene from S. |
| | | viridochromogenes. Resistance to other |
| | | lepidopteran pests, including H. zea, S. |
| | | frugiperda, A. ipsilon, and S. albicosta, is derived |
| | | from MIR162, which contains the vip3Aa gene |
| | | from Bacillus thuringiensis strain AB88. |
| BT11 × | Syngenta Seeds, Inc. | Bacillus thuringiensis Cry1Ab delta-endotoxin |
| MIR162 × | | protein and the genetic material necessary for its |
| MIR604 | | production (via elements of vector pZO1502) in |
| | | Event Bt11 corn (OECD Unique Identifier: SYN- |
| | | BTO11-1) × Bacillus thuringiensis Vip3Aa20 |
| | | insecticidal protein and the genetic material |
| | | necessary for its production (via elements of |
| | | vector pNOV1300) in Event MIR162 maize |
| | | (OECD Unique Identifier: SYN-IR162-4) × |
| | | modified Cry3A protein and the genetic material |
| | | necessary for its production (via elements of |
| | | vector pZM26) in Event MIR604 corn (OECD |
| | | Unique Identifier: SYN-IR6O4-5). |
| BT11 × | Syngenta Seeds, Inc. | Resistance to coleopteran pests, particularly |
| MIR162 × | | corn rootworm pests (Diabrotica spp.) and |
| MIR604 × | | several lepidopteran pests of corn, including |
| GA21 | | European corn borer (ECB, Ostrinia nubilalis), |
| | | corn earworm (CEW, Helicoverpa zea), fall army |
| | | worm (FAW, Spodoptera frugiperda), and black |
| | | cutworm (BCW, Agrotis ipsilon); tolerance to |
| | | glyphosate and glufosinate-ammonium |
| | | containing herbicides. |
| BT11 × | Syngenta Seeds, Inc. | Stacked insect resistant and herbicide tolerant |
| MIR604 | | maize produced by conventional cross breeding |
| | | of parental lines BT11 (OECD unique identifier: |
| | | SYN-BT011-1) and MIR604 (OECD unique |
| | | identifier: SYN-IR6O5-5). Resistance to the |
| | | European Corn Borer and tolerance to the |
| | | herbicide glufosinate ammonium (Liberty) is |
| | | derived from BT11, which contains the cry1Ab |
| | | gene from Bacillus thuringiensis subsp. kurstaki, |
| | | and the phosphinothricin N-acetyltransferase |
| | | (PAT) encoding gene from S. |
| | | viridochromogenes. Corn rootworm-resistance is |
| | | derived from MIR604 which contains the mcry3A |
| | | gene from Bacillus thuringiensis. |
| BT11 × | Syngenta Seeds, Inc. | Stacked insect resistant and herbicide tolerant |
| MIR604 × | | maize produced by conventional cross breeding |
| GA21 | | of parental lines BT11 (OECD unique identifier: |
| | | SYN-BTO11-1), MIR604 (OECD unique |
| | | identifier: SYN-IR6O5-5) and GA21 (OECD |
| | | unique identifier: MON-OOO21-9). Resistance to |
| | | the European Corn Borer and tolerance to the |
| | | herbicide glufosinate ammonium (Liberty) is |
| | | derived from BT11, which contains the cry1Ab |
| | | gene from Bacillus thuringiensis subsp. kurstaki, |
| | | and the phosphinothricin N-acetyltransferase |
| | | (PAT) encoding gene from S. |
| | | viridochromogenes. Corn rootworm-resistance is |
| | | derived from MIR604 which contains the mcry3A |
| | | gene from Bacillus thuringiensis. Tolerance to |
| | | glyphosate herbicide is derived from GA21 which |
| | | contains a a modified EPSPS gene from maize. |
| CBH-351 | Aventis CropScience | Insect-resistant and glufosinate ammonium |
| | | herbicide tolerant maize developed by inserting |
| | | genes encoding Cry9C protein from Bacillus |
| | | thuringiensis subsp tolworthi and |
| | | phosphinothricin acetyltransferase (PAT) from |
| | | Streptomyces hygroscopicus. |
| DAS-06275-8 | DOW AgroSciences | Lepidopteran insect resistant and glufosinate |
| | LLC | ammonium herbicide-tolerant maize variety |
| | | produced by inserting the cry1F gene from |
| | | Bacillus thuringiensis var aizawai and the |
| | | phosphinothricin acetyltransferase (PAT) from |
| | | Streptomyces hygroscopicus. |
| DAS-59122-7 | DOW AgroSciences | Corn rootworm-resistant maize produced by |
| | LLC and Pioneer Hi- | inserting the cry34Ab1 and cry35Ab1 genes from |
| | Bred International Inc. | Bacillus thuringiensis strain PS149B1. The PAT |
| | | encoding gene from Streptomyces |
| | | viridochromogenes was introduced as a |
| | | selectable marker. |
| DAS-59122-7 × | DOW AgroSciences | Stacked insect resistant and herbicide tolerant |
| NK603 | LLC and Pioneer Hi- | maize produced by conventional cross breeding |
| | Bred International Inc. | of parental lines DAS-59122-7 (OECD unique |
| | | identifier: DAS-59122-7) with NK603 (OECD |
| | | unique identifier: MON-OO6O3-6). Corn |
| | | rootworm-resistance is derived from DAS-59122- |
| | | 7 which contains the cry34Ab1 and cry35Ab1 |
| | | genes from Bacillus thuringiensis strain |
| | | PS149B1. Tolerance to glyphosate herbicide is |
| | | derived from NK603. |
| TC1507 × | DOW AgroSciences | Stacked insect resistant and herbicide tolerant |
| NK603 | LLC | corn hybrid derived from conventional cross- |
| | | breeding of the parental lines 1507 (OECD |
| | | identifier: DAS-O15O7-1) and NK603 (OECD |
| | | identifier: MON-OO6O3-6). |
| DBT418 | Dekalb Genetics | Insect-resistant and glufosinate ammonium |
| | Corporation | herbicide tolerant maize developed by inserting |
| | | genes encoding Cry1AC protein from Bacillus |
| | | thuringiensis subsp kurstaki and phosphinothricin |
| | | acetyltransferase (PAT) from Streptomyces |
| | | hygroscopicus |
| DAS-59122-7 × | DOW AgroSciences | Stacked insect resistant and herbicide tolerant |
| TC1507 × | LLC and Pioneer Hi- | maize produced by conventional cross breeding |
| NK603 | Bred International Inc. | of parental lines DAS-59122-7 (OECD unique |
| | | identifier: DAS-59122-7) and TC1507 (OECD |
| | | unique identifier: DAS-O15O7-1) with NK603 |
| | | (OECD unique identifier: MON-OO6O3-6). Corn |
| | | rootworm-resistance is derived from DAS-59122- |
| | | 7 which contains the cry34Ab1 and cry35Ab1 |
| | | genes from Bacillus thuringiensis strain |
| | | PS149B1. Lepidopteran resistance and tolerance |
| | | to glufosinate ammonium herbicide is derived |
| | | from TC1507. Tolerance to glyphosate herbicide |
| | | is derived from NK603. |
| DK404SR | BASF Inc. | Somaclonal variants with a modified acetyl-CoA- |
| | | carboxylase (ACCase) were selected by culture |
| | | of embryos on sethoxydim enriched medium. |
| Event | Syngenta Seeds, Inc. | Maize line expressing a heat stable alpha- |
| 3272 | | amylase gene amy797E for use in the dry-grind |
| | | ethanol process. The phosphomannose |
| | | isomerase gene from E. coli was used as a |
| | | selectable marker. |
| Event | Pioneer Hi-Bred | Maize event expressing tolerance to glyphosate |
| 98140 | International Inc. | herbicide, via expression of a modified bacterial |
| | | glyphosate N-acetlytransferase, and ALS- |
| | | inhibiting herbicides, vial expression of a |
| | | modified form of the maize acetolactate synthase |
| | | enzyme. |
| EXP1910IT | Syngenta Seeds, Inc. | Tolerance to the imidazolinone herbicide, |
| | (formerly Zeneca | imazethapyr, induced by chemical mutagenesis |
| | Seeds) | of the acetolactate synthase (ALS) enzyme using |
| | | ethyl methanesulfonate (EMS). |
| GA21 | Syngenta Seeds, Inc. | Introduction, by particle bombardment, of a |
| | (formerly Zeneca | modified 5-enolpyruvyl shikimate-3-phosphate |
| | Seeds) | synthase (EPSPS), an enzyme involved in the |
| | | shikimate biochemical pathway for the |
| | | production of the aromatic amino acids. |
| GA21 × | Monsanto Company | Stacked insect resistant and herbicide tolerant |
| MON810 | | corn hybrid derived from conventional cross- |
| | | breeding of the parental lines GA21 (OECD |
| | | identifier: MON-OOO21-9) and MON810 (OECD |
| | | identifier: MON-OO81O-6). |
| IT | Pioneer Hi-Bred | Tolerance to the imidazolinone herbicide, |
| | International Inc. | imazethapyr, was obtained by in vitro selection of |
| | | somaclonal variants. |
| LY038 | Monsanto Company | Altered amino acid composition, specifically |
| | | elevated levels of lysine, through the introduction |
| | | of the cordapA gene, derived from |
| | | Corynebacterium glutamicum, encoding the |
| | | enzyme dihydrodipicolinate synthase (cDHDPS). |
| MIR162 | Syngenta Seeds, Inc. | Insect-resistant maize event expressing a Vip3A |
| | | protein from Bacillus thuringiensis and the |
| | | Escherichia coli PMI selectable marker |
| MIR604 | Syngenta Seeds, Inc. | Corn rootworm resistant maize produced by |
| | | transformation with a modified cry3A gene. The |
| | | phosphomannose isomerase gene from E. coli |
| | | was used as a selectable marker. |
| MIR604 × | Syngenta Seeds, Inc. | Stacked insect resistant and herbicide tolerant |
| GA21 | | maize produced by conventional cross breeding |
| | | of parental lines MIR604 (OECD unique |
| | | identifier: SYN-IR6O5-5) and GA21 (OECD |
| | | unique identifier: MON-OOO21-9). Corn |
| | | rootworm-resistance is derived from MIR604 |
| | | which contains the mcry3A gene from Bacillus |
| | | thuringiensis. Tolerance to glyphosate herbicide |
| | | is derived from GA21. |
| MON802 | Monsanto Company | Insect-resistant and glyphosate herbicide tolerant |
| | | maize produced by inserting the genes encoding |
| | | the Cry1Ab protein from Bacillus thuringiensis |
| | | and the 5-enolpyruvylshikimate-3-phosphate |
| | | synthase (EPSPS) from A. tumefaciens strain |
| | | CP4. |
| MON809 | Pioneer Hi-Bred | Resistance to European corn borer (Ostrinia |
| | International Inc. | nubilalis) by introduction of a synthetic cry1Ab |
| | | gene. Glyphosate resistance via introduction of |
| | | the bacterial version of a plant enzyme, 5- |
| | | enolpyruvyl shikimate-3-phosphate synthase |
| | | (EPSPS). |
| MON810 | Monsanto Company | Insect-resistant maize produced by inserting a |
| | | truncated form of the crylAb gene from Bacillus |
| | | thuringiensis subsp. kurstaki HD-1. The genetic |
| | | modification affords resistance to attack by the |
| | | European corn borer (ECB). |
| MON810 × | Monsanto Company | Stacked insect resistant and enhanced lysine |
| LY038 | | content maize derived from conventional cross- |
| | | breeding of the parental lines MON810 (OECD |
| | | identifier: MON-OO81O-6) and LY038 (OECD |
| | | identifier: REN-OOO38-3). |
| MON810 × | Monsanto Company | Stacked insect resistant and glyphosate tolerant |
| MON88017 | | maize derived from conventional cross-breeding |
| | | of the parental lines MON810 (OECD identifier: |
| | | MON-OO81O-6) and MON88017 (OECD |
| | | identifier: MON-88O17-3). European corn borer |
| | | (ECB) resistance is derived from a truncated |
| | | form of the crylAb gene from Bacillus |
| | | thuringiensis subsp. kurstaki HD-1 present in |
| | | MON810. Corn rootworm resistance is derived |
| | | from the cry3Bb1 gene from Bacillus |
| | | thuringiensis subspecies kumamotoensis strain |
| | | EG4691 present in MON88017. Glyphosate |
| | | tolerance is derived from a 5- |
| | | enolpyruvylshikimate-3-phosphate synthase |
| | | (EPSPS) encoding gene from Agrobacterium |
| | | tumefaciens strain CP4 present in MON88017. |
| MON832 | Monsanto Company | Introduction, by particle bombardment, of |
| | | glyphosate oxidase (GOX) and a modified 5- |
| | | enolpyruvyl shikimate-3-phosphate synthase |
| | | (EPSPS), an enzyme involved in the shikimate |
| | | biochemical pathway for the production of the |
| | | aromatic amino acids. |
| MON863 | Monsanto Company | Corn root worm resistant maize produced by |
| | | inserting the cry3Bb1 gene from Bacillus |
| | | thuringiensis subsp. kumamotoensis. |
| MON863 × | Monsanto Company | Stacked insect resistant corn hybrid derived from |
| MON810 | | conventional cross-breeding of the parental lines |
| | | MON863 (OECD identifier: MON-OO863-5) and |
| | | MON810 (OECD identifier: MON-OO81O-6) |
| MON863 × | Monsanto Company | Stacked insect resistant and herbicide tolerant |
| MON810 × | | corn hybrid derived from conventional cross- |
| NK603 | | breeding of the stacked hybrid MON-OO863-5 × |
| | | MON-OO81O-6 and NK603 (OECD |
| | | identifier: MON-OO6O3-6). |
| MON863 × | Monsanto Company | Stacked insect resistant and herbicide tolerant |
| NK603 | | corn hybrid derived from conventional cross- |
| | | breeding of the parental lines MON863 (OECD |
| | | identifier: MON-OO863-5) and NK603 (OECD |
| | | identifier: MON-OO6O3-6). |
| MON87460 | Monsanto Company | MON 87460 was developed to provide reduced |
| | | yield loss underwater-limited conditions |
| | | compared to conventional maize. Efficacy in |
| | | MON 87460 is derived by expression of the |
| | | inserted Bacillus subtilis cold shock protein B |
| | | (CspB). |
| MON88017 | Monsanto Company | Corn rootworm-resistant maize produced by |
| | | inserting the cry3Bb1 gene from Bacillus |
| | | thuringiensis subspecies kumamotoensis strain |
| | | EG4691. Glyphosate tolerance derived by |
| | | inserting a 5-enolpyruvylshikimate-3-phosphate |
| | | synthase (EPSPS) encoding gene from |
| | | Agrobacterium tumefaciens strain CP4. |
| MON89034 | Monsanto Company | Maize event expressing two different insecticidal |
| | | proteins from Bacillus thuringiensis providing |
| | | resistance to number of lepidopteran pests. |
| MON89034 × | Monsanto Company | Stacked insect resistant and glyphosate tolerant |
| MON88017 | | maize derived from conventional cross-breeding |
| | | of the parental lines MON89034 (OECD |
| | | identifier: MON-89O34-3) and MON88017 |
| | | (OECD identifier: MON-88O17-3). Resistance to |
| | | Lepidopteran insects is derived from two cry |
| | | genes present in MON89043. Corn rootworm |
| | | resistance is derived from a single cry genes and |
| | | glyphosate tolerance is derived from the 5- |
| | | enolpyruvylshikimate-3-phosphate synthase |
| | | (EPSPS) encoding gene from Agrobacterium |
| | | tumefaciens present in MON88017. |
| MON89034 × | Monsanto Company | Stacked insect resistant and herbicide tolerant |
| NK603 | | maize produced by conventional cross breeding |
| | | of parental lines MON89034 (OECD identifier: |
| | | MON-89O34-3) with NK603 (OECD unique |
| | | identifier: MON-OO6O3-6). Resistance to |
| | | Lepidopteran insects is derived from two cry |
| | | genes present in MON89043. Tolerance to |
| | | glyphosate herbicide is derived from NK603. |
| MON89034 × | Monsanto Company | Stacked insect resistant and herbicide tolerant |
| TC1507 × | and Mycogen Seeds c/o | maize produced by conventional cross breeding |
| MON88017 × | Dow AgroSciences LLC | of parental lines: MON89034, TC1507, |
| DAS-59122-7 | | MON88017, and DAS-59122. Resistance to the |
| | | above-ground and below-ground insect pests |
| | | and tolerance to glyphosate and glufosinate- |
| | | ammonium containing herbicides. |
| MS3 | Bayer CropScience | Male sterility caused by expression of the |
| | (Aventis | barnase ribonuclease gene from Bacillus |
| | CropScience(AgrEvo)) | amyloliquefaciens; PPT resistance was via PPT- |
| | | acetyltransferase (PAT). |
| MS6 | Bayer CropScience | Male sterility caused by expression of the |
| | (Aventis | barnase ribonuclease gene from Bacillus |
| | CropScience(AgrEvo)) | amyloliquefaciens; PPT resistance was via PPT- |
| | | acetyltransferase (PAT). |
| NK603 | Monsanto Company | Introduction, by particle bombardment, of a |
| | | modified 5-enolpyruvyl shikimate-3-phosphate |
| | | synthase (EPSPS), an enzyme involved in the |
| | | shikimate biochemical pathway for the |
| | | production of the aromatic amino acids. |
| NK603 × | Monsanto Company | Stacked insect resistant and herbicide tolerant |
| MON810 | | corn hybrid derived from conventional cross- |
| | | breeding of the parental lines NK603 (OECD |
| | | identifier: MON-OO6O3-6) and MON810 (OECD |
| | | identifier: MON-OO81O-6). |
| NK603 × | Monsanto Company | Stacked glufosinate ammonium and glyphosate |
| T25 | | herbicide tolerant maize hybrid derived from |
| | | conventional cross-breeding of the parental lines |
| | | NK603 (OECD identifier: MON-OO6O3-6) and |
| | | T25 (OECD identifier: ACS-ZM003-2). |
| T14, T25 | Bayer CropScience | Glufosinate herbicide tolerant maize produced by |
| | (Aventis | inserting the phosphinothricin N- |
| | CropScience(AgrEvo)) | acetyltransferase (PAT) encoding gene from the |
| | | aerobic actinomycete Streptomyces |
| | | viridochromogenes. |
| T25 × | Bayer CropScience | Stacked insect resistant and herbicide tolerant |
| MON810 | (Aventis | corn hybrid derived from conventional cross- |
| | CropScience(AgrEvo)) | breeding of the parental lines T25 (OECD |
| | | identifier: ACS-ZMOO3-2) and MON810 (OECD |
| | | identifier: MON-OO81O-6). |
| TC1507 | Mycogen (c/o Dow | Insect-resistant and glufosinate ammonium |
| | AgroSciences); Pioneer | herbicide tolerant maize produced by inserting |
| | (c/o DuPont) | the cry1F gene from Bacillus thuringiensis var. |
| | | aizawai and the phosphinothricin N- |
| | | acetyltransferase encoding gene from |
| | | Streptomyces viridochromogenes. |
| TC1507 × | DOW AgroSciences | Stacked insect resistant and herbicide tolerant |
| DAS-59122-7 | LLC and Pioneer Hi- | maize produced by conventional cross breeding |
| | Bred International Inc. | of parental lines TC1507 (OECD unique |
| | | identifier: DAS-O15O7-1) with DAS-59122-7 |
| | | (OECD unique identifier: DAS-59122-7). |
| | | Resistance to lepidopteran insects is derived |
| | | from TC1507 due the presence of the cry1F |
| | | gene from Bacillus thuringiensis var. aizawai. |
| | | Corn rootworm-resistance is derived from DAS- |
| | | 59122-7 which contains the cry34Ab1 and |
| | | cry35Ab1 genes from Bacillus thuringiensis |
| | | strain PS149B1. Tolerance to glufosinate |
| | | ammonium herbicide is derived from TC1507 |
| | | from the phosphinothricin N-acetyltransferase |
| | | encoding gene from Streptomyces |
| | | viridochromogenes. |

Other events with regulatory approval are well known to one skilled in the art and can be found at the Center for Environmental Risk Assessment (cera-gmc.org/?action=gm_crop_database, which can be accessed using the www prefix) and at the International Service for the Acquisition of Agri-Biotech Applications (isaaa.org/gmapprovaldatabase/default.asp, which can be accessed using the www prefix).

These stacked combinations can be created by any method including, but not limited to, cross-breeding plants by any conventional or TopCross® methodology, or genetic modification. If the sequences are stacked by genetically transforming the plants, the polynucleotide sequences of interest can be combined at any time and in any order. For example, a transgenic plant comprising one or more desired traits can be used as the target to introduce further traits by subsequent transformation. The traits can be introduced simultaneously in a co-transformation protocol with the polynucleotides of interest provided by any combination of transformation cassettes. Expression of the sequences can be driven by the same promoter or by different promoters. In certain cases, it may be desirable to introduce a transformation cassette that will suppress the expression of another polynucleotide of interest. This may be combined with any combination of other suppression cassettes or over-expression cassettes to generate the desired combination of traits in the plant. It is further recognized that polynucleotide sequences can be stacked at a desired genomic location using a site-specific recombination system. See, for example, WO99/25821, WO99/25854, WO99/25840, WO99/25855, and WO99/25853.

In another embodiment, the event of the disclosure can be combined with traits native to certain maize lines that can be identified by a quantitative trait locus (QTL).

The term “quantitative trait locus” or “QTL” refers to a polymorphic genetic locus with at least one allele that correlates with the differential expression of a phenotypic trait in at least one genetic background, e.g., in at least one breeding population or progeny. A QTL can act through a single gene mechanism or by a polygenic mechanism. Examples of QTL traits that may be combined with the event of the disclosure include, but are not limited to: Fusarium resistance (US Pat Pub No: 2010/0269212), Head Smut resistance (US Pat Pub No: 2010/0050291); Colleotrichum resistance (U.S. Pat. No. 8,062,847); and increased oil (U.S. Pat. No. 8,084,208).

In another embodiment, the event of the disclosure can be combined with genes that create a site for site specific DNA integration. This includes the introduction of FRT sites that may be used in the FLP/FRT system and/or Lox sites that may be used in the Cre/Lox system. For example, see Lyznik, et al., Site-Specific Recombination for Genetic Engineering in Plants, Plant Cell Rep (2003) 21:925-932 and WO 99/25821.

A “probe” is an isolated nucleic acid to which is attached a conventional detectable label or reporter molecule, e.g., a radioactive isotope, ligand, chemiluminescent agent, or enzyme. Such a probe is complementary to a strand of a target nucleic acid, in the case of the present disclosure, to a strand of isolated DNA from corn event DP-033121-3 whether from a corn plant or from a sample that includes DNA from the event. Probes according to the present disclosure include not only deoxyribonucleic or ribonucleic acids but also polyamides and other probe materials that bind specifically to a target DNA sequence and can be used to detect the presence of that target DNA sequence.

“Primers” are isolated nucleic acids that are annealed to a complementary target DNA strand by nucleic acid hybridization to form a hybrid between the primer and the target DNA strand, then extended along the target DNA strand by a polymerase, e.g., a DNA polymerase. Primer pairs of the disclosure refer to their use for amplification of a target nucleic acid sequence, e.g., by PCR or other conventional nucleic-acid amplification methods. “PCR” or “polymerase chain reaction” is a technique used for the amplification of specific DNA segments (see, U.S. Pat. Nos. 4,683,195 and 4,800,159; herein incorporated by reference).

Probes and primers are of sufficient nucleotide length to bind to the target DNA sequence specifically in the hybridization conditions or reaction conditions determined by the operator. This length may be of any length that is of sufficient length to be useful in a detection method of choice. Generally, 11 nucleotides or more in length, 18 nucleotides or more, and 22 nucleotides or more, are used. Such probes and primers hybridize specifically to a target sequence under high stringency hybridization conditions. Probes and primers according to embodiments of the present disclosure may have complete DNA sequence similarity of contiguous nucleotides with the target sequence, although probes differing from the target DNA sequence and that retain the ability to hybridize to target DNA sequences may be designed by conventional methods. Probes can be used as primers, but are generally designed to bind to the target DNA or RNA and are not used in an amplification process.

Specific primers can be used to amplify an integration fragment to produce an amplicon that can be used as a “specific probe” for identifying event DP-033121-3 in biological samples. When the probe is hybridized with the nucleic acids of a biological sample under conditions which allow for the binding of the probe to the sample, this binding can be detected and thus allow for an indication of the presence of event DP-033121-3 in the biological sample. Such identification of a bound probe has been described in the art. In an embodiment of the disclosure the specific probe is a sequence which, under optimized conditions, hybridizes specifically to a region within the 5′ or 3′ flanking region of the event and also comprises a part of the foreign DNA contiguous therewith. The specific probe may comprise a sequence of at least 80%, between 80 and 85%, between 85 and 90%, between 90 and 95%, and between 95 and 100% identical (or complementary) to a specific region of the event.

Methods for preparing and using probes and primers are described, for example, in Sambrook et al., Molecular Cloning: A Laboratory Manual, 2^{nd} ed., vol. 1-3, Cold Spring Harbor Laboratory Press, Cold Spring Harbor, N.Y. 1989 (hereinafter, “Sambrook et al., 1989”); Ausubel et al. eds., Current Protocols in Molecular Biology, Greene Publishing and Wiley-Interscience, New York, 1995 (with periodic updates) (hereinafter, “Ausubel et al., 1995”); and Innis et al., PCR Protocols: A Guide to Methods and Applications, Academic Press: San Diego, 1990. PCR primer pairs can be derived from a known sequence, for example, by using computer programs intended for that purpose such as the PCR primer analysis tool in Vector NTI version 6 (Informax Inc., Bethesda Md.); PrimerSelect (DNASTAR Inc., Madison, Wis.); and Primer (Version 0.5©, 1991, Whitehead Institute for Biomedical Research, Cambridge, Mass.). Additionally, the sequence can be visually scanned and primers manually identified using guidelines known to one of skill in the art.

A “kit” as used herein refers to a set of reagents for the purpose of performing the method embodiments of the disclosure, more particularly, the identification of event DP-033121-3 in biological samples. The kit of the disclosure can be used, and its components can be specifically adjusted, for purposes of quality control (e.g. purity of seed lots), detection of event DP-033121-3 in plant material, or material comprising or derived from plant material, such as but not limited to food or feed products. “Plant material” as used herein refers to material which is obtained or derived from a plant.

Primers and probes based on the flanking DNA and insert sequences disclosed herein can be used to confirm (and, if necessary, to correct) the disclosed sequences by conventional methods, e.g., by re-cloning and sequencing such sequences. The nucleic acid probes and primers of the present disclosure hybridize under stringent conditions to a target DNA sequence. Any conventional nucleic acid hybridization or amplification method can be used to identify the presence of DNA from a transgenic event in a sample. Nucleic acid molecules or fragments thereof are capable of specifically hybridizing to other nucleic acid molecules under certain circumstances. As used herein, two nucleic acid molecules are said to be capable of specifically hybridizing to one another if the two molecules are capable of forming an anti-parallel, double-stranded nucleic acid structure.

A nucleic acid molecule is said to be the “complement” of another nucleic acid molecule if they exhibit complete complementarity. As used herein, molecules are said to exhibit “complete complementarity” when every nucleotide of one of the molecules is complementary to a nucleotide of the other. Two molecules are said to be “minimally complementary” if they can hybridize to one another with sufficient stability to permit them to remain annealed to one another under at least conventional “low-stringency” conditions. Similarly, the molecules are said to be “complementary” if they can hybridize to one another with sufficient stability to permit them to remain annealed to one another under conventional “high-stringency” conditions. Conventional stringency conditions are described by Sambrook et al., 1989, and by Haymes et al., In: Nucleic Acid Hybridization, a Practical Approach, IRL Press, Washington, D.C. (1985). Departures from complete complementarity are therefore permissible, as long as such departures do not completely preclude the capacity of the molecules to form a double-stranded structure. In order for a nucleic acid molecule to serve as a primer or probe it need only be sufficiently complementary in sequence to be able to form a stable double-stranded structure under the particular solvent and salt concentrations employed.

In hybridization reactions, specificity is typically the function of post-hybridization washes, the critical factors being the ionic strength and temperature of the final wash solution. The thermal melting point (Tₘ) is the temperature (under defined ionic strength and pH) at which 50% of a complementary target sequence hybridizes to a perfectly matched probe. For DNA-DNA hybrids, the Tₘ can be approximated from the equation of Meinkoth and Wahl (1984) Anal. Biochem. 138:267-284: Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the Tₘ for the specific sequence and its complement at a defined ionic strength and pH. However, severely stringent conditions can utilize a hybridization and/or wash at 1, 2, 3, or 4° C. lower than the Tₘ; moderately stringent conditions can utilize a hybridization and/or wash at 6, 7, 8, 9, or 10° C. lower than the Tₘ; low stringency conditions can utilize a hybridization and/or wash at 11, 12, 13, 14, 15, or 20° C. lower than the Tₘ.

Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is preferred to increase the SSC concentration so that a higher temperature can be used. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, New York); and Ausubel et al., eds. (1995) and Sambrook et al. (1989).

As used herein, a substantially homologous sequence is a nucleic acid molecule that will specifically hybridize to the complement of the nucleic acid molecule to which it is being compared under high stringency conditions. Appropriate stringency conditions which promote DNA hybridization, for example, 6× sodium chloride/sodium citrate (SSC) at about 45° C., followed by a wash of 2×SSC at 50° C., are known to those skilled in the art or can be found in Ausubel et al. (1995), 6.3.1-6.3.6. Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of a destabilizing agent such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. A nucleic acid of the disclosure may specifically hybridize to one or more of the nucleic acid molecules unique to the DP-033121-3 event or complements thereof or fragments of either under moderately stringent conditions.

Methods of alignment of sequences for comparison are well known in the art. Thus, the determination of percent identity between any two sequences can be accomplished using a mathematical algorithm. Non-limiting examples of such mathematical algorithms are the algorithm of Myers and Miller (1988) CABIOS 4:11-17; the local homology algorithm of Smith et al. (1981) Adv. Appl. Math. 2:482; the homology alignment algorithm of Needleman and Wunsch (1970) J. Mol. Biol. 48:443-453; the search-for-similarity-method of Pearson and Lipman (1988) Proc. Natl. Acad. Sci. 85:2444-2448; the algorithm of Karlin and Altschul (1990) Proc. Natl. Acad. Sci. USA 87:2264, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0); the ALIGN PLUS program (version 3.0, copyright 1997); and GAP, BESTFIT, BLAST, FASTA, and TFASTA in the Wisconsin Genetics Software Package, Version 10 (available from Accelrys, 9685 Scranton Road, San Diego, Calif. 92121, USA). Alignments using these programs can be performed using the default parameters.

The CLUSTAL program is well described by Higgins and Sharp, Gene 73: 237-244 (1988); Higgins and Sharp, CABIOS 5: 151-153 (1989); Corpet, et al., Nucleic Acids Research 16: 10881-90 (1988); Huang, et al., Computer Applications in the Biosciences 8: 155-65 (1992), and Pearson, et al., Methods in Molecular Biology 24: 307-331 (1994). The ALIGN and the ALIGN PLUS programs are based on the algorithm of Myers and Miller (1988) supra. The BLAST programs of Altschul et al. (1990) J. Mol. Biol. 215:403 are based on the algorithm of Karlin and Altschul (1990) supra. The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Ausubel, et al., (1995). Alignment may also be performed manually by visual inspection.

To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used.

As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

Regarding the amplification of a target nucleic acid sequence (e.g., by PCR) using a particular amplification primer pair, “stringent conditions” are conditions that permit the primer pair to hybridize only to the target nucleic-acid sequence to which a primer having the corresponding wild-type sequence (or its complement) would bind and preferably to produce a unique amplification product, the amplicon, in a DNA thermal amplification reaction.

The term “specific for (a target sequence)” indicates that a probe or primer hybridizes under stringent hybridization conditions only to the target sequence in a sample comprising the target sequence.

As used herein, “amplified DNA” or “amplicon” refers to the product of nucleic acid amplification of a target nucleic acid sequence that is part of a nucleic acid template. For example, to determine whether a corn plant resulting from a sexual cross contains transgenic event genomic DNA from the corn plant of the disclosure, DNA extracted from the corn plant tissue sample may be subjected to a nucleic acid amplification method using a DNA primer pair that includes a first primer derived from flanking sequence adjacent to the insertion site of inserted heterologous DNA, and a second primer derived from the inserted heterologous DNA to produce an amplicon that is diagnostic for the presence of the event DNA. Alternatively, the second primer may be derived from the flanking sequence. The amplicon is of a length and has a sequence that is also diagnostic for the event. The amplicon may range in length from the combined length of the primer pairs plus one nucleotide base pair to any length of amplicon producible by a DNA amplification protocol. Alternatively, primer pairs can be derived from flanking sequence on both sides of the inserted DNA so as to produce an amplicon that includes the entire insert nucleotide sequence of the PHP36676 expression construct as well as the sequence flanking the transgenic insert. A member of a primer pair derived from the flanking sequence may be located a distance from the inserted DNA sequence, this distance can range from one nucleotide base pair up to the limits of the amplification reaction, or about 20,000 bp. The use of the term “amplicon” specifically excludes primer dimers that may be formed in the DNA thermal amplification reaction.

Nucleic acid amplification can be accomplished by any of the various nucleic acid amplification methods known in the art, including PCR. A variety of amplification methods are known in the art and are described, inter alia, in U.S. Pat. Nos. 4,683,195 and 4,683,202 and in Innis et al., (1990) supra. PCR amplification methods have been developed to amplify up to 22 Kb of genomic DNA and up to 42 Kb of bacteriophage DNA (Cheng et al., Proc. Natl. Acad. Sci. USA 91:5695-5699, 1994). These methods as well as other methods known in the art of DNA amplification may be used in the practice of the embodiments of the present disclosure. It is understood that a number of parameters in a specific PCR protocol may need to be adjusted to specific laboratory conditions and may be slightly modified and yet allow for the collection of similar results. These adjustments will be apparent to a person skilled in the art.

The amplicon produced by these methods may be detected by a plurality of techniques, including, but not limited to, Genetic Bit Analysis (Nikiforov, et al. Nucleic Acid Res. 22:4167-4175, 1994) where a DNA oligonucleotide is designed which overlaps both the adjacent flanking DNA sequence and the inserted DNA sequence. The oligonucleotide is immobilized in wells of a micro well plate. Following PCR of the region of interest (using one primer in the inserted sequence and one in the adjacent flanking sequence) a single-stranded PCR product can be hybridized to the immobilized oligonucleotide and serve as a template for a single base extension reaction using a DNA polymerase and labeled ddNTPs specific for the expected next base. Readout may be fluorescent or ELISA-based. A signal indicates presence of the insert/flanking sequence due to successful amplification, hybridization, and single base extension.

Another detection method is the pyrosequencing technique as described by Winge (2000) Innov. Pharma. Tech. 00:18-24. In this method an oligonucleotide is designed that overlaps the adjacent DNA and insert DNA junction. The oligonucleotide is hybridized to a single-stranded PCR product from the region of interest (one primer in the inserted sequence and one in the flanking sequence) and incubated in the presence of a DNA polymerase, ATP, sulfurylase, luciferase, apyrase, adenosine 5′ phosphosulfate and luciferin. dNTPs are added individually and the incorporation results in a light signal which is measured. A light signal indicates the presence of the transgene insert/flanking sequence due to successful amplification, hybridization, and single or multi-base extension.

Fluorescence polarization as described by Chen et al., (1999) Genome Res. 9:492-498 is also a method that can be used to detect an amplicon of the disclosure. Using this method an oligonucleotide is designed which overlaps the flanking and inserted DNA junction. The oligonucleotide is hybridized to a single-stranded PCR product from the region of interest (one primer in the inserted DNA and one in the flanking DNA sequence) and incubated in the presence of a DNA polymerase and a fluorescent-labeled ddNTP. Single base extension results in incorporation of the ddNTP. Incorporation can be measured as a change in polarization using a fluorometer. A change in polarization indicates the presence of the transgene insert/flanking sequence due to successful amplification, hybridization, and single base extension.

Taqman® (PE Applied Biosystems, Foster City, Calif.) is described as a method of detecting and quantifying the presence of a DNA sequence and is fully understood in the instructions provided by the manufacturer. Briefly, a FRET oligonucleotide probe is designed which overlaps the flanking and insert DNA junction. The FRET probe and PCR primers (one primer in the insert DNA sequence and one in the flanking genomic sequence) are cycled in the presence of a thermo stable polymerase and dNTPs. Hybridization of the FRET probe results in cleavage and release of the fluorescent moiety away from the quenching moiety on the FRET probe. A fluorescent signal indicates the presence of the flanking/transgene insert sequence due to successful amplification and hybridization.

Molecular beacons have been described for use in sequence detection as described in Tyangi et al. (1996) Nature Biotech. 14:303-308. Briefly, a FRET oligonucleotide probe is designed that overlaps the flanking and insert DNA junction. The unique structure of the FRET probe results in it containing secondary structure that keeps the fluorescent and quenching moieties in close proximity. The FRET probe and PCR primers (one primer in the insert DNA sequence and one in the flanking sequence) are cycled in the presence of a thermo stable polymerase and dNTPs. Following successful PCR amplification, hybridization of the FRET probe to the target sequence results in the removal of the probe secondary structure and spatial separation of the fluorescent and quenching moieties. A fluorescent signal results. A fluorescent signal indicates the presence of the flanking/transgene insert sequence due to successful amplification and hybridization.

A hybridization reaction using a probe specific to a sequence found within the amplicon is yet another method used to detect the amplicon produced by a PCR reaction.

Maize event DP-033121-3 is effective against insect pests including insects selected from the orders: Coleoptera, Diptera, Hymenoptera, Lepidoptera, Mallophaga, Homoptera, Hemiptera, Orthoptera, Thysanoptera, Dermaptera, Isoptera, Anoplura, Siphonaptera, Trichoptera, etc., particularly Coleoptera and Lepidoptera.

Insects of the order Lepidoptera include, but are not limited to, armyworms, cutworms, loopers, and heliothines in the family Noctuidae: Agrotis ipsilon Hufnagel (black cutworm); A. orthogonia Morrison (western cutworm); A. segetum Denis & Schiffermüller (turnip moth); A. subterranea Fabricius (granulate cutworm); Alabama argillacea Hübner (cotton leaf worm); Anticarsia gemmatalis Hübner (velvetbean caterpillar); Athetis mindara Barnes and McDunnough (rough skinned cutworm); Earias insulana Boisduval (spiny bollworm); E. vittella Fabricius (spotted bollworm); Egira (Xylomyges) curialis Grote (citrus cutworm); Euxoa messoria Harris (darksided cutworm); Helicoverpa armigera Hübner (American bollworm); H. zea Boddie (corn earworm or cotton bollworm); Heliothis virescens Fabricius (tobacco budworm); Hypena scabra Fabricius (green cloverworm); Hyponeuma taltula Schaus; (Mamestra configurata Walker (bertha armyworm); M. brassicae Linnaeus (cabbage moth); Melanchra picta Harris (zebra caterpillar); Mocis latipes Guenée (small mocis moth); Pseudaletia unipuncta Haworth (armyworm); Pseudoplusia includens Walker (soybean looper); Richia albicosta Smith (Western bean cutworm); Spodoptera frugiperda JE Smith (fall armyworm); S. exigua Hübner (beet armyworm); S. litura Fabricius (tobacco cutworm, cluster caterpillar); Trichoplusia ni Hübner (cabbage looper); borers, casebearers, webworms, coneworms, and skeletonizers from the families Pyralidae and Crambidae such as Achroia grisella Fabricius (lesser wax moth); Amyelois transitella Walker (naval orangeworm); Anagasta kuehniella Zeller (Mediterranean flour moth); Cadra cautella Walker (almond moth); Chilo partellus Swinhoe (spotted stalk borer); C. suppressalis Walker (striped stem/rice borer); C. terrenellus Pagenstecher (sugarcane stem borer); Corcyra cephalonica Stainton (rice moth); Crambus caliginosellus Clemens (corn root webworm); C. teterrellus Zincken (bluegrass webworm); Cnaphalocrocis medinalis Guenee (rice leaf roller); Desmia funeralis Hubner (grape leaffolder); Diaphania hyalinata Linnaeus (melon worm); D. nitidalis Stoll (pickleworm); Diatraea flavipennella Box; D. grandiosella Dyar (southwestern corn borer), D. saccharalis Fabricius (surgarcane borer); Elasmopalpus lignosellus Zeller (lesser cornstalk borer); Eoreuma loftini Dyar (Mexican rice borer); Ephestia elutella Hubner (tobacco (cacao) moth); Galleria mellonella Linnaeus (greater wax moth); Hedylepta accepta Butler (sugarcane leafroller); Herpetogramma licarsisalis Walker (sod webworm); Homoeosoma electellum Hulst (sunflower moth); Loxostege sticticalis Linnaeus (beet webworm); Maruca testulalis Geyer (bean pod borer); Orthaga thyrisalis Walker (tea tree web moth); Ostrinia nubilalis Hubner (European corn borer); Plodia interpunctella Hubner (Indian meal moth); Scirpophaga incertulas Walker (yellow stem borer); Udea rubigalis Guenee (celery leaftier); and leafrollers, budworms, seed worms, and fruit worms in the family Tortricidae Acleris gloverana Walsingham (Western blackheaded budworm); A. variana Fernald (Eastern blackheaded budworm); Adoxophyes orana Fischer von Rösslerstamm (summer fruit tortrix moth); Archips spp. including A. argyrospila Walker (fruit tree leaf roller) and A. rosana Linnaeus (European leaf roller); Argyrotaenia spp.; Bonagota salubricola Meyrick (Brazilian apple leafroller); Choristoneura spp.; Cochylis hospes Walsingham (banded sunflower moth); Cydia latiferreana Walsingham (filbertworm); C. pomonella Linnaeus (codling moth); Endopiza viteana Clemens (grape berry moth); Eupoecilia ambiguella Hubner (vine moth); Grapholita molesta Busck (oriental fruit moth); Lobesia botrana Denis & Schiffermüller (European grape vine moth); Platynota flavedana Clemens (variegated leafroller); P. stultana Walsingham (omnivorous leafroller); Spilonota ocellana Denis & Schiffermüller (eyespotted bud moth); and Suleima helianthana Riley (sunflower bud moth).

Selected other agronomic pests in the order Lepidoptera include, but are not limited to, Alsophila pometaria Harris (fall cankerworm); Anarsia lineatella Zeller (peach twig borer); Anisota senatoria J. E. Smith (orange striped oakworm); Antheraea pernyi Guérin-Méneville (Chinese Oak Silk moth); Bombyx mori Linnaeus (Silkworm); Bucculatrix thurberiella Busck (cotton leaf perforator); Colias eurytheme Boisduval (alfalfa caterpillar); Datana integerrima Grote & Robinson (walnut caterpillar); Dendrolimus sibiricus Tschetwerikov (Siberian silk moth), Ennomos subsignaria Hubner (elm spanworm); Erannis tiliaria Harris (linden looper); Erechthias flavistriata Walsingham (sugarcane bud moth); Euproctis chrysorrhoea Linnaeus (browntail moth); Harrisina americana Guérin-Méneville (grapeleaf skeletonizer); Heliothis subflexa Guenee; Hemileuca oliviae Cockrell (range caterpillar); Hyphantria cunea Drury (fall webworm); Keiferia lycopersicella Walsingham (tomato pinworm); Lambdina fiscellaria fiscellaria Hulst (Eastern hemlock looper); L. fiscellaria lugubrosa Hulst (Western hemlock looper); Leucoma salicis Linnaeus (satin moth); Lymantria dispar Linnaeus (gypsy moth); Malacosoma spp.; Manduca quinquemaculata Haworth (five spotted hawk moth, tomato hornworm); M. sexta Haworth (tomato hornworm, tobacco hornworm); Operophtera brumata Linnaeus (winter moth); Orgyia spp.; Paleacrita vernata Peck (spring cankerworm); Papilio cresphontes Cramer (giant swallowtail, orange dog); Phryganidia californica Packard (California oakworm); Phyllocnistis citrella Stainton (citrus leafminer); Phyllonorycter blancardella Fabricius (spotted tentiform leafminer); Pieris brassicae Linnaeus (large white butterfly); P. rapae Linnaeus (small white butterfly); P. napi Linnaeus (green veined white butterfly); Platyptilia carduidactyla Riley (artichoke plume moth); Plutella xylostella Linnaeus (diamondback moth); Pectinophora gossypiella Saunders (pink bollworm); Pontia protodice Boisduval & Leconte (Southern cabbageworm); Sabulodes aegrotata Guenee (omnivorous looper); Schizura concinna J. E. Smith (red humped caterpillar); Sitotroga cerealella Olivier (Angoumois grain moth); Telchin licus Drury (giant sugarcane borer); Thaumetopoea pityocampa Schiffermüller (pine processionary caterpillar); Tineola bisselliella Hummel (webbing clothesmoth); Tuta absoluta Meyrick (tomato leafminer) and Yponomeuta padella Linnaeus (ermine moth).

Of interest are larvae and adults of the order Coleoptera including weevils from the families Anthribidae, Bruchidae, and Curculionidae including, but not limited to: Anthonomus grandis Boheman (boll weevil); Cylindrocopturus adspersus LeConte (sunflower stem weevil); Diaprepes abbreviatus Linnaeus (Diaprepes root weevil); Hypera punctata Fabricius (clover leaf weevil); Lissorhoptrus oryzophilus Kuschel (rice water weevil); Metamasius hemipterus hemipterus Linnaeus (West Indian cane weevil); M. hemipterus sericeus Olivier (silky cane weevil); Sitophilus granarius Linnaeus (granary weevil); S. oryzae Linnaeus (rice weevil); Smicronyx fulvus LeConte (red sunflower seed weevil); S. sordidus LeConte (gray sunflower seed weevil); Sphenophorus maidis Chittenden (maize billbug); S. livis Vaurie (sugarcane weevil); Rhabdoscelus obscurus Boisduval (New Guinea sugarcane weevil); flea beetles, cucumber beetles, rootworms, leaf beetles, potato beetles, and leafminers in the family Chrysomelidae including, but not limited to: Chaetocnema ectypa Horn (desert corn flea beetle); C. pulicaria Melsheimer (corn flea beetle); Colaspis brunnea Fabricius (grape colaspis); Diabrotica barberi Smith & Lawrence (northern corn rootworm); D. undecimpunctata howardi Barber (southern corn rootworm); D. virgifera virgifera LeConte (western corn rootworm); Leptinotarsa decemlineata Say (Colorado potato beetle); Oulema melanopus Linnaeus (cereal leaf beetle); Phyllotreta cruciferae Goeze (corn flea beetle); Zygogramma exclamationis Fabricius (sunflower beetle); beetles from the family Coccinellidae including, but not limited to: Epilachna varivestis Mulsant (Mexican bean beetle); chafers and other beetles from the family Scarabaeidae including, but not limited to: Antitrogus parvulus Britton (Childers cane grub); Cyclocephala borealis Arrow (northern masked chafer, white grub); C. immaculata Olivier (southern masked chafer, white grub); Dermolepida albohirtum Waterhouse (Greyback cane beetle); Euetheola humilis rugiceps LeConte (sugarcane beetle); Lepidiota frenchi Blackburn (French's cane grub); Tomarus gibbosus De Geer (carrot beetle); T. subtropicus Blatchley (sugarcane grub); Phyllophaga crinita Burmeister (white grub); P. latifrons LeConte (June beetle); Popillia japonica Newman (Japanese beetle); Rhizotrogus majalis Razoumowsky (European chafer); carpet beetles from the family Dermestidae; wireworms from the family Elateridae, Eleodes spp., Melanotus spp. including M. communis Gyllenhal (wireworm); Conoderus spp.; Limonius spp.; Agriotes spp.; Ctenicera spp.; Aeolus spp.; bark beetles from the family Scolytidae; beetles from the family Tenebrionidae; beetles from the family Cerambycidae such as, but not limited to, Migdolus fryanus Westwood (longhorn beetle); and beetles from the Buprestidae family including, but not limited to, Aphanisticus cochinchinae seminulum Obenberger (leaf-mining buprestid beetle).

Adults and immatures of the order Diptera are of interest, including leafminers Agromyza parvicornis Loew (corn blotch leafminer); midges including, but not limited to: Contarinia sorghicola Coquillett (sorghum midge); Mayetiola destructor Say (Hessian fly); Neolasioptera murtfeldtiana Felt, (sunflower seed midge); Sitodiplosis mosellana Géhin (wheat midge); fruit flies (Tephritidae), Oscinella frit Linnaeus (frit flies); maggots including, but not limited to: Delia spp. including Delia platura Meigen (seedcorn maggot); D. coarctata Fallen (wheat bulb fly); Fannia canicularis Linnaeus, F. femoralis Stein (lesser house flies); Meromyza americana Fitch (wheat stem maggot); Musca domestica Linnaeus (house flies); Stomoxys calcitrans Linnaeus (stable flies)); face flies, horn flies, blow flies, Chrysomya spp.; Phormia spp.; and other muscoid fly pests, horse flies Tabanus spp.; bot flies Gastrophilus spp.; Oestrus spp.; cattle grubs Hypoderma spp.; deer flies Chrysops spp.; Melophagus ovinus Linnaeus (keds); and other Brachycera, mosquitoes Aedes spp.; Anopheles spp.; Culex spp.; black flies Prosimulium spp.; Simulium spp.; biting midges, sand flies, sciarids, and other Nematocera.

Included as insects of interest are those of the order Hemiptera such as, but not limited to, the following families: Adelgidae, Aleyrodidae, Aphididae, Asterolecaniidae, Cercopidae, Cicadellidae, Cicadidae, Cixiidae, Coccidae, Coreidae, Dactylopiidae, Delphacidae, Diaspididae, Eriococcidae, Flatidae, Fulgoridae, lssidae, Lygaeidae, Margarodidae, Membracidae, Miridae, Ortheziidae, Pentatomidae, Phoenicococcidae, Phylloxeridae, Pseudococcidae, Psyllidae, Pyrrhocoridae and Tingidae.

Agronomically important members from the order Hemiptera include, but are not limited to: Acrosternum hilare Say (green stink bug); Acyrthisiphon pisum Harris (pea aphid); Adelges spp. (adelgids); Adelphocoris rapidus Say (rapid plant bug); Anasa tristis De Geer (squash bug); Aphis craccivora Koch (cowpea aphid); A. fabae Scopoli (black bean aphid); A. gossypii Glover (cotton aphid, melon aphid); A. maidiradicis Forbes (corn root aphid); A. pomi De Geer (apple aphid); A. spiraecola Patch (spirea aphid); Aulacaspis tegalensis Zehntner (sugarcane scale); Aulacorthum solani Kaltenbach (foxglove aphid); Bemisia tabaci Gennadius (tobacco whitefly, sweetpotato whitefly); B. argentifolii Bellows & Perring (silverleaf whitefly); Blissus leucopterus leucopterus Say (chinch bug); Blostomatidae spp.; Brevicoryne brassicae Linnaeus (cabbage aphid); Cacopsylla pyricola Foerster (pear psylla); Calocoris norvegicus Gmelin (potato capsid bug); Chaetosiphon fragaefolii Cockerell (strawberry aphid); Cimicidae spp.; Coreidae spp.; Corythuca gossypii Fabricius (cotton lace bug); Cyrtopeltis modesta Distant (tomato bug); C. notatus Distant (suckfly); Deois flavopicta Stål (spittlebug); Dialeurodes citri Ashmead (citrus whitefly); Diaphnocoris chlorionis Say (honeylocust plant bug); Diuraphis noxia Kurdjumov/Mordvilko (Russian wheat aphid); Duplachionaspis divergens Green (armored scale); Dysaphis plantaginea Paaserini (rosy apple aphid); Dysdercus suturellus Herrich-Schaffer (cotton stainer); Dysmicoccus boninsis Kuwana (gray sugarcane mealybug); Empoasca fabae Harris (potato leafhopper); Eriosoma lanigerum Hausmann (woolly apple aphid); Erythroneoura spp. (grape leafhoppers); Eumetopina flavipes Muir (Island sugarcane planthopper); Eurygaster spp.; Euschistus servus Say (brown stink bug); E. variolarius Palisot de Beauvois (one-spotted stink bug); Graptostethus spp. (complex of seed bugs); and Hyalopterus pruni Geoffroy (mealy plum aphid); Icerya purchasi Maskell (cottony cushion scale); Labopidicola affii Knight (onion plant bug); Laodelphax striatellus Fallen (smaller brown planthopper); Leptoglossus corculus Say (leaf-footed pine seed bug); Leptodictya tabida Herrich-Schaeffer (sugarcane lace bug); Lipaphis erysimi Kaltenbach (turnip aphid); Lygocoris pabulinus Linnaeus (common green capsid); Lygus lineolaris Palisot de Beauvois (tarnished plant bug); L. Hesperus Knight (Western tarnished plant bug); L. pratensis Linnaeus (common meadow bug); L. rugulipennis Poppius (European tarnished plant bug); Macrosiphum euphorbiae Thomas (potato aphid); Macrosteles quadrilineatus Forbes (aster leafhopper); Magicicada septendecim Linnaeus (periodical cicada); Mahanarva fimbriolata Stål (sugarcane spittlebug); M. posticata Stål (little cicada of sugarcane); Melanaphis sacchari Zehntner (sugarcane aphid); Melanaspis glomerata Green (black scale); Metopolophium dirhodum Walker (rose grain aphid); Myzus persicae Sulzer (peach-potato aphid, green peach aphid); Nasonovia ribisnigri Mosley (lettuce aphid); Nephotettix cinticeps Uhler (green leafhopper); N. nigropictus Stål (rice leafhopper); Nezara viridula Linnaeus (southern green stink bug); Nilaparvata lugens Stål (brown planthopper); Nysius ericae Schilling (false chinch bug); Nysius raphanus Howard (false chinch bug); Oebalus pugnax Fabricius (rice stink bug); Oncopeltus fasciatus Dallas (large milkweed bug); Orthops campestris Linnaeus; Pemphigus spp. (root aphids and gall aphids); Peregrinus maidis Ashmead (corn planthopper); Perkinsiella saccharicida Kirkaldy (sugarcane delphacid); Phylloxera devastatrix Pergande (pecan phylloxera); Planococcus citri Risso (citrus mealybug); Plesiocoris rugicollis Fallen (apple capsid); Poecilocapsus lineatus Fabricius (four-lined plant bug); Pseudatomoscelis seriatus Reuter (cotton fleahopper); Pseudococcus spp. (other mealybug complex); Pulvinaria elongata Newstead (cottony grass scale); Pyrilla perpusilla Walker (sugarcane leafhopper); Pyrrhocoridae spp.; Quadraspidiotus perniciosus Comstock (San Jose scale); Reduviidae spp.; Rhopalosiphum maidis Fitch (corn leaf aphid); R. padi Linnaeus (bird cherry-oat aphid); Saccharicoccus sacchari Cockerell (pink sugarcane mealybug); Scaptocoris castanea Perty (brown root stink bug); Schizaphis graminum Rondani (greenbug); Sipha flava Forbes (yellow sugarcane aphid); Sitobion avenae Fabricius (English grain aphid); Sogatella furcifera Horvath (white-backed planthopper); Sogatodes oryzicola Muir (rice delphacid); Spanagonicus albofasciatus Reuter (whitemarked fleahopper); Therioaphis maculata Buckton (spotted alfalfa aphid); Tinidae spp.; Toxoptera aurantii Boyer de Fonscolombe (black citrus aphid); and T. citricida Kirkaldy (brown citrus aphid); Trialeurodes abutiloneus (bandedwinged whitefly) and T. vaporariorum Westwood (greenhouse whitefly); Trioza diospyri Ashmead (persimmon psylla); and Typhlocyba pomaria McAtee (white apple leafhopper).

Also included are adults and larvae of the order Acari (mites) such as Aceria tosichella Keifer (wheat curl mite); Panonychus ulmi Koch (European red mite); Petrobia latens Müller (brown wheat mite); Steneotarsonemus bancrofti Michael (sugarcane stalk mite); spider mites and red mites in the family Tetranychidae, Oligonychus grypus Baker & Pritchard, O. indicus Hirst (sugarcane leaf mite), O. pratensis Banks (Banks grass mite), O. stickneyi McGregor (sugarcane spider mite); Tetranychus urticae Koch (two spotted spider mite); T. mcdanieli McGregor (McDaniel mite); T. cinnabarinus Boisduval (carmine spider mite); T. turkestani Ugarov & Nikolski (strawberry spider mite), flat mites in the family Tenuipalpidae, Brevipalpus lewisi McGregor (citrus flat mite); rust and bud mites in the family Eriophyidae and other foliar feeding mites and mites important in human and animal health, i.e. dust mites in the family Epidermoptidae, follicle mites in the family Demodicidae, grain mites in the family Glycyphagidae, ticks in the order Ixodidae. Ixodes scapularis Say (deer tick); I. holocyclus Neumann (Australian paralysis tick); Dermacentor variabilis Say (American dog tick); Amblyomma americanum Linnaeus (lone star tick); and scab and itch mites in the families Psoroptidae, Pyemotidae, and Sarcoptidae.

Insect pests of the order Thysanura are of interest, such as Lepisma saccharina Linnaeus (silverfish); Thermobia domestica Packard (firebrat).

Additional arthropod pests covered include: spiders in the order Araneae such as Loxosceles recluse Gertsch & Mulaik (brown recluse spider); and the Latrodectus mactans Fabricius (black widow spider); and centipedes in the order Scutigeromorpha such as Scutigera coleoptrata Linnaeus (house centipede). In addition, insect pests of the order Isoptera are of interest, including those of the Termitidae family, such as, but not limited to, Cornitermes cumulans Kollar, Cylindrotermes nordenskioeldi Holmgren and Pseudacanthotermes militaris Hagen (sugarcane termite); as well as those in the Rhinotermitidae family including, but not limited to Heterotermes tenuis Hagen. Insects of the order Thysanoptera are also of interest, including but not limited to thrips, such as Stenchaetothrips minutus van Deventer (sugarcane thrips).

Embodiments of the present disclosure are further defined in the following Examples. It should be understood that these Examples are given by way of illustration only. From the above discussion and these Examples, one skilled in the art can ascertain the essential characteristics of this disclosure, and without departing from the spirit and scope thereof, can make various changes and modifications of the embodiments of the disclosure to adapt it to various usages and conditions. Thus, various modifications of the embodiments of the disclosure, in addition to those shown and described herein, will be apparent to those skilled in the art from the foregoing description. Such modifications are also intended to fall within the scope of the appended claims.

The disclosure of each reference set forth herein is incorporated by reference in its entirety.

### EXAMPLES

### Example 1

### Transformation of Maize by Agrobacterium Transformation and Regeneration of Transgenic Plants Containing the vip3Aa20, cry2A.127, cry1A.88, and Mo-Pat Genes

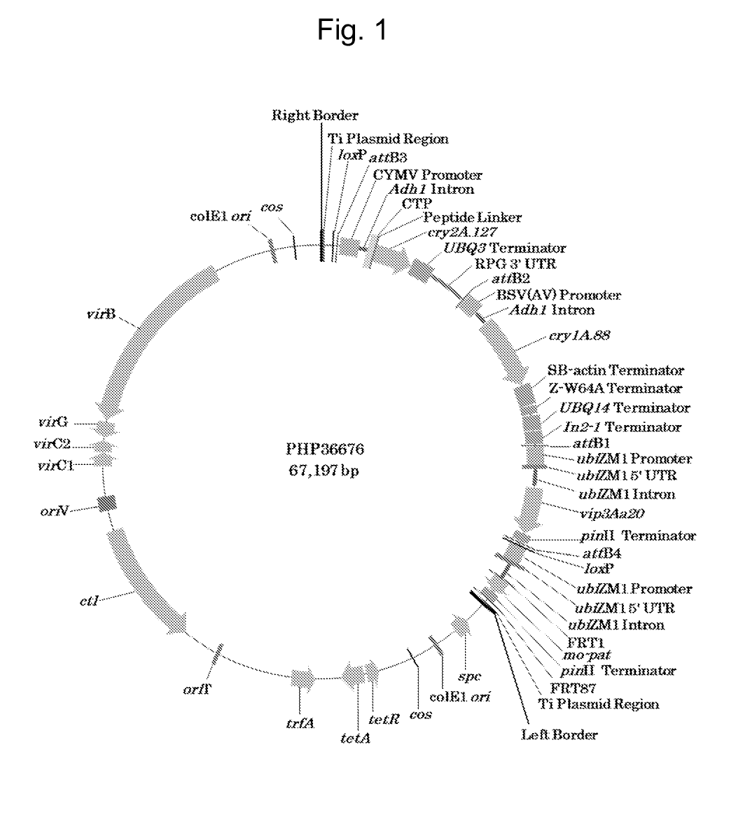

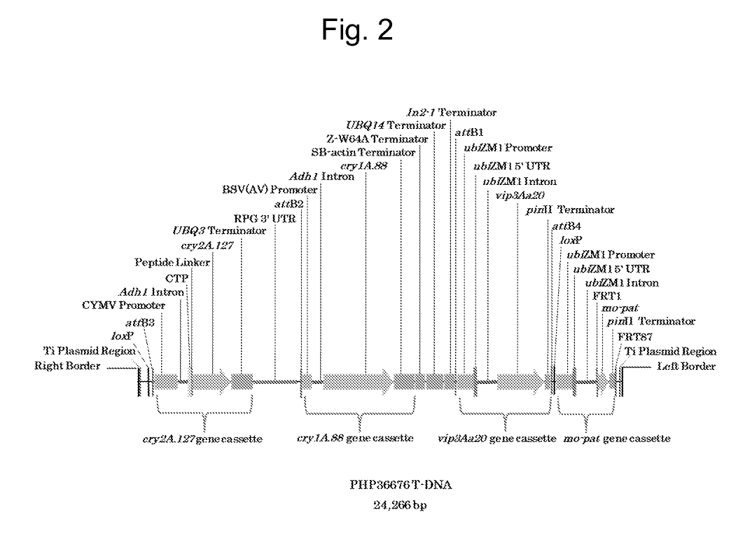

Maize (Zea mays L.) was transformed by Agrobacterium-mediated transformation with plasmid PHP36676 (FIG. 1). The T-DNA region of this plasmid is represented schematically in FIG. 2 and sequence is set forth in SEQ ID NO: 1. A summary of the genetic elements and their positions on plasmid PHP36676 and on the T-DNA is described in Tables 2 and 3, respectively.

The T-DNA of plasmid PHP36676 contains four gene cassettes. The first cassette (cry2A.127 gene cassette) contains the cry2A.127 gene encoding the Cry2A.127 protein that has been functionally optimized using DNA shuffling techniques and based on genes derived from Bacillus thuringiensis subsp. kurstaki. The 634-residue protein produced by expression of the cry2A.127 sequence is targeted to maize chloroplasts through the addition of a 54-amino acid chloroplast transit peptide (CTP) (US Patent No. U.S. Pat. No. 7,563,863B2) as well as a 6-amino acid linker (Peptide Linker) resulting in a total length of 694 amino acids (approximately 77 kDa) for the precursor protein (the CTP sequence is cleaved upon insertion into the chloroplast, resulting in a mature protein of 644 amino acids in length with an approximate molecular weight of 72 kDa; (SEQ ID NO: 17). The expression of the cry2A.127 gene and the CTP is controlled by the promoter from the Citrus Yellow Mosaic Virus (CYMV) (Huang and Hartung, 2001, Journal of General Virology 82: 2549-2558; Genbank accession NC_{—}003382.1) along with the intron 1 region from maize alcohol dehydrogenase gene (Adh1 Intron) (Dennis et al., 1984, Nucleic Acids Research 12: 3983-4000). Transcription of the cry2A.127 gene cassette is terminated by the presence of the terminator from the ubiquitin 3 (UBQ3) gene of Arabidopsis thaliana (Callis et al., 1995, Genetics 139: 921-939). In addition, a genomic fragment corresponding to the 3′ untranslated region from a ribosomal protein gene (RPG 3′ UTR) of Arabidopsis thaliana (Salanoubat et al., 2000, Nature 408: 820-822; TAIR accession AT3G28500) is located between the cry2A.127 and cry1A.88 cassettes in order to prevent any potential transcriptional interference with downstream cassettes. Transcriptional interference is defined as the transcriptional suppression of one gene on another when both are in close proximity (Shearwin, et al., 2005, Trends in Genetics 21: 339-345). The presence of a transcriptional terminator between two cassettes has been shown to reduce the occurrence of transcriptional interference (Greger et al., 1998, Nucleic Acids Research 26: 1294-1300); the placement of multiple terminators between cassettes is intended to prevent this effect.

The second cassette (cry1A.88 gene cassette) contains a second shuffled insect control gene, cry1A.88, encoding the Cry1A.88 protein that has been functionally optimized using DNA shuffling techniques and based on genes derived from Bacillus thuringiensis subsp. kurstaki. The coding region which produces a 1,182-residue protein (approximately 134 kDa; SEQ ID NO: 18) is controlled by a truncated version of the promoter from Banana Streak Virus of acuminata Vietnam strain [BSV (AV)] (Lheureux et al., 2007, Archives of Virology 152: 1409-1416; Genbank accession NC_{—}007003.1) with a second copy of the maize Adh1 intron. The terminator for the cry1A.88 cassette is a portion of the Sorghum bicolor genome containing the terminator from the actin gene (SB-actin) (Genbank accession XM_{—}002441128.1).

Three additional terminators are present between the second and third cassettes: the terminator from the 27 kDa zein gene of maize W64A line (Z-W64A) (Das et al., 1991, Genomics 11: 849-856), a genomic fragment of Arabidopsis thaliana chromosome 4 containing the ubiquitin 14 (UBQ14) terminator (Callis et al., 1995, Genetics 139: 921-939), and the terminator sequence from the maize Int-1 gene (Hershey and Stoner, 1991, Plant Molecular Biology 17: 679-690). These additional elements are intended to prevent any potential transcriptional interference with the downstream cassettes.

The third cassette (vip3Aa20 gene cassette) contains the modified vip3A gene derived from Bacillus thuringiensis strain AB88, which encodes the insecticidal Vip3Aa20 protein (Estruch et al., 1996, PNAS 93: 5389-5394). Expression of the vip3Aa20 gene is controlled by the regulatory region of the maize polyubiquitin (ubiZM1) gene, including the promoter, the 5′ untranslated region (5′ UTR) and intron (Christensen et al., 1992, Plant Molecular Biology 18: 675-689). The terminator for the vip3Aa20 gene is the terminator sequence from the proteinase inhibitor II (pinII) gene of Solanum tuberosum (Keil et al., 1986, Nucleic Acids Research 14: 5641-5650; An et al., 1989, The Plant Cell 1: 115-122). The Vip3Aa20 protein is 789-amino acid residues in length with an approximate molecular weight of 88 kDa (SEQ ID NO: 19).

The fourth gene cassette (mo-pat gene cassette) contains a maize-optimized version of the phosphinothricin acetyl transferase gene (mo-pat) from Streptomyces viridochromogenes (Wohlleben et al., 1988, Gene 70: 25-37). The mo-pat gene expresses the phosphinothricin acetyl transferase (PAT) enzyme that confers tolerance to phosphinothricin. The PAT protein is 183 amino acids in length and has an approximate molecular weight of 21 kDa (SEQ ID NO: 20). Expression of the mo-pat gene is controlled by a second copy of the ubiZM1 promoter, the 5′ UTR and intron (Christensen et al., 1992, Plant Molecular Biology 18: 675-689), in conjunction with a second copy of the pinII terminator (Keil et al., 1986, Nucleic Acids Research 14: 5641-5650; An et al., 1989, The Plant Cell 1: 115-122).

The PHP36676 T-DNA contains two Flp recombinase target sequences (FRT1 and FRT87 sites) as well as two loxP and four attB recombination sites (Proteau et al., 1986, Nucleic Acids Research 14: 4787-4802; Dale and Ow, 1990, Gene 91: 79-85; Hartley et al., 2000, Genome Research 10: 1788-1795; Cheo et al., 2004, Genome Research 14: 2111-2120; WO 2007/011733). The presence of these sites alone does not cause any recombination, since in order to function, these sites need a specific recombinase enzyme that is not naturally present in plants (Cox, 1988, American Society for Microbiology, pp 429-443; Dale and Ow, 1990, Gene 91: 79-85; Thorpe et al., 1998, PNAS 95: 5505-5510).

TABLE 2

| | | Known | Size | |
|---|---|---|---|---|
| | Location on plasmid | Genetic | (base | |
| Region | (base pair position) | Element | pairs) | Description |
| T-DNA | 1-24,266 | | 24,266 | See Table 2 for information on the |
| | | | | elements in this region |
| Plasmid | 24,267-49,149 | includes | 24,883 | DNA from various sources for plasmid |
| Construct | | elements | | construction and plasmid replication |
| | | below | | |
| | 25,442-26,230 | spc | 789 | Spectinomycin resistance gene from bacteria |
| | (complementary) | | | (Fling et al., 1985) |
| | 27,353-27,722 | colE1 ori | 370 | Bacterial origin of replication region (E. coli) |
| | | | | (Tomizawa et al., 1977) |
| | 28,819-28,832 | cos | 14 | cos site; cohesive ends from lambda |
| | | | | bacteriophage DNA (Komari et al., 1996) |
| | 30,533-31,183 | tetR | 651 | Tetracycline resistance regulation gene |
| | (complementary) | | | from bacteria (Komari et al., 1996) |
| | 31,289-32,488 | tetA | 1,200 | Tetracycline resistance gene from bacteria |
| | | | | (Komari et al., 1996) |
| | 33,119-35,308 | rep | 2,190 | rep operon from bacteria (includes trfA |
| | (complementary) | | | below) (Komari et al., 1996) |
| | 33,761-34,909 | trfA | 1,149 | Trans-acting replication gene from |
| | (complementary) | | | bacteria (Komari et al., 1996) |
| | 38,723-38,834 | oriT | 112 | oriT origin of transfer region from bacteria |
| | | | | (Komari et al., 1996) |
| | 40,674-46,944 | ctl | 6,271 | Central control operon region from |
| | (complementary) | | | bacteria (Komari et al., 1996) |
| | 47,952-48,662 | oriV | 711 | oriV origin of replication region from |
| | | | | bacteria (Komari et al., 1996) |
| Ti | 49,150-63,966 | includes | 14,817 | Virulence (vir) gene region and intergenic |
| Plasmid | | elements | | regions from Ti plasmid of Agrobacterium |
| Backbone | | below | | tumefaciens (Komari et al., 1996) |
| | 50,175-50,869 | virC1 | 695 | Virulence gene important for T-DNA |
| | | | | insertion into genome |
| | 50,872-51,480 | virC2 | 609 | Virulence gene important for T-DNA |
| | | | | insertion into genome |
| | 51,591-52,394 | virG | 804 | Virulence gene important for T-DNA |
| | (complementary) | | | insertion into genome |
| | 52,526-61,961 | virB | 9,436 | Virulence gene important for T-DNA |
| | (complementary) | | | insertion into genome |
| Plasmid | 63,967-67,197 | includes | 3,231 | DNA from various sources for plasmid |
| Construct | | elements | | construction and plasmid replication |
| | | below | | |
| | 64,262-64,631 | colEl ori | 370 | Bacterial origin of replication region (E. coli) |
| | | | | (Tomizawa et al., 1977) |
| | 65,724-65,737 | cos | 14 | cos site; cohesive ends from lambda |
| | | | | bacteriophage DNA (Komari et al., 1996) |

TABLE 3

| | Location on T- | | Size | |
|---|---|---|---|---|
| | DNA (base | | (base | |
| | pair position) | Genetic Element | pairs) | Description |
| | 1-25 | Right Border | 25 | T-DNA Right Border region from the Ti |
| | | | | plasmid of Agrobacterium tumefaciens strain C58 |
| | 26-177 | Ti Plasmid Region | 152 | Non-functional sequence from the Ti plasmid |
| | | | | of Agrobacterium tumefaciens strain C58 |
| | 178-435 | Intervening | 258 | DNA sequence used for cloning |
| | | Sequence | | |
| | 436-469 | loxP | 34 | Bacteriophage P1 recombination site recognized |
| | | | | by Cre recombinase (Dale and Ow, 1990) |
| | 470-696 | Intervening | 227 | DNA sequence used for cloning |
| | | Sequence | | |
| | 697-717 | attB3 | 21 | Bacteriophage lambda integrase recombination |
| | | | | site (Cheo et al., 2004) |
| | 718-758 | Intervening | 41 | DNA sequence used for cloning |
| | | Sequence | | |
| cry2A.127 | 759-1,911 | CYMV Promoter | 1,153 | Promoter from Citrus Yellow Mosaic Virus |
| gene | | | | (CYMV) (Huang and Hartung, 2001; Genbank |
| cassette | | | | accession NC_003382.1) |
| | 1,912-1,938 | Intervening | 27 | DNA sequence used for cloning |
| | | Sequence | | |
| | 1,939-2,481 | Adh1 Intron | 543 | Intron 1 region from the alcohol dehydrogenase |
| | | | | gene of Zea mays (Dennis et al., 1984) |
| | 2,482-2,495 | Intervening | 14 | DNA sequence used for cloning |
| | | Sequence | | |
| | 2,496-2,657 | CTP | 162 | Sequence encoding chloroplast transit peptide |
| | | | | that transports target protein from cytoplasm to |
| | | | | chloroplast (Lassner and Wilkinson, 2009; U.S. |
| | | | | Pat. No. 7,563,863, B2) |
| | 2,658-2,675 | Peptide Linker | 18 | Six amino acid “linker” sequence |
| | 2,676-4,580 | cry2A.127 | 1,905 | Gene encoding the Cry2A.127 protein, derived |
| | | | | from a naturally occurring Bacillus thuringiensis |
| | | | | subsp. kurstaki gene that confers protection |
| | | | | from certain lepidopteran pests |
| | 4,581-4,610 | Intervening | 30 | DNA sequence used for cloning |
| | | Sequence | | |
| | 4,611-5,699 | UBQ3 Terminator | 1,089 | Terminator from the ubiquitin 3 (UBQ3) gene |
| | | | | of Arabidopsis thaliana (Callis et al., 1995) |
| | 5,700-5,704 | Intervening | 5 | DNA sequence used for cloning |
| | | Sequence | | |
| | 5,705-7,932 | RPG 3′ UTR | 2,228 | 3′ untranslated region from a ribosomal protein |
| | | | | gene of Arabidopsis thaliana (Salanoubat et al., |
| | | | | 2000; TAIR accession AT3G28500) |
| | 7,933-8,095 | Intervening | 163 | DNA sequence used for cloning |
| | | Sequence | | |
| | 8,096-8,119 | attB2 | 24 | Bacteriophage lambda integrase recombination |
| | | | | site (Hartley et al., 2000) |
| | 8,120-8,182 | Intervening | 63 | DNA sequence used for cloning |
| | | Sequence | | |
| cry1A.88 | 8,183-8,652 | BSV (AV) | 470 | Promoter derived from Banana Streak Virus of |
| gene | | Promoter | | acuminata Vietnam strain [BSV (AV)] |
| cassette | | | | (Lheureux et al., 2007; Genbank accession |
| | | | | NC_307003.1) |
| | 8,653-8,679 | Intervening | 27 | DNA sequence used for cloning |
| | | Sequence | | |
| | 8,680-9,222 | Adh1 Intron | 543 | Intron 1 region from the alcohol dehydrogenase |
| | | | | gene of Zea mays (Dennis et al., 1984) |
| | 9,223-9,236 | Intervening | 14 | DNA sequence used for cloning |
| | | Sequence | | |
| | 9,237-12,785 | cry1A.88 | 3,549 | Gene encoding the Cry1A.88 protein, derived |
| | | | | from a naturally occurring Bacillus thuringiensis |
| | | | | subsp. kurstaki gene that confers protection |
| | | | | from certain lepidopteran pests |
| | 12,786-12,803 | Intervening | 18 | DNA sequence used for cloning |
| | | Sequence | | |
| | 12,804-13,846 | SB-actin | 1,043 | Terminator from the actin gene of Sorghum |
| | | Terminator | | bicolor (Genbank accession XM_002441128.1) |
| | 13,847-13,879 | Intervening | 33 | DNA sequence used for cloning |
| | | Sequence | | |
| | 13,880-14,359 | Z-W64A | 480 | Terminator from the 27 kDa zein gene of Zea |
| | | Terminator | | mays W64A line (Das et al., 1991) |
| | 14,360-14,365 | Intervening | 6 | DNA sequence used for cloning |
| | | Sequence | | |
| | 14,366-15,267 | UBQ14 Terminator | 902 | Terminator from the ubiquitin 14 (UBQ14) gene |
| | | | | of Arabidopsis thaliana (Callis et al., 1995) |
| | 15,268-15,273 | Intervening | 6 | DNA sequence used for cloning |
| | | Sequence | | |
| | 15,274-15,767 | In2-1 Terminator | 494 | Terminator from the In2-1 gene of Zea mays |
| | | | | (Hershey and Stoner, 1991) |
| | 15,768-15,856 | Intervening | 89 | DNA sequence used for cloning |
| | | Sequence | | |
| | 15,857-15,880 | attB1 | 24 | Bacteriophage lambda integrase recombination |
| | | | | site (Hartley et al., 2000) |
| | 15,881-15,963 | Intervening | 83 | DNA sequence used for cloning |
| | | Sequence | | |
| vip3Aa20 | 15,964-16,863 | ubiZM1 Promoter | 900 | Promoter region from the polyubiquitin gene of |
| gene | | | | Zea mays (Christensen et al., 1992) |
| cassette | 16,864-16,946 | ubiZM1 5′ UTR | 83 | 5′ untranslated region from the polyubiquitin |
| | | | | gene of Zea mays (Christensen et al., 1992) |
| | 16,947-17,959 | ubiZM1 Intron | 1,013 | Intron region from the polyubiquitin gene of |
| | | | | Zea mays (Christensen et al., 1992) |
| | 17,960-17,985 | Intervening | 26 | DNA sequence used for cloning |
| | | Sequence | | |
| | 17,986-20,355 | vip3Aa20 | 2,370 | Modified vip3A gene derived from Bacillus |
| | | | | thuringiensis strain AB88 (Estruch et al., 1996) |
| | 20,356-20,361 | Intervening | 6 | DNA sequence used for cloning |
| | | Sequence | | |
| | 20,362-20,671 | pinII Terminator | 310 | Terminator from the proteinase inhibitor II |
| | | | | gene of Solarium tuberosum (Keil et al., 1986; An et |
| | | | | al., 1989) |
| | 20,672-20,791 | Intervening | 120 | DNA sequence used for cloning |
| | | Sequence | | |
| | 20,792-20,812 | attB4 | 21 | Bacteriophage lambda integrase recombination |
| | | | | site (Cheo et al., 2004) |
| | 20,813-20,887 | Intervening | 75 | DNA sequence used for cloning |
| | | Sequence | | |
| | 20,888-20,921 | loxP | 34 | Bacteriophage P1 recombination site recognized |
| | | | | by Cre recombinase (Dale and Ow, 1990) |
| | 20,922-20,940 | Intervening | 19 | DNA sequence used for cloning |
| | | Sequence | | |
| mo-pat | 20,941-21,840 | ubiZM1 Promoter | 900 | Promoter region from the polyubiquitin gene of |
| gene | | | | Zea mays (Christensen et al., 1992) |
| cassette | 21,841-21,923 | ubiZM1 5′ UTR | 83 | 5′ untranslated region from the polyubiquitin |
| | | | | gene of Zea mays (Christensen et al., 1992) |
| | 21,924-22,936 | ubiZM1 Intron | 1,013 | Intron region from the polyubiquitin gene of |
| | | | | Zea mays (Christensen et al., 1992) |
| | 22,937-22,964 | Intervening | 28 | DNA sequence used for cloning |
| | | Sequence | | |
| | 22,965-23,012 | FRT1 | 48 | Flp recombinase DNA binding site from |
| | | | | Saccharomyces cerevisiae (Proteau et al., 1986) |
| | 23,013-23,038 | Intervening | 26 | DNA sequence used for cloning |
| | | Sequence | | |
| | 23,039-23,590 | mo-pat | 552 | Maize-optimized gene encoding the |
| | | | | phosphinothricin acetyltransferase protein |
| | | | | (PAT), derived from Streptomyces viridochromogenes |
| | | | | (Wohlleben et al., 1988) |
| | 23,591-23,598 | Intervening | 8 | DNA sequence used for cloning |
| | | Sequence | | |
| | 23,599-23,908 | pinII Terminator | 310 | Terminator from the proteinase inhibitor II |
| | | | | gene of Solarium tuberosum (Keil et al., 1986; An et |
| | | | | al., 1989) |
| | 23,909-23,929 | Intervening | 21 | DNA sequence used for cloning |
| | | Sequence | | |
| | 23,930-23,977 | FRT87 | 48 | Modified Flp recombinase DNA binding site |
| | | | | derived from Saccharomyces cerevisiae FRT site |
| | | | | (Tao et al., 2007) |
| | 23,978-24,184 | Intervening | 207 | DNA sequence used for cloning |
| | | Sequence | | |
| | 24,185-24,241 | Ti Plasmid Region | 57 | Non-functional sequence from the Ti plasmid |
| | | | | of Agrobacterium tumefaciens strain C58 |
| | 24,242-24,266 | Left Border | 25 | T-DNA Left Border region from the Ti plasmid |
| | | | | of Agrobacterium tumefaciens strain C58 |

Immature embryos of maize (Zea mays L.) were aseptically removed from the developing caryopsis nine to eleven days after pollination and inoculated with Agrobacterium tumefaciens strain LBA4404 containing plasmid PHP36676, essentially as described in Zhao et al. (2001 Plant Cell Culture Protocols 318: 315-323). The T-DNA region of PHP36676 was inserted into the 033121 maize event. After three to six days of embryo and Agrobacterium co-cultivation on solid culture medium with no selection, the embryos were then transferred to a medium without herbicide selection but containing carbenicillin for selection against Agrobacterium. After three to five days on this medium, embryos were then transferred to selective medium that was stimulatory to maize somatic embryogenesis and contained bialaphos for selection of cells expressing the mo-pat transgene. The medium also contained carbenicillin select against any remaining Agrobacterium. After six to eight weeks on the selective medium, healthy, growing calli that demonstrated resistance to bialaphos were identified. The putative transgenic calli were subsequently regenerated to produce T0 plantlets.

PCR analysis was conducted on samples taken from the T0 plantlets for the presence of a single copy cry1A.88, cry2A.127, mo-pat and vip3Aa20 transgenes from the PHP36676 T-DNA and the absence of certain Agrobacterium binary vector backbone sequences by PCR. Plants that were determined to be single copy for the inserted genes and negative for vector backbone sequences were selected for further greenhouse propagation and trait efficacy confirmation. The T0 plants with a single copy of the T-DNA and meeting the trait efficacy criteria, including 033121 maize, were advanced and crossed to inbred lines to produce seed for further testing.

### Example 2

### Identification of Maize Event DP-033121-3

Genomic DNA from leaf tissue of the test seeds from 33121 maize and the control seeds from a non-genetically modified maize line with a genetic background representative of the test seed was isolated and subjected to qualitative PCR amplification using a construct-specific primer pair. The PCR products were separated on an agarose gel to confirm the presence of the inserted construct in the genomic DNA isolated from the test plants, and the absence of the inserted construct in the genomic DNA isolated from the control plants. The size of PCR products were estimated based on the molecular weight markers, PCR Markers (Catalog # G3161, Promega™, Madison, Wis.). The sensitivity of the construct-specific PCR assay was determined by detecting the amplification of the target PCR products from the 33121 maize DNA at various diluted amount in a total of 50-ng maize genomic DNA. The reliability of the PCR method was assessed by performing the PCR run three times.

Test and control leaf samples were harvested from plants grown at the DuPont Experimental Station (Wilmington, Del.) from seeds obtained from Pioneer Hi-Bred International, Inc., A DuPont Company (Johnston, Iowa). Genomic DNA was isolated using a urea extraction procedure following standard operating procedures and quantified using a fluorescence-based Quant-It™ PicoGreen® reagent kit (Catalog # P7589, Invitrogen™, Carlsbad, Calif.).

Genomic DNA samples isolated from leaf tissues of five 33121 maize and five control plants were subjected to PCR amplification using AmpliTaq Gold® PCR Master Mix (Catalog #4326717, Applied Biosystems™, Foster City, Calif.) in the presence of the construct-specific primer pair (12-O-4328/12-O-4327—SEQ ID NO: 2/SEQ ID NO: 3) which spans the junction of the cry1A.88 gene and SB-Actin terminator, and allows for the unique identification of the PHP36676 T-DNA inserted in 33121 maize. A second primer pair (12-O-4331/12-O-4332—SEQ ID NO: 4/SEQ ID NO: 5) to amplify the maize invertase gene (GenBank accession number AF171874.1) was used as the endogenous control for PCR amplification. Each PCR reaction was set up in a total volume of 50 μL with 50 ng of the isolated genomic DNA in the presence of appropriate primer pair at 0.4 μM and PCR reagents. 5-ng aliquot of PHP36676 plasmid DNA was used as the positive control for the construct-specific PCR, and ddH2O (no-template control) was used as a negative control in all PCR runs. The PCR target site for each primer pair and the sizes of the expected PCR amplicons are shown in Table 4. PCR reaction constituents and cycling program are shown in Table 5.

TABLE 4

| | | Expected Size of |
|---|---|---|
| Primer Pair | Target Site | PCR Amplicon (bp) |
| 12-O-4328/12-O-4327 | Spanning the junction of the cry1A.88 | 270 |
| | gene and SB-Actin terminator | |
| 12-O-4331/12-O-4332 | Maize endogenous invertase gene | 225 |

TABLE 5

| PCR Reaction Constituents | | PCR Cycling Program | | | | |
|---|---|---|---|---|---|---|
| | Volume | | | | | # |
| Component | (μL) | Cycle Step | Temp | Time | | Cycles |
| Template DNA¹ | 2 | Initial Denaturation/ | 95° C. | 5 | min | 1 |
| | | Enzyme Activation | | | | |
| Primer Pair (5 μM)² | 4 | Denaturation | 95° C. | 15 | sec | 35 |
| 2X PCR Master | 25 | Annealing/ | 65° C. | 30 | sec | |
| Mix³ | | Extension | | | | |
| ddH₂O⁴ | 19 | Final Extension | 72° C. | 7 | min | 1 |

| | | | | |
|---|---|---|---|---|
| Total | 50 | Hold Cycle | 4° C. | Until |
| | | | | Analysis |

¹Plant genomic DNA (25 ng/μL) or PHP36676 Plasmid DNA (2.5 ng/μL)
²5 μM of each primer
³ABI AmpliTaq Gold PCR Master Mix
⁴Double-distilled water

### Construct-Specific PCR Analysis for 33121 Maize

A PCR product of approximately 250 base pair (bp) was amplified and observed in five 33121 maize and five control maize plants using maize invertase gene-specific primer pair. This endogenous target band was not observed in PCR samples with no-template control or PHP36676 plasmid DNA. These results correspond closely with the expected PCR amplicon size (225 bp). This assay was performed a total of three times and the same results were obtained each time.

A PCR product of approximately 300 bp amplified by the construct-specific primer pair was observed in PCR samples with PHP36676 plasmid DNA and five of 33121 maize DNA samples, but was absent in five of control maize DNA samples and the no-template control. These results correspond closely with the expected PCR amplicon size (270 bp). This assay was performed a total of three times and the same results were obtained each time.

### Sensitivity of Construct-Specific PCR Analysis for 33121 Maize

In order to assess the sensitivity of the construct-specific PCR assay, 33121 maize DNA was diluted in control maize genomic DNA, resulting in test samples containing various amounts of 33121 maize DNA (50 ng, 5 ng, 1 ng, 200 pg, 100 pg, 50 pg, 20 pg, 10 pg, 5 pg and 1 pg) in a total of 50-ng maize DNA. These various amounts of 33121 maize DNA correspond to 100%, 10%, 2%, 0.4%, 0.2%, 0.1%, 0.04%, 0.02%, 0.01% and 0.002% of 33121 maize DNA in total maize genomic DNA. These various amounts of 33121 maize DNA were subjected to PCR amplification as previously conducted. Based on this analysis, the limit of detection (LOD) was determined to be approximately 20 pg of 33121 maize DNA in 50 ng of total DNA, or 0.04% of 33121 maize DNA. The sensitivity of this PCR detection method described is sufficient for many screening applications. This sensitivity testing was performed a total of three times and the same results were obtained each time.

Qualitative gel-based PCR analysis of the 33121 maize using a construct-specific primer pair confirmed that the test plants contained the inserted T-DNA region of plasmid PHP36676, as demonstrated by the presence of the target band in all test plants analyzed and its absence in the non-genetically modified control plants. The results were reproducible among three PCR runs. The maize endogenous reference gene assay for detecting the invertase gene amplified the expected size of PCR products from both test and control plants. The sensitivity of the PCR method under the conditions performed has demonstrated that this assay is able to detect approximately 20 pg of the 33121 maize DNA in a total of 50-ng maize genomic DNA, which is equivalent to 0.04% of the 33121 maize genomic DNA.

### Example 3

### Southern Blot Analysis of Maize Event DP-033121-3

Frozen leaf tissues were obtained from event DP-033121-3, which was generated by transforming a maize line with plasmid PHP36676. Eight plants from the S1 generation of event DP 033121-3 and untransformed control maize plants from the same genetic background were used for Southern blot analysis. Genomic DNA was extracted from frozen leaf tissue from each test and control plant using a urea extraction method. Genomic DNA extractions from individual plants were obtained and used for restriction digestion.

Genomic DNA samples from event DP-033121-3 were digested with Sca I for copy number analysis of the cry2A.127 gene, and Nco I for copy number analysis of the cry1A.88, vip3Aa20, and mo-pat genes. Plasmid PHP36676 was used as a positive control and genomic DNA from the near-isoline maize line was used as a negative control.

The cry2A.127 probe was used on Sca I digestion blots to provide copy number information of the inserts in event DP-033121-3. After Southern blot analysis, a single band of greater than 10,179 bp with the cry2A.127 probe denotes a single copy of the gene. Nco I digestion was used with the cry1A.88, vip3Aa20, and mo-pat probes to determine copy number of these genes. After Southern blot analysis, a single band of greater than 15,032 bp with the cry1A.88, vip3Aa20, and mo-pat probes indicates a single copy of each gene.

Following electrophoresis, agarose gels containing the separated DNA fragments were depurinated, denatured, and neutralized in situ. The DNA fragments were transferred to a nylon membrane in 20×SSC buffer using the method as described for the TURBOBLOTTER™ Rapid Downward Transfer System (Whatman, Inc.). Following transfer to the membrane, the DNA was bound to the membrane by UV crosslinking.

Probes homologous to the cry2A.127, cry1A.88, vip3Aa20, and mo-pat genes on plasmid PHP36676 were used for hybridization to confirm the presence of the genes. The probes were labeled by a PCR reaction incorporating a digoxigenin (DIG) labeled nucleotide, [DIG-11]-dUTP. PCR labeling of the probes was carried out according to the procedures supplied in the PCR DIG Probe Synthesis Kit (Roche). The labeled probes were hybridized to the target DNA on the blots for detection of the specific fragments using the DIG Easy Hyb Solution essentially as described by the manufacturer (Roche). Washes after hybridization were carried out at high stringency. The blot was visualized using the CDP-Star Chemiluminescent Nucleic Acid Detection System (Roche) in a Chemiluminiscent reader (GE Healthcare). Prior to hybridization with additional probes, membranes were stripped of hybridized probes following the manufacturer's recommendation.

Integration and copy number of the insertion were determined in event DP-033121-3 derived from construct PHP36676. A schematic map of the PHP36676 plasmid used in Agrobacterium-mediated transformation is provided in FIG. 1. The T-DNA from PHP36676 that was transferred to maize event DP 033121-3 is provided in FIG. 2. The cry2A.127, cry1A.88, vip3Aa20, and mo-pat probes were used in Southern blot hybridizations to evaluate the insertion in maize event DP-033121-3.

The restriction enzymes Sca I and Nco I were used to confirm the copy number of the PHP36676 T DNA insertions in maize event DP-033121-3. Sca I has five sites within the PHP36676 T-DNA, including one within the cry1A.88 gene at by 10,180. Nco I has four sites within the PHP36676 T-DNA, including one before the cry1A.88 gene at by 9,236. With Sca I digestion, a fragment of greater than 10,179 bp should hybridize to the probe for cry2A.127. With the Nco I digestion, a fragment of greater than 15,032 bp should hybridize to the cry1A.88, vip3Aa20, and mo pat probes. The absence of any other transgene-derived bands provides a strong indication that there is a single copy of each gene from the PHP36676 T-DNA in the maize genome.

The results of the Southern blot analysis with Sca I and Nco I and the cry2A.127, cry1A.88, vip3Aa20, and pat gene probes are provided in Table 6. Eight plants of the S1 generation of DP 033121-3 were analyzed, including two null segregant plants. The positive plants showed a single band of the expected size, thus indicating that a single copy of the T DNA was integrated into the genome of event DP-033121-3. A band of greater than 10,179 bp was observed with the cry2A.127 probe in the Sca I digest, which is consistent with the expected fragment size. A band of greater than 15,032 bp was observed with the cry1A.88 probe with the Nco I digest, which is consistent with the expected fragment size. A band of greater than 15,032 bp was observed with the vip3Aa20 probe with the Nco I digest, which is consistent with the expected fragment size. A band of greater than 15,032 bp was observed with the mo-pat probe with the Nco I digest, which is consistent with the expected fragment size. Additional bands due to hybridization of the mo-pat probe to maize genomic DNA sequences were observed in both control and transgenic samples. As expected based on the T-DNA map and Nco I digestion (FIG. 2), the cry1A.88, vip3Aa20, and mo pat probes appear to have all hybridized to the same size fragment for event DP-033121-3.

This Southern blot analysis indicates that the T-DNA in event DP-033121-3 derived from construct PHP36676 is inserted as a single copy.

TABLE 6

| | | Expected Fragment | Observed Fragment Size in |
|---|---|---|---|
| | Enzyme | Size from PHP36676 | DP-033121-3 Maize |
| Probe | Digest | T-DNA (bp)^{a} | (bp)^{b} |
| cry2A.127 | Sca I | >10,200 | >8,600 |
| cry1A.88 | Nco I | >15,000 | >8,600 |
| vip3Aa20 | Nco I | >15,000 | >8,600 |
| mo-pat | Nco I | >15,000 | >8,600 |

^{a}Expected fragment sizes based on map of PHP36676 T-DNA (FIG. 2). Expected sizes are rounded to the nearest 100 bp.
^{b}All observed fragment sizes are approximated based on the migration of the DIG VII molecular weight marker.

### Example 4

### Sequence Characterization of Insert and Genomic Flanking Regions of Maize Event DP-033121-3

Maize (Zea mays L.) event DP-033121-3 (033121 maize) was modified by the insertion of the T-DNA region from plasmid PHP36676 which contains four gene cassettes as disclosed above. Expression of the Vip3Aa20, Cry2A.127, and Cry1A.88 proteins confers resistance to certain lepidopteran insects.

Total genomic DNA was extracted from approximately 1 gram of frozen leaf tissue. The PHP36676 T-DNA insert/flanking genomic border regions were amplified by PCR. Each PCR fragment was then cloned into a commercially available plasmid vector and characterized by Sanger DNA sequencing. Individual sequence reads were assembled and manually inspected for accuracy and quality. A consensus sequence of the insert and 5′ and 3′ flanking sequence (SEQ ID NO: 14) of event DP-033121-3 was generated by majority-rule.

### Example 5

### Event-Specific Identification System Maize Event DP-033121-3

The event-specific PCR assay for DP-033121-3 maize was designed at the 5′ junction between the genomic DNA and the 33121 insert. The forward primer (12-O-4861 SEQ ID NO: 6) is situated within maize genomic DNA. The reverse primer (12-O-48628 SEQ ID NO: 7) is situated within the inserted DNA and the probe (12-Q-P219 SEQ ID NO: 8) spans the junction. Hereafter, this event-specific PCR assay for 33121 maize will be referred to as the 33121 assay.

A 15 μL aliquot of the thoroughly mixed master mixes are dispensed into each appropriate well of a reaction plate. A 5 μL aliquot of the Standards and 5 μL aliquots of the 40 ng/μL unknown samples are dispensed into the appropriate wells. For the NTCs, 5 μL of the diluent that was used for preparing the unknowns and standards (e.g. water or dilution buffer) is added to the appropriate wells instead of genomic DNA. Table 7 shows the 33121 assay primers and resulting amplicon (SEQ ID NO: 9). Table 8 shows the preparation of the 33121 assay master mix. Table 9 shows the PCR cycle profile for the 33121 assay. The resulting DP-033121-3 assay amplicon sequence (Length: 76 bp) is shown in SEQ ID NO: 9. The DP-033121-3 inserted DNA sequence is in bold; the primer and probe binding sites are underlined.

SEQ ID NO: 9
GCAAGAACCCGAAGAAACTCATTCTATTTAG
TATTGAGACAAACACTGATAGTT
TAAACTGAAGGCGGGAAACGAC

TABLE 7

| Name | Sequence (5′ to 3′) | SEQ ID NO: |
|---|---|---|
| 12-O-4861 | GCAAGAACCCGAAGAAACT | SEQ ID NO: 6 |
| (forward primer) | CATT | |
| 12-O-4862 | GTCGTTTCCCGCCTTCAGT | SEQ ID NO: 7 |
| (reverse primer) | | |
| 12-Q-P219 | TATTGAGACAAACACTGAT | SEQ ID NO: 8 |
| (probe) | AGTT | |

TABLE 8

| | Stock | Final | |
|---|---|---|---|
| Component | Concentration | Concentraion | μL/rxn |
| TaqMan ® Universal PCR Master Mix, | 2 x | 1 x | 10.0 |
| No AmpErase ® UNG | | | |
| 12-O-4861 (forward primer) | 10 μM | 750 nM | 1.5 |
| SEQ ID NO: 6 | | | |
| 12-O-4862 (reverse primer) | 10 μM | 750 nM | 1.5 |
| SEQ ID NO: 7 | | | |
| 12-QP219 (probe) | 10 μM | 200 nM | 0.4 |
| SEQ ID NO: 8 | | | |
| Molecular grade water | | | 1.6 |
| Total volume* | | | 15.0 |

*Total PCR reaction volume is 20 μL (15 μL master mix and 5 μL genomic DNA template)

TABLE 9

| | | | Temperature | Time | Data | # of |
|---|---|---|---|---|---|---|
| Step | Cycle Element | | (° C.) | (min:sec) | Collection | Cycles |
| 1 | Initial enzyme activation | | 95 | 10:00 | no | 1x |
| 2 | Amplification | Denaturation | 95 | 0:15 | no | 40x |
| 3 | | | | | | |

The maize-specific reference PCR assay used for relative quantification is a pre-validated maize-specific PCR assay (EU-RL-GMFF, 2005) for Zea mays L. High Mobility Group (HMG) Protein A gene (hmgA) (Krech et al., Gene 234: 45-501999). Hereafter this maize-specific reference assay will be referred to as the HMG assay. The HMG assay amplifies a 79 bp product based upon GenBank Accession No. AJ131373. Table 10 shows the HMG assay primers and resulting amplicon (SEQ ID NO: 13). Table 11 shows the preparation of the HGM assay master mix. Table 9 shows the PCR cycle profile for the HGM assay.

TALBE 10

| Name | Sequence (5′ to 3′) | SEQ ID NO: |
|---|---|---|
| MaiJ-F2 | TTGGACTAGAAATCTCGTG | SEQ ID NO: 10 |
| (forward primer) | CTGA | |
| mhmg-rev | GCTACATAGGGAGCCTTGT | SEQ ID NO: 11 |
| (reverse primer) | CCT | |
| mhmg-probe | CAATCCACACAAACGCACG | SEQ ID NO: 12 |
| (probe) | CGTA | |

The resulting HMG assay amplicon sequence (Length: 79 bp) is shown in SEQ ID NO: 13. The primer and probe binding sites are underlined.

SEQ ID NO: 13
TTGGACTAGAAATCTCGTGCTGATTAATTGTTTTACGCGTGCGTTTGTG
TGGATTGTAGGACAAGGCTCCCTATGTAGC

TABLE 11

| | Stock | Final | |
|---|---|---|---|
| Component | Concentration | Concentration | μL/rxn |
| TaqMan ® Universal PCR Master | 2 x | 1 x | 10.0 |
| Mix, No AmpErase ® UNG | | | |
| MaiJ-F2 (forward primer) | 10 μM | 400 nM | 0.8 |
| SEQ ID NO: 10 | | | |
| mhmg-rev (reverse primer) | 10 μM | 400 nM | 0.8 |
| SEQ ID NO: 11 | | | |
| mhmg-probe (probe) | 10 μM | 150 nM | 0.3 |
| SEQ ID NO: 12 | | | |
| Molecular grade water | | | 3.1 |
| Total volume* | | | 15.0 |

*Total PCR reaction volume is 20 μL (15 μL master mix and 5 μL genomic DNA template)

The real-time PCR method has been optimized and validated using an Applied Biosystems ViiA™ 7 system. The PCR product is measured during each cycle (real-time) by means of a target-specific oligonucleotide probe labeled with two fluorescent dyes: FAM as a reporter dye at its 5′ end and either a non-fluorescent quencher (MGB for 12-Q219 in the event-specific 33121 maize assay) or a fluorescent quencher (TAMRA for HMG probe in the maize-specific reference assay) at its 3′ end. The 5′ nuclease activity of Taq DNA polymerase cleaves the probe and liberates the fluorescent moiety during the amplification process. The resulting increase in fluorescence during amplification is measured and recorded. The recommended method format makes use of 200 ng of template DNA per reaction. This corresponds to approximately 73,394 haploid copies of the Zea mays genome, assuming a genome weight of 2.725 pg (Arumuganathan and Earle, 1991). The unknown samples are diluted to 40 ng/μL in water or dilution buffer. A 5 μL aliquot of each unknown sample is used in triplicate for both the HMG and 33121 assays.

The method format uses the standard curves for the two PCR assays (the 33121 assay and the HMG assay) comprised of four standard points, each measured in triplicate. The standards were produced by preparing a solution of 40 ng/μL of total genomic maize DNA with 10% 33121 maize (GM %) DNA followed by serial dilutions in dilution buffer (0.1×TE buffer+10 ng/μL salmon sperm DNA). The no-template controls (hereafter referred to as NTCs) were run in triplicate in each assay as negative controls to verify purity of reagents. Each sample (unknown) is analyzed using 200 ng genomic maize DNA per reaction. Analysis was performed in triplicate (6 reactions per sample in total for both PCR assays). The relative content of 33121 maize to total maize DNA was subsequently calculated by determining the mean of the copy numbers based on the standard curves (linear regression of C_{T} value versus log [copy number]) and calculating the ratios of 33121 maize copy number to total copy number of haploid maize genomes.

This event-specific quantitative PCR system for detection of DP-033121-3 maize DNA was developed, optimized, and validated on Applied Biosystems' ViiA 7™ real-time PCR system. The method can also be applied on a different platform however, with minimal optimization and adaptation.

The event-specific real-time PCR method described here can be applied to determine the relative content of DP-033121-3 maize DNA in total genomic maize DNA. The method performs in a linear manner with an acceptable level of accuracy and precision over the whole range from 0.08% to 5.0% DP-033121-3 content. The method was developed and validated with genomic DNA extracted from maize seeds. However, the assay can be applied to any matrix from which genomic DNA with sufficient quantity and quality can be purified.

### Example 6

### Copy Number Determination by PCR of Maize Event DP-033121-3

Two generations of maize containing event DP-033121-3 were grown in cell-divided flats under typical greenhouse production conditions. Approximately 100 plants were grown for each generation. Leaf samples were collected from each plant twelve days after planting, when plants were at approximately the V2-V3 growth stage (i.e. when the collar of the second leaf becomes visible). Two leaf punches per plant were analyzed for the copy number of the PHP36676 T-DNA through copy number PCR for the co/1A.88, cry2A.127, vip3Aa20, and mo-pat genes.

For detection of the co/1A.88, cry2A.127, vip3Aa20, and mo-pat amplicons, between 85 and 120-bp of the region of each gene were amplified using primers specific for each unique sequence. Additionally, a TaqMan® probe and primer set for an endogenous reference gene was used for qualitative assessment of the assay and to demonstrate sufficient quality and quantity of DNA for PCR amplification. Each extracted DNA sample was analyzed in triplicate. The real-time PCR reaction exploits the 5′ nuclease activity of the hot-start DNA polymerase. Two primers and one probe anneal to the target DNA with the probe, which contains a 5′ fluorescent reporter dye and a 3′ quencher dye, sitting between the two primers. With each PCR cycle, the reporter dye is cleaved from the annealed probe by the polymerase, emitting a fluorescent signal that intensifies in each subsequent cycle. The cycle at which the emission intensity of the sample rises above the detection threshold is referred to as the C_{T} value. When no amplification occurs, there is no C_{T} calculated by the instrument and is equivalent to a C_{T} value of 40.00.

In order to determine the copy number of the test samples, single-copy calibrators (samples known to contain a single copy of the gene of interest) were used as controls for both the endogenous gene and gene of interest. The dC_{T} was calculated for the test samples and single-copy calibrators as described above. The ddC_{T} was then used to statistically calculate copy number (ddC_{T}=Single-copy calibrator dC_{T}−GOI dC_{T}). The algorithm tolerances were used to apply a copy number for each sample. A copy number of 1 was applied to the population producing a similar mean dCt when compared to the single copy calibrators. A copy number of 2 was applied if samples produced a ddC_{T} of 1.0 when compared to the single copy calibrators; and a copy number of 3 was applied if samples produced a ddC_{T} of 0.5 when compared to the 2-copy population. The statistical algorithm also applies probabilities of each potential copy number assignment based on the assigned ddC_{T} values following the analysis. Any ddC_{T} values falling outside expected ranges will produce copy number results with weak probabilities where ddC_{T} values within expected ranges will produce results with strong probabilities.

DNA was extracted from each sample using an alkaline buffer with high heat. Approximately 3 ng of template DNA was used per reaction. Reaction mixes were prepared, each comprised of all components to support both the gene of interest and the endogenous gene for the PCR reaction, except for DNA template. The endogenous reference assay was multiplexed with event DP-Ø33121-3 in the same PCR run. The extracted DNA was assayed using the appropriate primer and probe set in Applied Biosystems® Fast Advanced Master Mix with 30% Bovine Serum Albumin (BSA). Controls (no template controls; NTC) included water and TE buffer (10 mM Tris pH 8.0, 1 mM EDTA). Individual volumes of primer varied per reaction between 300 μM and 900 μM, dependent on the optimal concentration established during analysis validation. Annealing temperatures and number of cycles used during the PCR analysis are provided in Table 12. The primer and probes used for the PCR analysis are provided in Table 13.

TABLE 12

| | | | Temperature | Time | |
|---|---|---|---|---|---|
| Step | Description | | (° C.) | (seconds) | Cycles |
| 1 | Initial Denaturation | | 95 | 20 | 1 |
| 2a | Amplification | Denaturation | 95 | 1 | 40 |
| 2b | | Anneal/Extend | 60 | 20 | |

TABLE 13

| Reagent | Sequence (5′ to 3′) | |
|---|---|---|
| cry1A.88 | TCGAGAGATTGGATTCGGT | SEQ ID NO: 21 |
| forward primer | ACA | |
| cry1A.88 | GGGAACAGCGACACGATGT | SEQ ID NO: 22 |
| reverse primer | | |
| cry1A.88 probe | CGAGCTGACCCTCAC | SEQ ID NO: 23 |
| cry2A.127 | CGCACTTTCATCAGCGAGA | SEQ ID NO: 24 |
| forward primer | AG | |
| cry2A.127 | TGTTCTGCTCAAACCTCAG | SEQ ID NO: 25 |
| reverse primer | AGAAT | |
| cry2A.127 probe | TCGGCAACCAAGGC | SEQ ID NO: 26 |
| vip3Aa20 | ACCAGAGCGAGCAAATCTA | SEQ ID NO: 27 |
| forward primer | CTACA | |
| vip3Aa20 | TAGCGCAGGGTCTTCATCT | SEQ ID NO: 28 |
| reverse primer | TC | |
| vip3Aa20 probe | CGTGTTCCCGAACGAGTA | SEQ ID NO: 29 |
| mo-pat | CATCGTGAACCACTACATC | SEQ ID NO: 30 |
| forward primer | GAGAC | |
| mo-pat | GTCGATCCACTCCTGCGG | SEQ ID NO: 31 |
| reverse primer | | |
| mo-pat probe | ACCGTGAACTTCCGCACCG | SEQ ID NO: 32 |
| | AGC | |

Results are provided in Table 14. The results of the qPCR copy number analysis indicate stable integration and segregation of a single copy of the transgenes with transfer to subsequent generations.

TABLE 14

| | | | | Avg | Avg | Copy |
|---|---|---|---|---|---|---|
| Event | Generation | #Plants | Transgene | C_{T}† | dC_{T}‡ | Number |
| DP- | BC1F1*¹ | 15 | cry1A.88 | 28.54 | −0.60 | 1 |
| Ø33121-3 | | | cry2A.127 | 28.72 | −1.09 | 1 |
| | | | vip3Aα20 | 29.69 | −1.92 | 1 |
| | | | mo-pat | 29.76 | −1.02 | 1 |
| | BC2F1*¹ | 15 | cry1A.88 | 28.55 | −0.58 | 1 |
| | | | cry2A.127 | 28.80 | −1.07 | 1 |
| | | | vip3Aα20 | 29.77 | −1.90 | 1 |
| | | | mo-pat | 29.41 | −0.95 | 1 |

†An Average C_{T} of 40 is a value automatically assigned by the scoring software tool used to determine copy number estimation where the Real-Time PCR instrument algorithm does not assign a C_{T} value. This assignment is to manage raw data import into the database and to allow a calculation of a dC_{T}.
‡dC_{T} is equivalent to C_{T} Endogenous − C_{T}t Gene of Interest. The average value is comprised of the values supporting each represented plant for the copy number group, analyzed in triplicate.

### Example 7

### Protein Expression and Concentration

Maize lines containing event DP-Ø33121-3 were grown in 4-inch pots, organized in flats containing 15 pots, using typical greenhouse production conditions in 2013 in Johnston, Iowa, USA. Approximately 15 plants from segregating populations were transplanted to 2-gallon (7.6 L) pots and grown for each of the following generations of 33121 maize. Each plant tested positive for event DP-033121-3 via PCR analysis. Leaf samples were collected from each plant at approximately the V9 growth stage (i.e. when the collar of the ninth leaf becomes visible). One leaf per plant was obtained by selecting the youngest leaf that had emerged at least 8 inches (20 cm) from the whorl. The leaf was pruned (cut) from the plant approximately 8 inches (20 cm) from the leaf tip. The leaf sample (including midrib) was cut into ≦1 inch (2.5 cm) pieces and placed in a 50-ml sample vial. The samples were then placed on dry ice until transferred to a freezer (≦−10° C.). All leaf samples were lyophilized, under vacuum, until dry and then finely homogenized in preparation for expressed trait protein analysis. Samples were stored frozen between processing steps.

Concentrations of the Cry1A.88, Cry2A.127, Vip3Aa20, and PAT proteins were determined using specific quantitative ELISA methods. Aliquots of processed leaf tissue samples were weighed into 1.2-ml tubes at the target weight of 10 mg. Each sample analyzed for Cry1A.88 protein concentrations was extracted in 0.6 ml of chilled PBST (phosphate buffered saline with 0.05% Tween-20®) and 4M urea. Each sample analyzed for Cry2A.127, Vip3Aa20, and PAT protein concentrations was extracted in 0.6 ml of chilled PBST. Following centrifugation, supernatants were removed, diluted in PBST, and analyzed. Standards (typically analyzed in triplicate wells) and diluted samples (typically analyzed in duplicate wells) were incubated in a plate pre-coated with a Cry1A.88, Cry2A.127, Vip3Aa20, or PAT antibody. Following incubation, unbound substances were washed from the plate. A different Cry1A.88, Cry2A.127, Vip3Aa20, and PAT antibody, conjugated to the enzyme horseradish peroxidase (HRP), was added to the plate and incubated. Unbound substances were washed from the plate. Detection of the bound Cry1A.88-antibody complex was accomplished by the addition of substrate, which generated a colored product in the presence of HRP. The reaction was stopped with an acid solution and the optical density (OD) of each well was determined using a plate reader.

### Calculations for Determining Protein Concentrations

SoftMax® Pro GxP (Molecular Devices Corporation Sunnyvale, Calif., USA) software was used to perform the calculations required to convert the OD values obtained for each set of duplicate sample wells to a protein concentration value. A standard curve was included on each ELISA plate. The equation for the standard curve was generated by the software, which used a quadratic fit to relate the OD values obtained for each set of triplicate standard wells to the respective standard concentration (ng/ml).

The quadratic regression equation was applied as follows:

y=Cx²+Bx+A

Where x=known standard concentration and y=respective mean absorbance value (OD)

### Sample Concentration

Interpolation of the sample concentration (ng/ml) was accomplished by solving for x in the above equation using values for A, B, and C determined by the standard curve.

$$Sample Concentration ( ng \text{/} ml ) = \frac{- B + \sqrt{{B}^{2} - 4 C ( A - sample OD )}}{2 C}$$

e.g. Curve Parameters: A=0.0476, B=0.4556, C=−0.01910, and sample OD=1.438

$$Sample Concentration = \frac{- 0.4556 + \sqrt{{0.4556}^{2} - 4 ( - 0.01910 ) ( 0.0476 - 1.438 )}}{2 ( - 0.01910 )} = 3.6 ng \text{/} ml$$

Sample concentration values were adjusted for the dilution factor expressed as 1:N

### Adjusted Concentration=Sample Concentration×Dilution Factor

e.g. Sample Concentration=3.6 ng/ml and Dilution Factor=1:10

Adjusted Concentration=3.6 ng/ml×10=36 ng/ml

Adjusted sample concentration values were converted from ng/ml to ng/mg sample weight as follows:
ng/mg Sample Weight=ng/ml×Extraction Volume (ml)/Sample Weight (mg)
   e.g. Concentration=36 ng/ml, Extraction Volume=0.60 ml, and Sample Weight=10.0 mg
ng/mg Sample Weight=36 ng/mg×0.60 ml/10.0 mg=2.2 ng/mg

### Lower Limit of Quantification (LLOQ)

The LLOQ, in ng/mg sample weight, was calculated as follows:

$$OQ = \frac{Reportable Assay LLOQ \times Extraction Volume}{Sample Target Weight}$$

e.g. for PAT in leaf: reportable assay LLOQ=2.3 ng/ml, extraction volume=0.6 ml, and sample target weight=10 mg

$$LLOQ = \frac{2.3 ng \text{/} ml \times 0.6 ml}{10 g} = 0.14 ng \text{/} mg sample weight$$

The proteins Cry1A.88, Cry2A.127, Vip3Aa20, and PAT were detected in V9 leaf tissue from two generations of 33121 maize. Results are shown in Table 15.

TABLE 15

| | | # of | | Protein Concentration in ng/mg Dry Weight | | | |
|---|---|---|---|---|---|---|---|
| Event | Gen. | Samples | | Cry1A.88 | Cry2A.127 | Vip3Aa20 | PAT |
| DP- | BC1F1*¹ | 15 | Mean ± SD | 12 ± 2.6 | 95 ± 16 | 36 ± 8.8 | 15 ± 2.5 |
| Ø33121-3 | | | Range | 6.6-15 | 52-120 | 25-52 | 12-21 |
| | BC2F1*¹ | 15 | Mean ± SD | 12 ± 1.1 | 110 ± 21 | 43 ± 10 | 17 ± 2.4 |
| | | | Range | 11-14 | 78-150 | 21-60 | 13-22 |

### Example 8

### Insect Efficacy of Maize Event DP-033121-3

### European Corn Borer Efficacy

Efficacy field testing was conducted against ECB maize in F1 generation DP-Ø33121-3 and a near-isoline control maize (the near-isoline control maize had the same background as DP-Ø33121-3 maize). Single-row plots (5 plants/row) were planted in a randomized complete block with two replications. All plants were sampled to confirm the presence of the traits by PCR. ECB data was evaluated by stalk tunneling and was measured approximately 48 to 56 days, depending on location, after the last successful ECB infestation. The stalks of all infested plants from were split in half longitudinally (using a knife) from the top of the 4^{th} internode above the primary ear to the base of the plant. The total length of ECB stalk tunneling (ECBXCM) was then measured in centimeters and recorded for each plant.

### Fall Armyworm Efficacy

Efficacy field testing was conducted against FAW in F1 generation DP-Ø33121-3 maize and a near-isoline control maize (the near-isoline control maize had the same background as DP-Ø33121-3 maize). Single-row plots (5 plants/row) were planted in a randomized complete block with two replications. All plants were sampled to confirm the presence of the traits by PCR. Injury from FAW foliar feeding was scored approximately three weeks after infestation. Injury from FAW feeding was recorded using a 9 to 1 visual rating scale (FAWLF) where a score of “9” indicated “no damage” and a score of “1” indicated “heavy damage” (Table 16). The visual rating scale is similar to that published by Davis et al. (1992 Mississippi Agric. and Forestry Exp. Stat. Tech Bull. 186), with the numbering in reverse order.

TABLE 16

| FAWLF | |
|---|---|
| Score^{a} | Observations |
| 9 | No damage to pinhole lesions present on whorl leaves. |
| 8 | Pinholes and small circular lesions present on whorl leaves. |
| 7 | Small circular lesions and a few small elongated (rectangular shaped) lesions |
| | of up to 1.3 cm (½″) in length present on whorl and furl leaves. |
| 6 | Several small to size 1.3 to 2.5 cm (½″ to 1″) in length elongated lesions |
| | present on a few whorl and furl leaves. |
| 5 | Several large elongated lesions greater than 2.5 cm (1″) in length present on |
| | a few whorl and furl leaves and/or a few small to mid-sized uniform to |
| | irregular shaped holes (basement membrane consumed) eaten from the whorl |
| | and or furl leaves. |
| 4 | Several large elongated lesions present on several whorl and furl leaves |
| | and/or several large uniform to irregular shaped holes eaten from the whorl |
| | and furl leaves. |
| 3 | Many elongated lesions of all sizes present on several whorl leaves plus |
| | several large uniform to irregular shaped holes eaten from the whorl and furl |
| | leaves. |
| 2 | Many elongated lesions of all sizes present on most whorl and furl leaves |
| | plus many mid to large-sized uniform to irregular shaped holes eaten from |
| | the whorl and furl leaves. |
| 1 | Whorl and furl leaves almost totally destroyed |

^{a}Adapted from Davis, et al. 1995

### Corn Earworm Efficacy

Efficacy field testing was conducted in against CEW in the F1 generation DP-Ø33121-3 maize and a near-isoline control maize (the near-isoline control maize had the same background as DP-Ø33121-3 maize). Single-row plots (5 plants/row) were planted in a randomized complete block with three replications. All plants were sampled to confirm the presence of the traits by PCR. The natural infestation was supplemented with manually-infested neonate CEW when plants reached approximately growth stage R1. Neonates were infested with a hand-held applicator that dispensed larvae dispersed with corn cob grits onto the silks of the primary ear on each plant. The applicators were calibrated to deliver approximately 56 neonates per shot and 1 shot was applied to each plant. Injury from CEW ear feeding was scored 26 days after infesting. Injury from CEW feeding was assessed by measuring the total square centimeters of kernel damage to the primary ears. Damage to the cob tip where no kernels had formed was not included in the measurement. The total CEW square centimeters of ear damage (CEWSCM) was recorded for each plant.

### Herbicide Efficacy (Glufosinate)

The PAT protein expressed in plants confers tolerance to herbicides containing glufosinate. In order to confirm efficacy, a bioassay was performed on DP-Ø33121-3 maize from the BC2F1 segregating generation. Seeds from DP-Ø33121-3 maize were planted in cell-divided flats under typical greenhouse production. Approximately 100 seed from the segregating population were planted for DP-Ø33121-3 maize. The presence or absence of the traits was confirmed through PCR. The plants were assigned to two groups based on the PCR scores: 1) positive for event and 2) negative, null isoline control. Thirteen days after planting, a herbicide spray mixture was applied to all plants containing Ignite 280 SL®′ which contains 24.5% glufosinate-ammonium, equivalent to 2.3 pounds active ingredient (ai) per gallon (280 grams ai per liter). Ammonium sulfate was added to the spray mixture at a rate of 3.0 pounds per acre (3.4 kilograms per hectare). No other adjuvants or additives were included in the spray mixture. The spray mixture was applied at a rate of 21 gallons per acre (196 liters per hectare), equivalent to 0.40 pounds glufosinate ai per acre (0.45 kilograms ai per hectare) using a spray chamber to simulate a broadcast (over-the-top) application. All plants were evaluated approximately 7 days after herbicide application. Tolerance was visually evaluated by herbicide injury: plants with no herbicide injury/healthy plant were designated as “tolerant” and plants with herbicide injury or death were designated as “not tolerant.” Table 17 shows the results from the ECB, FAW, CEW, and glufosinate efficacy analyses.

TABLE 17

| | Mean ± Standard Deviation | |
|---|---|---|
| | (Min-Max) | |
| Efficacy Analysis | DP-Ø33121-3 Maize | Negative Control Maize |
| European Corn Borer | 0.5 ± 1.1 | 21.7 ± 9.1 |
| (Lepidoptera)^{a} | (0-3) | (8-45) |
| Fall Armyworm | 9 ± 0 | 5 ± 1.4 |
| (Lepidoptera)^{b} | (NA^{e}) | (3-9) |
| Corn Earworm | 2.1 ± 3.2 | 24.4 ± 16.1 |
| (Lepidoptera)^{c} | (0-13) | (0.8-73) |
| Glufosinate^{d} | Tolerant | Not Tolerant |

^{a}n = 10 for DP-Ø33121-3 maize and n = 20 for the near-isoline control maize.
^{b}n = 15 for DP-Ø33121-3 maize and n = 30 for the near-isoline control maize.
^{c}n= 15 for DP-Ø33121-3 maize and n = 30 for the near-isoline control maize.
^{d}n = 49 for DP-Ø33121-3 maize; and n = 109 for negative control maize.
^{e}NA: Due to lack of variability in the data, no min/max could be calculated.

Having illustrated and described the principles of the present disclosure, it should be apparent to persons skilled in the art that the disclosure can be modified in arrangement and detail without departing from such principles. We claim all modifications that are within the spirit and scope of the appended claims.

All publications and published patent documents cited in this specification are incorporated herein by reference to the same extent as if each individual publication or patent application was specifically and individually indicated to be incorporated by reference.

What is claimed is:

1. A DNA construct comprising:
(a) a first expression cassette, comprising in operable linkage:
(i) a full length Citrus Yellow Mosaic virus (CYMV) promoter;
(ii) a maize adh1 first intron;
(iii) a synthetic chloroplast targeting peptide
(iv) a Cry2A.127 encoding DNA molecule;
(v) a ubiquitin3 (UBQ3) transcriptional terminator; and
(vi) a 3′ untranslated region of an Arabidopsis ribosomal protein gene;
(b) a second expression cassette, comprising in operable linkage:
(i) a truncated BSV promoter and second adh1 intron;
(ii) a Cry1A.88 encoding DNA molecule; and
(iii) a sorghum actin transcriptional terminator;
(c) a third expression cassette, comprising in operable linkage:
(i) a maize polyubiquitin promoter;
(ii) a 5′ untranslated region and intron1 of a maize polyubiquitin gene;
(iii) a Vip3Aa20 encoding DNA molecule; and
(iv) a pinII transcriptional terminator; and
(d) a fourth expression cassette comprising in operable linkage:
(i) a maize polyubiquitin promoter;
(ii) a mo-pat encoding DNA molecule; and
(ii) a pinII transcriptional terminator.

2. The DNA construct of claim 1, comprising the sequence of SEQ ID NO: 1.

3. The DNA construct of claim 1, wherein the DNA construct is flanked by the 5′ junction sequence of SEQ ID NO: 15 and the 3′ junction sequence of SEQ ID NO: 16.

4. A plant transformed with the DNA construct of claim 1, 2 or 3.

5. A corn plant, comprising the sequence of SEQ ID NO: 14 that exhibits resistance to one or more lepidopteran pests.

6. A corn event DP-033121-3, wherein a representative sample of seed of said corn event has been deposited with American Type Culture Collection (ATCC) with Accession No. PTA-13392.

7. Plant parts of the corn event DP-033121-3 of claim 6.

8. Seed of corn event DP-033121-3, wherein said seed comprises a DNA molecule of SEQ ID NO: 14.

9. A corn plant, or part thereof, grown from the seed of claim 8.

10. A transgenic seed produced from the corn plant of claim 8, comprising event DP-033121-3.

11. A transgenic corn plant, or part thereof, grown from the seed of claim 9.

12. An isolated nucleic acid molecule comprising a nucleotide sequence selected from the group consisting of SEQ ID NO: 9; SEQ ID NO: 14; SEQ ID NO: 8, and full length complements thereof.

13. An amplicon comprising the nucleic acid sequence selected from the group consisting of SEQ ID NO: 9, and full length complements thereof.

14. A biological sample derived from corn event DP-033121-2 plant, tissue, or seed, wherein said sample comprises a nucleotide sequence selected from the group consisting of SEQ ID NO: 9, SEQ ID NO: 14, SEQ ID NO: 8 and the complement thereof, wherein said nucleotide sequence is detectable in said sample using a nucleic acid amplification or nucleic acid hybridization method, wherein a representative sample of said corn event DP-033121-3 seed of has been deposited with American Type Culture Collection (ATCC) with Accession No. PTA-13392.

15. The biological sample of claim 13, wherein said biological sample comprises plant, tissue, or seed of transgenic corn event DP-033121-3.

16. The biological sample of claim 14, wherein said biological sample is a DNA sample extracted from the transgenic corn plant event DP-033121-3, and wherein said DNA sample comprises one or more of the nucleotide sequences selected from the group consisting of SEQ ID NO: 9, SEQ ID NO: 14, SEQ ID NO: 8, and the complement thereof.

17. The biological sample of claim 15, wherein said biological sample is selected from the group consisting of corn flour, corn meal, corn syrup, and cereals manufactured in whole or in part to contain corn by-products.

18. A method for producing a corn plant resistant to lepidopteran pests, comprising:
(a) sexually crossing a first parent corn plant with a second parent corn plant, wherein said first or second parent corn plant comprises event DP-033121-3 DNA, thereby producing a plurality of first generation progeny plants;
(b) selecting a first generation progeny plant that is resistant to lepidopteran insect infestation;
(c) selfing the first generation progeny plant, thereby producing a plurality of second generation progeny plants; and
(d) selecting from the second generation progeny plants, a plant that is resistant to lepidopteran pests;
wherein the second generation progeny plants comprise event DP-033121-3 DNA.

19. A method of producing hybrid corn seeds comprising:
(a) planting seeds of a first inbred corn line comprising the DNA construct of claim 1 and seeds of a second inbred line having a genotype different from the first inbred corn line;
(b) cultivating corn plants resulting from said planting until time of flowering;
(c) emasculating said flowers of plants of one of the corn inbred lines;
(d) sexually crossing the two different inbred lines with each other; and
(e) harvesting the hybrid seed produced thereby.

20. The method of claim 19 further comprising the step of backcrossing the second generation progeny plant of step (d) that comprises corn event DP-033121-3 DNA to the parent plant that lacks the corn event DP-033121-3 DNA, thereby producing a backcross progeny plant that is resistant to at least lepidopteran insects.

21. A method for producing a corn plant resistant to at least lepidopteran insects, said method comprising:
(a) sexually crossing a first parent corn plant with a second parent corn plant, wherein said first or second parent corn plant is a corn event DP-033121-3 plant, thereby producing a plurality of first generation progeny plants;
(b) selecting a first generation progeny plant that is resistant to at least lepidopteran insect infestation;
(c) backcrossing the first generation progeny plant of step (b) with the parent plant that lacks corn event DP-033121-3 DNA, thereby producing a plurality of backcross progeny plants; and
(d) selecting from the backcross progeny plants, a plant that is resistant to at least lepidopteran insect infestation;
wherein the selected backcross progeny plant of step (d) comprises SEQ ID NO:14.

22. The method according to claim 21, wherein the plants of the first inbred corn line are the female parents or male parents.

23. Hybrid seed produced by the method of claim 21.

24. A method of detecting the presence of a nucleic acid molecule that is unique to event DP-033121-3 in a sample comprising corn nucleic acids, the method comprising:
(a) contacting the sample with a pair of primers that, when used in a nucleic-acid amplification reaction with genomic DNA from event DP-033121-3 produces an amplicon that is diagnostic for event DP-033121-3;
(b) performing a nucleic acid amplification reaction, thereby producing the amplicon; and
(c) detecting the amplicon.

25. A pair of polynucleotide primers comprising a first polynucleotide primer and a second polynucleotide primer which function together in the presence of event DP-033121-3 DNA template in a sample to produce an amplicon diagnostic for event DP-033121-3.

26. The pair of polynucleotide primers according to claim 25, wherein the sequence of the first polynucleotide primer is or is complementary to a corn plant genome sequence flanking the point of insertion of a heterologous DNA sequence inserted into the corn plant genome of event DP-033121-3, and the sequence of the second polynucleotide primer is or is complementary to the heterologous DNA sequence inserted into the genome of event DP-033121-3.

27. A method of detecting the presence of DNA corresponding to the DP-033121-3 event in a sample, the method comprising:
(a) contacting the sample comprising maize DNA with a polynucleotide probe that hybridizes under stringent hybridization conditions with DNA from maize event DP-033121-3 and does not hybridize under said stringent hybridization conditions with a non-DP-033121-3 maize plant DNA;
(b) subjecting the sample and probe to stringent hybridization conditions; and
(c) detecting hybridization of the probe to the DNA;
wherein detection of hybridization indicates the presence of the DP-033121-3 event.

28. A kit for detecting nucleic acids that are unique to event DP-033121-3 comprising at least one nucleic acid molecule of sufficient length of contiguous polynucleotides to function as a primer or probe in a nucleic acid detection method, and which upon amplification of or hybridization to a target nucleic acid sequence in a sample followed by detection of the amplicon or hybridization to the target sequence, are diagnostic for the presence of nucleic acid sequences unique to event DP-033121-3 in the sample.

29. The kit according to claim 28, wherein the nucleic acid molecule comprises a nucleotide sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12 and SEQ ID NO: 13.

30. A kit for detecting in a plant or plant part an insecticidal protein of event DP-033121-3, wherein the kit comprises at least one antibody specific Cry2A.127, Cry1A.88 or Vip3Aa20.


SEQUENCE LISTING (32 sequences)

SEQ ID NO: 1 - DNA, 24266 bp, Artificial Sequence
  PHP36676 T-DNA
gtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcggttactaccggctggatggcggggccttgatcgtgcaccgccggcgtccggactaactaactagtcgagctagttaccctatgaggtgacatgaagcgctcacggttactatgacggttagcttcacgactgttggtggcagtagcgtacgacttagctatagttccggacttacccttaagataacttcgtatagcatacattatacgaagttatgggcccaccggtggtaccgagctcgtttaaacgctcttcaactggaagagcggttaccagagctggtcacctttgtccaccaagatggaactggcgcgcctcattaattaagtcagcggccgctctagttgaagacacgttcatgtcttcatcgtaagaagacactcagtagtcttcggccagaatggccatctggattcagcaggcctagaaggccatttatctatcaactttgtataataaagttgcccggtccttaggcggaccgggccatctaggccgcggccgcactgtcaagctattattagcttctttaataagtccaatgtgaacaaaccgtctagggttagatggattgctttcacagatttccttactggtctaggaatccctgtaaatatagagcacatagatggaaaaaataaccatctggctgatgctctgtccagattagtaactggttttgtttttgcagaaccacaatgtcaagacaagttccaggacgatttagggaaattggaagcagctcttcaggagaagaaagaggctccgcaagcaatgcacgtagaatatgtctccctgttgatcagatcagcggaccgcattacccgctcgctctgctttatgagggactcgtctcacagcagaatttactcatgcaggccaggcaaagaaccaatgaaggccttaatctgcgaacagaagtcatgccaatccaaaggcgacttagggaatacgaggactgtgcactccaagagtgcattcaatcagcaagacaactggtggccctccaccagcacaaactcgcttacatcagaagcaaagctacaagggacaacgcatatgccgataggctacccacatgcaatcgggaccacgagcaactgtgtgaagtggtcgagctattagaaggaatctcggaaagaatcagcgatacagctgtctaggacagctggcttcaattatggagcgtgatggacccccccgcaataatccaaagtttggtgtgcttttagtagtgcgtctttatggaccactactttattgtaataatcgatgctttttgtagtgcgctcttcgtgcgctctactttatgcttttgcttttgtaagtgcgctgtaagtgcgcctgtctttcttcagatgcttatcctttaagcatcttttgctttttgcgtggcatcctttagttcacaatttaaagaatgacgatggggcccaagatgtgcacccggttctctaaattgcctatataaggatatgccatagccttgtttttgcaagtcaggaatacctgagcataacttggctaagcaaaagtttgtaagtgttctaagctttcatttgtaaactttctgtttggttttaataaaatctctcgtcaatcgttgtgaacatatattgtttgtttgtattgttgtatcttatttgttgtggtgataaggatcttcgatatcccggactggcgccaggtccgccttgtttctcctctgtctcttgatctgactaatcttggtttatgattcgttgagtaattttggggaaagcttcgtccacagtttttttttcgatgaacagtgccgcagtggcgctgatcttgtatgctatcctgcaatcgtggtgaacttatttcttttatatccttcactcccatgaaaaggctagtaatctttctcgatgtaacatcgtccagcactgctattaccgtgtggtccatccgacagtctggctgaacacatcatacgatattgagcaaagatcgatctatcttccctgttctttaatgaaagacgtcattttcatcagtatgatctaagaatgttgcaacttgcaaggaggcgtttctttctttgaatttaactaactcgttgagtggccctgtttctcggacgtaaggcctttgctgctccacacatgtccattcgaattttaccgtgtttagcaagggcgaaaagtttgcatcttgatgatttagcttgactatgcgattgctttcctggacccgtgcagctggcgccttgggatccatggctgcgaccactctcacgagcgctctcccaggagcctttagcagctctcagagaccttcggctccgttcaacctccagaggagccctagagtcctcagacgcttcaaccgcaagaccggtagacagccacgcggtctcgtcagagctgctaaggctcagcgctctggtaccagatccatgggcaactccgttctcaattccggaaggactacgatctgtgatgcgtacaacgttgcagctcatgatccgttctcattccagcacaagtcacttgacactgttcagagggagtggactgagtggaagaagaacaaccattcgctgtatctcgatccgatcgttggaactgtggcttcattcctgctcaagaaggtcggttctctcgttggtaagaggattctctcggaactcaggaacttgatcttcccatctggtagcacaaacctcatgcaggacatacttagggaaactgagcagttcctgaaccaacgccttgacactgataccttggcaagggtcaatgctgagttgacaggtcttcaagcgaacgttgaggagttcaatcgccaagttgacaacttccttaaccctaaccggaatgccgttcctctgtctatcacgtcatctgtcaacacgatgcagcagctgttcttgaaccggcttcctcaattccagatgcaaggttaccaactgttgctccttccactgttcgctcaagctgctaatctgcatctgagcttcatcagggatgtcatcctgaatgccgacgaatggggtatatctgcagctacacttcgcacttacagggactacctgaagaactacacgcgcgactactcgaactactgcatcaacacctatcagtccgccttcaaaggcctgaacacgaggcttcatggtacgttggagtttcggacgtacatgttcctgaacgtgttcgagtatgtctccatctggtcactcttcaagtaccagtcattgctggtctcgtcaggtgctaacctgtacgcatcaggatcaggacctcaacagacgcaatcgttcacgtctcaagactggccattcctgtatagcttgttccaagtcaactccaactacgtgctgaacggcttctctggtgctaggttgtccaacactttcccaaacatcggtggacttccaggaagcactacgactcatgcactgcttgctgcaagggtcaactactctggaggtatctcatctggtgacattggagcttcaccgttcaaccagaacttcaactgcagcacattccttccacctttgcttacgccattcgttagatcatggcttgactctggatctgatagggaaggagtcgctactgtgaccaactggcagacagagtcattcgagacaacactcggtcttcgctcaggagcattcacagcaagaggcaacagcaactacttcccagactacttcattcgcaacatctctggagttcctcttgtcgttaggaacgaggaccttcgcagacctctgcactacaatgagatcaggaacattgcctcaccttcaggtacacctggtggagcaagggcttacatggtctcagttcacaaccgcaagaacaacatccatgcagttcatgagaacggatcgatgatccacttggcacctaacgactacactggattcacgatctcacctatccatgctactcaggtgaacaaccagactcgcactttcatcagcgagaagttcggcaaccaaggcgattctctgaggtttgagcagaacaacacgactgcaaggtacactctcagaggtaacggcaactcgtacaacctgtacttgcgcgtctccagcataggcaactcaacgatccgcgttaccatcaacggtcgcgtttacactgctacaaacgtcaacacgaccactaacaacgatggtgtcaacgacaatggtgctcgcttcagcgacatcaacatcggtaacgttgtcgcaagcagcaactctgacgttcctctggacatcaacgttacgttcaactctggaacacagttcgatttgatgaacaccatgctggttccgacgaacatcagcccattgtactgagttgcgtggaccgaagcttgcgcgcctaggtttttgtgatctgatgataagtggttggttcgtgtctcatgcacttgggaggtgatctatttcacctggtgtagtttgtgtttccgtcagttggaaaaacttatccctatcgatttcgttttcattttctgcttttcttttatgtaccttcgtttgggcttgtaacgggcctttgtatttcaactctcaataataatccaagtgcatgttaaacaatttgtcatctgtttcggctttgatatactactggtgaagatgggccgtactactgcatcacaacgaaaaataataataagatgaaaaacttgaagtggaaaaaaaaaaaaacttgaatgttcactactactcattgaccataatgtttaacatacatagctcaatagtatttttgtgaatatggcaacacaaacagtccaaaacaattgtctcttactataccaaaccaagggcgccgcttgtttgccactctttgtgtgcaatagtgtgattaccacatctccacattcaatatattccctgaattatctgacgattttgatggctcactgttttcccaagtcttgaattgtcttctgtgcgccagtcaaatgcatatgtgttgagtttatcttttaaatatcaagcttttgtttttaacttttgtttgtaaccaaaaactcacagtaggagtttgatcacataattttatgtttgcctttgcaatttctagtgagtctttgattaaaagcttgaaaagaaaatgcagccaagcttaccaagtaagttatgtgtattaaccagaggaagagagaatcttgcaaaatttcaacaaacacaaaaagaagtattactacgattggtggagaaagaaaacgattccaaatcttgaactgttgttgtaaaagcatagcagaaagtgggagacaaccgaaatagaaatgactataacttaatttaatgttatcattataatttcttctagcaaatatttagaaagtaaatatcacatcaacctttaatgtaattaagctttctctttttgattcatgtgagatgaaaagaaaaaaaagaagagaaaagtgtagaaaacacatcatttctaagctgaaggtacatagtacccttgtacttttggtttcacctgcatagagaaaacccacaagaatatgacagtctgatttgtcagtctcattctcaagcaacatttctctatccgttactttcatggtgaataacacaatccatcatcaatactttgtgttactcagaaactgaaagttattccgagtcttgcatatctttgggcctactcgtttttctaccattattgctgattgttaagctctcgctacttgaatcggcattgttggagtgggaaggttcaaaaaattggagttatgactagttgtctctttctatgtacgatggagaaaatgaataaacaactgagaaaatggctcttgtttagttgatgatgctcttaagctttccactggttgccatatatgatttgggcatttcactttgatcttaatgggccttgtaaggcccaagactcatgattatctttagttgatgctcttaattaggtgtgggcaaataattcaaactgtatgtacccgaccaaaaccaaagcaaaaataatcgaaccaaaccgaaaatttaaaaataaccgaatgaaaactaaatcctataactgaaagaactgaaaccgaatcaaaatatttaatgtaaccaaaaatatccgaaatataattatattgtcaaaaatattaataatttctagattaaataattaaaaatacttaaaaatttatataaaatagtaaaaatactcgaaaataaccacaaatattcaaaaacaaccgaaatatcccaaaatattcaaagcaaaataaccgaatggataccaaattttaaaaccgaaaaaactggaacaaaaccagaatcgaaccaaaatttcaaaaatcgaataaatactaaactttagaacaaaaaaaaacgataaccgaatgtatacgaaccaaagccgaattagataaccgaacgtccaggactactcttaatctttccgccacttatgatttgggctattactttgtttataatgagccttttcaagctcaagttcatgattgtccgtgagatgagaaactgacttgttggattcgaaaccctagctagtattggttaatacttaatacataaatgacctgcattgacatcatcatccaagaaaataaaaattgtatgcttgagatatttagttttcctagctaggttttctttattttagtaccgaatctttaggtgtgccacgttaatttagacccattttttcatacttaccaactgagtctagtttaatcatgactataatcgtataaaatgattcagtcgacgtcattgcgaacgtatataaaatcatccaaattgacgtcattccaaagaggtaagcatgcttatctaagagtccgagcatactaaacaagacgacattttatttgcactctaaatcaaattttgtattgcctaaagaaaaacaatcaaactcaagtttcttaaaattaatttcattcaaactaatcactttcaatatctcacatattattcatgccatttctatttgtctaaacatgatttaaaaaaaaagtaaaatacaaagattactatgcaaaaactctataaaaaaaaattcaaatttcttatttatttgtgacatcaaattttcaaaataatttttttaattatcggttgatccggtcagtcgataaaaacataaactttcagcgaccgttaaaactttcctactaccgatttagagaaaatcttagcttgaaacgtaattgtaacctgccttcatgcaagtcgcaagatatgtcatcctaagttgtatatgttttctcaaaagatgtatttacttgagaaaatacgtttcaacgttgatggacaaccaattaagaatcaagcacctttcgtaatcaatttaggcttatcgtctaaggtatactgatttacgacagttgactagacttataaggaacaaaataatagaataatttcgtcaagaaaaattgattttggactcatactttacataatattttactcttaaatttatttaagtggctcctcgcatgatcccaaagagcaagcctagactatatggaaaagtttctaaacacttcacctaatcatagagactaagatggtaattcgtaaacgacaaagcctagtgacactgtccattgtaaaattccacatcatattagtattaaacatatacatgtagtttcctgaacacatgtagtatcaaacacacttcgtggcttcttcctcgaaatcgaggcctaggcttaaggtttaaacagcccgggcgcgcccggaccgggccatctaggccccttagggagctctcgcgacgtcaatcgagtacgtacgtaagggcgacaccccctaattagcccgggtctagagtcgacagatctccatggatccgttaacggccactttgtacaagaaagctgggtgcccgggaataagtgactagggtcacgtgaccctagtcacttaggtgaccaagcttcggccgcaggatagaggacatcctggacctactgaacgtcagcaatgacgactgaaagattcccaggacaccggcggaagtggtggacccagtctaggtgcgatgcttagtcgcgcacgatgactatgtcggaaggcatctttgctttcggcaaactttagtaatactttaaggaaagtattgtacaagttaggtgcagagacaataatgcacccagctttagctttgtttatggaattattgtgtcggttgcattattggatgcctgcgtgcaccctaagcaatccccggccctcttctctataagaggagcccttgcaatcagttgcaagcatgcaagtttcccactgcaagcttacttctgagtttgagttcaagttcaataaaattcaagctttcctcttacattctgttcttgaaaggttcgatctaatcgagcgagtagagaacaagatcttttgggatttccgccgttccggatcttcgatatcccggactggcgccaggtccgccttgtttctcctctgtctcttgatctgactaatcttggtttatgattcgttgagtaattttggggaaagcttcgtccacagtttttttttcgatgaacagtgccgcagtggcgctgatcttgtatgctatcctgcaatcgtggtgaacttatttcttttatatccttcactcccatgaaaaggctagtaatctttctcgatgtaacatcgtccagcactgctattaccgtgtggtccatccgacagtctggctgaacacatcatacgatattgagcaaagatcgatctatcttccctgttctttaatgaaagacgtcattttcatcagtatgatctaagaatgttgcaacttgcaaggaggcgtttctttctttgaatttaactaactcgttgagtggccctgtttctcggacgtaaggcctttgctgctccacacatgtccattcgaattttaccgtgtttagcaagggcgaaaagtttgcatcttgatgatttagcttgactatgcgattgctttcctggacccgtgcagctggcgccttgggatccatgggccacaacaacccgaacatcaacgagtgcatcccgtacaactgcctgtccaacccggaggtggaggtgcttggaggcgagagaatcgagaccggctacactcccatcgacatcagcctcagccttacccagttcctgctctcggagttcgtgccaggagcaggtttcgtgctgggactggtcgacgtgatctggggcatcttcggtccgtcccaatgggatgcgttcctggttcagatcgagcagctgatcaaccagcgcatcgaggagttcgccaggaaccaggccatctctagggtcgagggcctcagcaacctgtaccagatctacgcagagtccttcagagagtgggaggccgatccgaccaatccagcgctcaaggaggagatgcgcacgcagttcaacgacatgaactccgctctgacgacagccattccgctgtttgcggtccagaactaccaggtgccgctgcttagcgtgtacgtccaggctgctaacctccacctgtcggttcttcgggacgtgtcagtgttcggccagaggtggggattcgacgctgcgacgatcaactcgcgctacaacgacctcaccaggctcatcgggaactacacagaccacgcagtgcgctggcacaacaccgggttggagcggatatggggcccggactcgagagattggattcggtacaaccagttccgccgcgagctgaccctcacggtgctggacatcgtgtcgctgttcccgaactacgactcgcgcacgtacccgatccgcacggcgagccaactgaccagggagatctacaccaacccggttctcgagaacttcgacggcagctttcgcggaagcgcgcaaggcatcgaaggttcgatccgctcgccgcacctgatggacatactcaacagcatcaccatctacacggacgcgcacagaggcgagtactactggagcggacaccagatcatggcgagccctgtcggcttctctggaccagagttcacattcccgctgtacggcacgatgggtaacgctgctccgcaacagaggatcgttgctcagctcggccaaggcgtctacagaaccctgtcctcgactctgtaccggaggccgttcaacatcggcatcaacaaccagcagctttccgtccttgacggtacggagttcgcgtatggcacctcatccaacctgccttccgccgtttaccggaagtccgggacagtggacagcctcgacgagatcccgccgcagaacaacaacgtgcctccaaggcaaggcttctctcacaggctctcacacgtgtcgatgttccgctctgggttcagcaactcctccgtctccatcatccgcgctcccatgttctcgtggattcacaggagcgccgagttcaacaacacgatcgacccggagcgcatcaaccagatcccgctgaccaagagcacgaacctcggctcaggcacctctgtggtcaaaggacccggtttcactggcggcgacatcttgaggaggacaagcccagggcagatctccacgcttcgcgtcaacatcacagctccgctgtcccagcgctaccgcgttcggatcaggtacgcctcgacgaccaacctccaattccacacctcgatcgatgggaggccgatcaaccagggcaacttctccgcgacaatgtcctccggcagcaacttgcagagcggttccttccgcaccgtgggcttcaccacgccgttcaacttcagcaacgggtcctctgtcttcaccctgtcggcacatgtgttcaacagcgggaacgaggtctacatcgaccgcatcgagtttgtgccagccgaggttacgtttgaagcggagtacgacctggagcgcgcgcagaaagtggtcaacgcgctgttcacgtcctcgaaccagatcgggctcaagaccgacgtgacggactaccacatcgaccaggtgtccaacctcgtggactgcctgtccgacgagttctgcctcgacgagaagcgcgaactgtccgagaaggtgaagcacgcgaagcggctgtctgacgagcggaaccttctgcaagacccgaacttcagaggtatcaacaggcaacctgaccgcgggtggcgcggatcgacggacatcacgatccagggcggcgacgacgtgttcaaggagaactacgttacactgcccggcacagtggacgagtgttacccgacctacctgtaccagaagatcgacgagtcgaagctcaaggcgtacacgaggtacgagcttcgcggctacatcgaggactcgcaagacctggagatctacctgatccgctacaacgccaagcacgagatcgtgaacgtgcctggtactggttcactgtggccactgagcgcgcaaagcccgattgggaagtgcggtgaacccaacaggtgcgctcctcacctggaatggaatccggacctggattgttcttgccgcgatggcgagaaatgcgcgcaccactcccaccacttcaccctggacatcgacgtcggttgcaccgatctcaacgaggacttgggcgtgtgggtgatcttcaagatcaagacccaggatgggcacgccaggctcggcaacctggagttcctggaggagaagcctctgcttggtgaagcgcttgccagagtcaagagggcggagaagaagtggcgcgacaagcgcgagaagctccagctggagacgaacatcgtctacaaggaggccaaggagtccgtcgacgccctctttgtgaacagccagtacgaccggctccaggtggacacgaacatcgccatgatccatgcagccgacaagcgggttcacaggatcagggaggcttatcttccggagctgagcgtcataccgggcgtgaacgctgcgatcttcgaggagcttgagggccggatcttcacggcttacagcctctacgacgcgaggaacgtgatcaagaacggcgacttcaacaacggcctgctctgctggaacgtcaagggccacgttgacgtcgaggagcagaacaatcaccggagcgtgctggtgatccctgagtgggaagccgaggtgtctcaggaggtcagggtctgtcctggacgcggatacatccttcgcgtcacagcctacaaggagggctatggcgagggctgcgtcaccattcacgagatcgaggacaacaccgacgagctgaagttcagcaattgcgtcgaggaggaggtgtacccgaacaacaccgtcacctgcaacaactacacgggcacacaggaggagtatgagggcacctacacctctcgcaaccagggctacgatgaagcgtacggcaacaacccatcagttcccgccgactacgcctccgtctacgaggagaagtcgtacaccgacggcagacgcgagaatccttgtgagtccaacagaggctacggcgactacacgccactgccggctggatatgtgaccaaggacctggagtacttcccggagaccgacaaggtgtggatcgagatcggcgagaccgagggaaccttcatcgtcgacagcgtcgagctgctcctgatggaggagtaggttaattcgattactagtgtttttctcagacagttttctaaaaaaagggcgtttctggggaagttcgagatggttcgtaaggtgttactggctcctgtgaaccaatacatgatactgccatgataagggttataattagtcaagcagagtaagaagaaacaacagtagcagtgactccgattcctgaagatgagtcatatttgtcttgtgctcctgctgtatgaaatggatcgcatgtgtatattcgtcgccgcgccgcactggtgtaacctgttgcctcagagtttgcttttagctggttctgttttaaaaataagtactgttttttggttggctgcaagccattctgaacttcagtttaccaattgtttttatgttgtggttgaatattttaattttttatttaatgtttggttctttttttatatatatttgcaaaaatgatacaagtggtcaagttttcatatagtatgggctctatttcctagagctctacctctaggaacgaattttgtggaggttttcttttggctagttaggcaaagtccccatatcttgcaggctaaatcaagaagaagctctgtcaaacagttttttttactgaaaagtgattaaagagtagtttctcctagatcacttcagagtttatcctagagaatcatgggaatcaaattcagttagaggatcatttcttacaaagaatcaactttcgtagagaatctaaagcagaaagagctttgacaaacttacccttagagcaattccaacattctcgcgtgagtttcttcgcgccgttgttttgcggtgacttcatctggacgtcccgcgacatagagacgcttgtattgatcatgagagcttgtgtggtcatacacaatataattgttaaagatgaaagagatgtggaccttaatgagcgattcgactttgatggtgaaaatgtgcaaccttctcatggtatttctactcgcacactagctgaatttattgaagctcataaaaagatccgagacaaagaaatacattttcaattgaaagaagacctaatcaagcacttatgggaattcctaggcttaaggtttaaacagccccctccggcggtgtcccccactgaagaaactatgtgctgtagtatagccgctggctagctagctagttgagtcatttagcggcgatgattgagtaataatgtgtcacgcatcaccatgcatgggtggcagtctcagtgtgagcaatgacctgaatgaacaattgaaatgaaaagaaaaaagtattgttccaaattaaacgttttaaccttttaataggtttatacaataattgatatatgttttctgtatatgtctaatttgttatcatccatttagatatagacgaaaaaaaatctaagaactaaaacaaatgctaatttgaaatgaagggagtatatattgggataatgtcgatgagatccctcgtaatatcaccgacatcacacgtgtccagttaatgtatcagtgatacgtgtattcacatttgttgcgcgtaggcgtacccaacaattttgatcgactatcagaaagtcaacggaagcgctgcagaaacttatctctgttatgaatcagaagaagttcatgtctcgtttcatttaaaactttggtggtttgtgttttggggccttgtaaagcccctgatgaataattgttcaactatgtttccgttcctgtgttatacctttctttctaatgagtaatgacatcaaacttcttctgtattgaaattatgtccttgtgagtctctttatcatcgtttcgtctttacattatatgtgctacttttgtctaatgagcctgaaaagtggctccaatggtacgcactggaagatttgttggcttctggtagatatagcgacagtgttgagcttgtaatatcatgtctcttattgctaaattagttcctttcttaacagaaaccttcaaagtttttgtttttgttttcatttacctaatgtacacatacgctggccatgactaacaacatgtccaggcttagagcatatttttttctagcttaaattgttaacttgtcattcagtaaaatccgagaattgtgaagctctaattgaagctaattcgttttataaagtcagttaaaaagtatactaaattatccaacttttcttcaaaatctcaaaattctatgacaaaacgatagtctttgtttatgtcagtaccacaaagaggtggaaaaaaacaccaaaaaaacaataagcaaactatacactgagaagaaaaataaaagagagctcaatagatgttttatactaacggtagattagatcaaagatccaagctttactctacatagagcagaacccagaatcccttcatatctcttttattctagcaccgataatctactgaaaagaagacacttagagctctgtctctttgtcaaagaagtcccagccgtcatccagaagctccttacgttcattaacagagaattcgacaaagcagcattagtccgttgatcggtggaagaccactcgtcagtgttgagttgaatgtttgatcaataaaatacggcaatgctgtaagggttgttttttatgccattgataatacactgtactgttcagttgttgaactctatttcttagccatgccaagtgcttttcttattttgaataacattacagcaaaaagttgaaagacaaaaaaaaaaacccccgaacagagtgctttgggtcccaagcttctttagactgtgttcggcgttccccctaaatttctccccctatatctcactcacttgtcacatcagcgttctctttccccctatatctccacgctctacagcagttccacctatatcaaacctctataccccaccacaacaatattatatactttcatcttcaactaactcatgtaccttccaatttttttctactaataattatttacgtgcacagaaacttagcaaggagagagagagcggggtgaccaagcttggcgcgccgtcccattctggccgaatttaagtgactagggtcacgtgaccctagtcacttaccggattctggccggagcctgcttttttgtacaaacttgaagctggccttctaggcccggaccgggtgaccaagcttgggccgcgtttaaacttcgaaacgcgtggaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccatgaacaagaacaacaccaagctgagcacccgcgccctgccgagcttcatcgactacttcaacggcatctacggcttcgccaccggcatcaaggacatcatgaacatgatcttcaagaccgacaccggcggcgacctgaccctggacgagatcctgaagaaccagcagctgctgaacgacatcagcggcaagctggacggcgtgaacggcagcctgaacgacctgatcgcccagggcaacctgaacaccgagctgagcaaggagatccttaagatcgccaacgagcagaaccaggtgctgaacgacgtgaacaacaagctggacgccatcaacaccatgctgcgcgtgtacctgccgaagatcaccagcatgctgagcgacgtgattaagcagaactacgccctgagcctgcagatcgagtacctgagcaagcagctgcaggagatcagcgacaagctggacatcatcaacgtgaacgtcctgatcaacagcaccctgaccgagatcaccccggcctaccagcgcatcaagtacgtgaacgagaagttcgaagagctgaccttcgccaccgagaccagcagcaaggtgaagaaggacggcagcccggccgacatcctggacgagctgaccgagctgaccgagctggcgaagagcgtgaccaagaacgacgtggacggcttcgagttctacctgaacaccttccacgacgtgatggtgggcaacaacctgttcggccgcagcgccctgaagaccgccagcgagctgatcaccaaggagaacgtgaagaccagcggcagcgaggtgggcaacgtgtacaacttcctgatcgtgctgaccgccctgcaggcccaggccttcctgaccctgaccacctgtcgcaagctgctgggcctggccgacatcgactacaccagcatcatgaacgagcacttgaacaaggagaaggaggagttccgcgtgaacatcctgccgaccctgagcaacaccttcagcaacccgaactacgccaaggtgaagggcagcgacgaggacgccaagatgatcgtggaggctaagccgggccacgcgttgatcggcttcgagatcagcaacgacagcatcaccgtgctgaaggtgtacgaggccaagctgaagcagaactaccaggtggacaaggacagcttgagcgaggtgatctacggcgacatggacaagctgctgtgtccggaccagagcgagcaaatctactacaccaacaacatcgtgttcccgaacgagtacgtgatcaccaagatcgacttcaccaagaagatgaagaccctgcgctacgaggtgaccgccaacttctacgacagcagcaccggcgagatcgacctgaacaagaagaaggtggagagcagcgaggccgagtaccgcaccctgagcgcgaacgacgacggcgtctacatgccactgggcgtgatcagcgagaccttcctgaccccgatcaacggctttggcctgcaggccgacgagaacagccgcctgatcaccctgacctgtaagagctacctgcgcgagctgctgctagccaccgacctgagcaacaaggagaccaagctgatcgtgccaccgagcggcttcatcagcaacatcgtggagaacggcagcatcgaggaggacaacctggagccgtggaaggccaacaacaagaacgcctacgtcgaccacaccggcggcgtgaacggcaccaaggccctgtacgtgcacaaggacggcggcatcagccagttcatcggcgacaagctgaagccgaagaccgagtacgtgatccagtacaccgtgaagggcaagccatcgattcacctgaaggacgagaacaccggctacatccactacgaggacaccaacaacaacctggaggactaccagaccatcaacaagcgcttcaccaccggcaccgacctgaagggcgtgtacctgatcctgaagagccagaacggcgacgaggcctggggcgacaacttcatcatcctggagatcagcccgagcgagaagctgctgagcccggagctgatcaacaccaacaactggaccagcaccggcagcaccaacatcagcggcaacaccctgaccctgtaccagggcggcaggggcatcctgaagcagaacctgcagctggacagcttcagcacctaccgcgtgtacttcagcgtgagcggcgacgccaacgtgcgcatccgcaactcccgcgaggtgctgttcgagaagaggtacatgagcggcgccaaggacgtgagcgagatgttcaccaccaagttcgagaaggacaacttctacatcgagctgagccagggcaacaacctgtacggcggcccgatcgtgcacttctacgacgtgagcatcaagtaggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgacctcgaggcctaggcttaaggtttaaacagcccgggcgcgccggtaccgagctcgaattcggtaacccggtccgggccattctggccgtaccgagctcgaattcggcccaacttttctatacaaagttgatagcgataaatcctgaggatctggtcttcctaaggacccgggatatcggaccgattaaactttaattcggtccgataacttcgtatagcatacattatacgaagttatacctggtggcgccgctagcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatcaattcgctagcgaagttcctattccgaagttcctattctctagaaagtataggaacttcagatccaccgggatccacacgacaccatgtcccccgagcgccgccccgtcgagatccgcccggccaccgccgccgacatggccgccgtgtgcgacatcgtgaaccactacatcgagacctccaccgtgaacttccgcaccgagccgcagaccccgcaggagtggatcgacgacctggagcgcctccaggaccgctacccgtggctcgtggccgaggtggagggcgtggtggccggcatcgcctacgccggcccgtggaaggcccgcaacgcctacgactggaccgtggagtccaccgtgtacgtgtcccaccgccaccagcgcctcggcctcggctccaccctctacacccacctcctcaagagcatggaggcccagggcttcaagtccgtggtggccgtgatcggcctcccgaacgacccgtccgtgcgcctccacgaggccctcggctacaccgcccgcggcaccctccgcgccgccggctacaagcacggcggctggcacgacgtcggcttctggcagcgcgacttcgagctgccggccccgccgcgcccggtgcgcccggtgacgcagatctgagtcgaaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccctagcgtatacgaagttcctattccgaagttcctattctccagaaagtataggaacttctgtacacctgagctgattccgatgacttcgtaggttcctagctcaagccgctcgtgtccaagcgtcacttacgattagctaatgattacggcatctaggaccgactagctaactaactagtacgtagaattaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgcca

SEQ ID NO: 2 - DNA, 26 bp, Artificial Sequence
  primer
cttgtgagtccaacagaggctacggc

SEQ ID NO: 3 - DNA, 28 bp, Artificial Sequence
  primer
ggttcacaggagccagtaacaccttacg

SEQ ID NO: 4 - DNA, 25 bp, Artificial Sequence
  primer
ccgctgtatcacaagggctggtacc

SEQ ID NO: 5 - DNA, 25 bp, Artificial Sequence
  primer
ggagcccgtgtagagcatgacgatc

SEQ ID NO: 6 - DNA, 23 bp, Artificial Sequence
  primer
gcaagaacccgaagaaactcatt

SEQ ID NO: 7 - DNA, 19 bp, Artificial Sequence
  primer
gtcgtttcccgccttcagt

SEQ ID NO: 8 - DNA, 23 bp, Artificial Sequence
  probe
tattgagacaaacactgatagtt

SEQ ID NO: 9 - DNA, 76 bp, Artificial Sequence
  amplicon
gcaagaacccgaagaaactcattctatttagtattgagacaaacactgatagtttaaactgaaggcgggaaacgac

SEQ ID NO: 10 - DNA, 23 bp, Artificial Sequence
  primer
ttggactagaaatctcgtgctga

SEQ ID NO: 11 - DNA, 22 bp, Artificial Sequence
  primer
gctacatagggagccttgtcct

SEQ ID NO: 12 - DNA, 23 bp, Artificial Sequence
  probe
caatccacacaaacgcacgcgta

SEQ ID NO: 13 - DNA, 79 bp, Artificial Sequence
  amplicon
ttggactagaaatctcgtgctgattaattgttttacgcgtgcgtttgtgtggattgtaggacaaggctccctatgtagc

SEQ ID NO: 14 - DNA, 25250 bp, Artificial Sequence
  5′ flanking - event - 3′ flanking
ttctctggagtttcattatttcattgtacatatgaaaagaaacttacatcatataggagttttccaaaaatatgtatataatattgttgtataaaacataaaagcattattaaaccttgcagaggagcttccacatgattccaactgcatatttgattgaatttttcaagtcatctaaaaagaacaacatttaaagcaaaaactcgagtcaaattgatgtaacattagggattaccagatcccaatgcacgacgattgacacggtcgttggtgaggagtgacaaggtcagtggcttggggataggtgcgcttggttgatgtcagataattgagttcggcagctatgtgaggatcagtattgaggcacacaacctatggcggagcaagaacccgaagaaactcattctatttagtattgagacaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcggttactaccggctggatggcggggccttgatcgtgcaccgccggcgtccggactaactaactagtcgagctagttaccctatgaggtgacatgaagcgctcacggttactatgacggttagcttcacgactgttggtggcagtagcgtacgacttagctatagttccggacttacccttaagataacttcgtatagcatacattatacgaagttatgggcccaccggtggtaccgagctcgtttaaacgctcttcaactggaagagcggttaccagagctggtcacctttgtccaccaagatggaactggcgcgcctcattaattaagtcagcggccgctctagttgaagacacgttcatgtcttcatcgtaagaagacactcagtagtcttcggccagaatggccatctggattcagcaggcctagaaggccatttatctatcaactttgtataataaagttgcccggtccttaggcggaccgggccatctaggccgcggccgcactgtcaagctattattagcttctttaataagtccaatgtgaacaaaccgtctagggttagatggattgctttcacagatttccttactggtctaggaatccctgtaaatatagagcacatagatggaaaaaataaccatctggctgatgctctgtccagattagtaactggttttgtttttgcagaaccacaatgtcaagacaagttccaggacgatttagggaaattggaagcagctcttcaggagaagaaagaggctccgcaagcaatgcacgtagaatatgtctccctgttgatcagatcagcggaccgcattacccgctcgctctgctttatgagggactcgtctcacagcagaatttactcatgcaggccaggcaaagaaccaatgaaggccttaatctgcgaacagaagtcatgccaatccaaaggcgacttagggaatacgaggactgtgcactccaagagtgcattcaatcagcaagacaactggtggccctccaccagcacaaactcgcttacatcagaagcaaagctacaagggacaacgcatatgccgataggctacccacatgcaatcgggaccacgagcaactgtgtgaagtggtcgagctattagaaggaatctcggaaagaatcagcgatacagctgtctaggacagctggcttcaattatggagcgtgatggacccccccgcaataatccaaagtttggtgtgcttttagtagtgcgtctttatggaccactactttattgtaataatcgatgctttttgtagtgcgctcttcgtgcgctctactttatgcttttgcttttgtaagtgcgctgtaagtgcgcctgtctttcttcagatgcttatcctttaagcatcttttgctttttgcgtggcatcctttagttcacaatttaaagaatgacgatggggcccaagatgtgcacccggttctctaaattgcctatataaggatatgccatagccttgtttttgcaagtcaggaatacctgagcataacttggctaagcaaaagtttgtaagtgttctaagctttcatttgtaaactttctgtttggttttaataaaatctctcgtcaatcgttgtgaacatatattgtttgtttgtattgttgtatcttatttgttgtggtgataaggatcttcgatatcccggactggcgccaggtccgccttgtttctcctctgtctcttgatctgactaatcttggtttatgattcgttgagtaattttggggaaagcttcgtccacagtttttttttcgatgaacagtgccgcagtggcgctgatcttgtatgctatcctgcaatcgtggtgaacttatttcttttatatccttcactcccatgaaaaggctagtaatctttctcgatgtaacatcgtccagcactgctattaccgtgtggtccatccgacagtctggctgaacacatcatacgatattgagcaaagatcgatctatcttccctgttctttaatgaaagacgtcattttcatcagtatgatctaagaatgttgcaacttgcaaggaggcgtttctttctttgaatttaactaactcgttgagtggccctgtttctcggacgtaaggcctttgctgctccacacatgtccattcgaattttaccgtgtttagcaagggcgaaaagtttgcatcttgatgatttagcttgactatgcgattgctttcctggacccgtgcagctggcgccttgggatccatggctgcgaccactctcacgagcgctctcccaggagcctttagcagctctcagagaccttcggctccgttcaacctccagaggagccctagagtcctcagacgcttcaaccgcaagaccggtagacagccacgcggtctcgtcagagctgctaaggctcagcgctctggtaccagatccatgggcaactccgttctcaattccggaaggactacgatctgtgatgcgtacaacgttgcagctcatgatccgttctcattccagcacaagtcacttgacactgttcagagggagtggactgagtggaagaagaacaaccattcgctgtatctcgatccgatcgttggaactgtggcttcattcctgctcaagaaggtcggttctctcgttggtaagaggattctctcggaactcaggaacttgatcttcccatctggtagcacaaacctcatgcaggacatacttagggaaactgagcagttcctgaaccaacgccttgacactgataccttggcaagggtcaatgctgagttgacaggtcttcaagcgaacgttgaggagttcaatcgccaagttgacaacttccttaaccctaaccggaatgccgttcctctgtctatcacgtcatctgtcaacacgatgcagcagctgttcttgaaccggcttcctcaattccagatgcaaggttaccaactgttgctccttccactgttcgctcaagctgctaatctgcatctgagcttcatcagggatgtcatcctgaatgccgacgaatggggtatatctgcagctacacttcgcacttacagggactacctgaagaactacacgcgcgactactcgaactactgcatcaacacctatcagtccgccttcaaaggcctgaacacgaggcttcatggtacgttggagtttcggacgtacatgttcctgaacgtgttcgagtatgtctccatctggtcactcttcaagtaccagtcattgctggtctcgtcaggtgctaacctgtacgcatcaggatcaggacctcaacagacgcaatcgttcacgtctcaagactggccattcctgtatagcttgttccaagtcaactccaactacgtgctgaacggcttctctggtgctaggttgtccaacactttcccaaacatcggtggacttccaggaagcactacgactcatgcactgcttgctgcaagggtcaactactctggaggtatctcatctggtgacattggagcttcaccgttcaaccagaacttcaactgcagcacattccttccacctttgcttacgccattcgttagatcatggcttgactctggatctgatagggaaggagtcgctactgtgaccaactggcagacagagtcattcgagacaacactcggtcttcgctcaggagcattcacagcaagaggcaacagcaactacttcccagactacttcattcgcaacatctctggagttcctcttgtcgttaggaacgaggaccttcgcagacctctgcactacaatgagatcaggaacattgcctcaccttcaggtacacctggtggagcaagggcttacatggtctcagttcacaaccgcaagaacaacatccatgcagttcatgagaacggatcgatgatccacttggcacctaacgactacactggattcacgatctcacctatccatgctactcaggtgaacaaccagactcgcactttcatcagcgagaagttcggcaaccaaggcgattctctgaggtttgagcagaacaacacgactgcaaggtacactctcagaggtaacggcaactcgtacaacctgtacttgcgcgtctccagcataggcaactcaacgatccgcgttaccatcaacggtcgcgtttacactgctacaaacgtcaacacgaccactaacaacgatggtgtcaacgacaatggtgctcgcttcagcgacatcaacatcggtaacgttgtcgcaagcagcaactctgacgttcctctggacatcaacgttacgttcaactctggaacacagttcgatttgatgaacaccatgctggttccgacgaacatcagcccattgtactgagttgcgtggaccgaagcttgcgcgcctaggtttttgtgatctgatgataagtggttggttcgtgtctcatgcacttgggaggtgatctatttcacctggtgtagtttgtgtttccgtcagttggaaaaacttatccctatcgatttcgttttcattttctgcttttcttttatgtaccttcgtttgggcttgtaacgggcctttgtatttcaactctcaataataatccaagtgcatgttaaacaatttgtcatctgtttcggctttgatatactactggtgaagatgggccgtactactgcatcacaacgaaaaataataataagatgaaaaacttgaagtggaaaaaaaaaaaaacttgaatgttcactactactcattgaccataatgtttaacatacatagctcaatagtatttttgtgaatatggcaacacaaacagtccaaaacaattgtctcttactataccaaaccaagggcgccgcttgtttgccactctttgtgtgcaatagtgtgattaccacatctccacattcaatatattccctgaattatctgacgattttgatggctcactgttttcccaagtcttgaattgtcttctgtgcgccagtcaaatgcatatgtgttgagtttatcttttaaatatcaagcttttgtttttaacttttgtttgtaaccaaaaactcacagtaggagtttgatcacataattttatgtttgcctttgcaatttctagtgagtctttgattaaaagcttgaaaagaaaatgcagccaagcttaccaagtaagttatgtgtattaaccagaggaagagagaatcttgcaaaatttcaacaaacacaaaaagaagtattactacgattggtggagaaagaaaacgattccaaatcttgaactgttgttgtaaaagcatagcagaaagtgggagacaaccgaaatagaaatgactataacttaatttaatgttatcattataatttcttctagcaaatatttagaaagtaaatatcacatcaacctttaatgtaattaagctttctctttttgattcatgtgagatgaaaagaaaaaaaagaagagaaaagtgtagaaaacacatcatttctaagctgaaggtacatagtacccttgtacttttggtttcacctgcatagagaaaacccacaagaatatgacagtctgatttgtcagtctcattctcaagcaacatttctctatccgttactttcatggtgaataacacaatccatcatcaatactttgtgttactcagaaactgaaagttattccgagtcttgcatatctttgggcctactcgtttttctaccattattgctgattgttaagctctcgctacttgaatcggcattgttggagtgggaaggttcaaaaaattggagttatgactagttgtctctttctatgtacgatggagaaaatgaataaacaactgagaaaatggctcttgtttagttgatgatgctcttaagctttccactggttgccatatatgatttgggcatttcactttgatcttaatgggccttgtaaggcccaagactcatgattatctttagttgatgctcttaattaggtgtgggcaaataattcaaactgtatgtacccgaccaaaaccaaagcaaaaataatcgaaccaaaccgaaaatttaaaaataaccgaatgaaaactaaatcctataactgaaagaactgaaaccgaatcaaaatatttaatgtaaccaaaaatatccgaaatataattatattgtcaaaaatattaataatttctagattaaataattaaaaatacttaaaaatttatataaaatagtaaaaatactcgaaaataaccacaaatattcaaaaacaaccgaaatatcccaaaatattcaaagcaaaataaccgaatggataccaaattttaaaaccgaaaaaactggaacaaaaccagaatcgaaccaaaatttcaaaaatcgaataaatactaaactttagaacaaaaaaaaacgataaccgaatgtatacgaaccaaagccgaattagataaccgaacgtccaggactactcttaatctttccgccacttatgatttgggctattactttgtttataatgagccttttcaagctcaagttcatgattgtccgtgagatgagaaactgacttgttggattcgaaaccctagctagtattggttaatacttaatacataaatgacctgcattgacatcatcatccaagaaaataaaaattgtatgcttgagatatttagttttcctagctaggttttctttattttagtaccgaatctttaggtgtgccacgttaatttagacccattttttcatacttaccaactgagtctagtttaatcatgactataatcgtataaaatgattcagtcgacgtcattgcgaacgtatataaaatcatccaaattgacgtcattccaaagaggtaagcatgcttatctaagagtccgagcatactaaacaagacgacattttatttgcactctaaatcaaattttgtattgcctaaagaaaaacaatcaaactcaagtttcttaaaattaatttcattcaaactaatcactttcaatatctcacatattattcatgccatttctatttgtctaaacatgatttaaaaaaaaagtaaaatacaaagattactatgcaaaaactctataaaaaaaaattcaaatttcttatttatttgtgacatcaaattttcaaaataatttttttaattatcggttgatccggtcagtcgataaaaacataaactttcagcgaccgttaaaactttcctactaccgatttagagaaaatcttagcttgaaacgtaattgtaacctgccttcatgcaagtcgcaagatatgtcatcctaagttgtatatgttttctcaaaagatgtatttacttgagaaaatacgtttcaacgttgatggacaaccaattaagaatcaagcacctttcgtaatcaatttaggcttatcgtctaaggtatactgatttacgacagttgactagacttataaggaacaaaataatagaataatttcgtcaagaaaaattgattttggactcatactttacataatattttactcttaaatttatttaagtggctcctcgcatgatcccaaagagcaagcctagactatatggaaaagtttctaaacacttcacctaatcatagagactaagatggtaattcgtaaacgacaaagcctagtgacactgtccattgtaaaattccacatcatattagtattaaacatatacatgtagtttcctgaacacatgtagtatcaaacacacttcgtggcttcttcctcgaaatcgaggcctaggcttaaggtttaaacagcccgggcgcgcccggaccgggccatctaggccccttagggagctctcgcgacgtcaatcgagtacgtacgtaagggcgacaccccctaattagcccgggtctagagtcgacagatctccatggatccgttaacggccactttgtacaagaaagctgggtgcccgggaataagtgactagggtcacgtgaccctagtcacttaggtgaccaagcttcggccgcaggatagaggacatcctggacctactgaacgtcagcaatgacgactgaaagattcccaggacaccggcggaagtggtggacccagtctaggtgcgatgcttagtcgcgcacgatgactatgtcggaaggcatctttgctttcggcaaactttagtaatactttaaggaaagtattgtacaagttaggtgcagagacaataatgcacccagctttagctttgtttatggaattattgtgtcggttgcattattggatgcctgcgtgcaccctaagcaatccccggccctcttctctataagaggagcccttgcaatcagttgcaagcatgcaagtttcccactgcaagcttacttctgagtttgagttcaagttcaataaaattcaagctttcctcttacattctgttcttgaaaggttcgatctaatcgagcgagtagagaacaagatcttttgggatttccgccgttccggatcttcgatatcccggactggcgccaggtccgccttgtttctcctctgtctcttgatctgactaatcttggtttatgattcgttgagtaattttggggaaagcttcgtccacagtttttttttcgatgaacagtgccgcagtggcgctgatcttgtatgctatcctgcaatcgtggtgaacttatttcttttatatccttcactcccatgaaaaggctagtaatctttctcgatgtaacatcgtccagcactgctattaccgtgtggtccatccgacagtctggctgaacacatcatacgatattgagcaaagatcgatctatcttccctgttctttaatgaaagacgtcattttcatcagtatgatctaagaatgttgcaacttgcaaggaggcgtttctttctttgaatttaactaactcgttgagtggccctgtttctcggacgtaaggcctttgctgctccacacatgtccattcgaattttaccgtgtttagcaagggcgaaaagtttgcatcttgatgatttagcttgactatgcgattgctttcctggacccgtgcagctggcgccttgggatccatgggccacaacaacccgaacatcaacgagtgcatcccgtacaactgcctgtccaacccggaggtggaggtgcttggaggcgagagaatcgagaccggctacactcccatcgacatcagcctcagccttacccagttcctgctctcggagttcgtgccaggagcaggtttcgtgctgggactggtcgacgtgatctggggcatcttcggtccgtcccaatgggatgcgttcctggttcagatcgagcagctgatcaaccagcgcatcgaggagttcgccaggaaccaggccatctctagggtcgagggcctcagcaacctgtaccagatctacgcagagtccttcagagagtgggaggccgatccgaccaatccagcgctcaaggaggagatgcgcacgcagttcaacgacatgaactccgctctgacgacagccattccgctgtttgcggtccagaactaccaggtgccgctgcttagcgtgtacgtccaggctgctaacctccacctgtcggttcttcgggacgtgtcagtgttcggccagaggtggggattcgacgctgcgacgatcaactcgcgctacaacgacctcaccaggctcatcgggaactacacagaccacgcagtgcgctggcacaacaccgggttggagcggatatggggcccggactcgagagattggattcggtacaaccagttccgccgcgagctgaccctcacggtgctggacatcgtgtcgctgttcccgaactacgactcgcgcacgtacccgatccgcacggcgagccaactgaccagggagatctacaccaacccggttctcgagaacttcgacggcagctttcgcggaagcgcgcaaggcatcgaaggttcgatccgctcgccgcacctgatggacatactcaacagcatcaccatctacacggacgcgcacagaggcgagtactactggagcggacaccagatcatggcgagccctgtcggcttctctggaccagagttcacattcccgctgtacggcacgatgggtaacgctgctccgcaacagaggatcgttgctcagctcggccaaggcgtctacagaaccctgtcctcgactctgtaccggaggccgttcaacatcggcatcaacaaccagcagctttccgtccttgacggtacggagttcgcgtatggcacctcatccaacctgccttccgccgtttaccggaagtccgggacagtggacagcctcgacgagatcccgccgcagaacaacaacgtgcctccaaggcaaggcttctctcacaggctctcacacgtgtcgatgttccgctctgggttcagcaactcctccgtctccatcatccgcgctcccatgttctcgtggattcacaggagcgccgagttcaacaacacgatcgacccggagcgcatcaaccagatcccgctgaccaagagcacgaacctcggctcaggcacctctgtggtcaaaggacccggtttcactggcggcgacatcttgaggaggacaagcccagggcagatctccacgcttcgcgtcaacatcacagctccgctgtcccagcgctaccgcgttcggatcaggtacgcctcgacgaccaacctccaattccacacctcgatcgatgggaggccgatcaaccagggcaacttctccgcgacaatgtcctccggcagcaacttgcagagcggttccttccgcaccgtgggcttcaccacgccgttcaacttcagcaacgggtcctctgtcttcaccctgtcggcacatgtgttcaacagcgggaacgaggtctacatcgaccgcatcgagtttgtgccagccgaggttacgtttgaagcggagtacgacctggagcgcgcgcagaaagtggtcaacgcgctgttcacgtcctcgaaccagatcgggctcaagaccgacgtgacggactaccacatcgaccaggtgtccaacctcgtggactgcctgtccgacgagttctgcctcgacgagaagcgcgaactgtccgagaaggtgaagcacgcgaagcggctgtctgacgagcggaaccttctgcaagacccgaacttcagaggtatcaacaggcaacctgaccgcgggtggcgcggatcgacggacatcacgatccagggcggcgacgacgtgttcaaggagaactacgttacactgcccggcacagtggacgagtgttacccgacctacctgtaccagaagatcgacgagtcgaagctcaaggcgtacacgaggtacgagcttcgcggctacatcgaggactcgcaagacctggagatctacctgatccgctacaacgccaagcacgagatcgtgaacgtgcctggtactggttcactgtggccactgagcgcgcaaagcccgattgggaagtgcggtgaacccaacaggtgcgctcctcacctggaatggaatccggacctggattgttcttgccgcgatggcgagaaatgcgcgcaccactcccaccacttcaccctggacatcgacgtcggttgcaccgatctcaacgaggacttgggcgtgtgggtgatcttcaagatcaagacccaggatgggcacgccaggctcggcaacctggagttcctggaggagaagcctctgcttggtgaagcgcttgccagagtcaagagggcggagaagaagtggcgcgacaagcgcgagaagctccagctggagacgaacatcgtctacaaggaggccaaggagtccgtcgacgccctctttgtgaacagccagtacgaccggctccaggtggacacgaacatcgccatgatccatgcagccgacaagcgggttcacaggatcagggaggcttatcttccggagctgagcgtcataccgggcgtgaacgctgcgatcttcgaggagcttgagggccggatcttcacggcttacagcctctacgacgcgaggaacgtgatcaagaacggcgacttcaacaacggcctgctctgctggaacgtcaagggccacgttgacgtcgaggagcagaacaatcaccggagcgtgctggtgatccctgagtgggaagccgaggtgtctcaggaggtcagggtctgtcctggacgcggatacatccttcgcgtcacagcctacaaggagggctatggcgagggctgcgtcaccattcacgagatcgaggacaacaccgacgagctgaagttcagcaattgcgtcgaggaggaggtgtacccgaacaacaccgtcacctgcaacaactacacgggcacacaggaggagtatgagggcacctacacctctcgcaaccagggctacgatgaagcgtacggcaacaacccatcagttcccgccgactacgcctccgtctacgaggagaagtcgtacaccgacggcagacgcgagaatccttgtgagtccaacagaggctacggcgactacacgccactgccggctggatatgtgaccaaggacctggagtacttcccggagaccgacaaggtgtggatcgagatcggcgagaccgagggaaccttcatcgtcgacagcgtcgagctgctcctgatggaggagtaggttaattcgattactagtgtttttctcagacagttttctaaaaaaagggcgtttctggggaagttcgagatggttcgtaaggtgttactggctcctgtgaaccaatacatgatactgccatgataagggttataattagtcaagcagagtaagaagaaacaacagtagcagtgactccgattcctgaagatgagtcatatttgtcttgtgctcctgctgtatgaaatggatcgcatgtgtatattcgtcgccgcgccgcactggtgtaacctgttgcctcagagtttgcttttagctggttctgttttaaaaataagtactgttttttggttggctgcaagccattctgaacttcagtttaccaattgtttttatgttgtggttgaatattttaattttttatttaatgtttggttctttttttatatatatttgcaaaaatgatacaagtggtcaagttttcatatagtatgggctctatttcctagagctctacctctaggaacgaattttgtggaggttttcttttggctagttaggcaaagtccccatatcttgcaggctaaatcaagaagaagctctgtcaaacagttttttttactgaaaagtgattaaagagtagtttctcctagatcacttcagagtttatcctagagaatcatgggaatcaaattcagttagaggatcatttcttacaaagaatcaactttcgtagagaatctaaagcagaaagagctttgacaaacttacccttagagcaattccaacattctcgcgtgagtttcttcgcgccgttgttttgcggtgacttcatctggacgtcccgcgacatagagacgcttgtattgatcatgagagcttgtgtggtcatacacaatataattgttaaagatgaaagagatgtggaccttaatgagcgattcgactttgatggtgaaaatgtgcaaccttctcatggtatttctactcgcacactagctgaatttattgaagctcataaaaagatccgagacaaagaaatacattttcaattgaaagaagacctaatcaagcacttatgggaattcctaggcttaaggtttaaacagccccctccggcggtgtcccccactgaagaaactatgtgctgtagtatagccgctggctagctagctagttgagtcatttagcggcgatgattgagtaataatgtgtcacgcatcaccatgcatgggtggcagtctcagtgtgagcaatgacctgaatgaacaattgaaatgaaaagaaaaaagtattgttccaaattaaacgttttaaccttttaataggtttatacaataattgatatatgttttctgtatatgtctaatttgttatcatccatttagatatagacgaaaaaaaatctaagaactaaaacaaatgctaatttgaaatgaagggagtatatattgggataatgtcgatgagatccctcgtaatatcaccgacatcacacgtgtccagttaatgtatcagtgatacgtgtattcacatttgttgcgcgtaggcgtacccaacaattttgatcgactatcagaaagtcaacggaagcgctgcagaaacttatctctgttatgaatcagaagaagttcatgtctcgtttcatttaaaactttggtggtttgtgttttggggccttgtaaagcccctgatgaataattgttcaactatgtttccgttcctgtgttatacctttctttctaatgagtaatgacatcaaacttcttctgtattgaaattatgtccttgtgagtctctttatcatcgtttcgtctttacattatatgtgctacttttgtctaatgagcctgaaaagtggctccaatggtacgcactggaagatttgttggcttctggtagatatagcgacagtgttgagcttgtaatatcatgtctcttattgctaaattagttcctttcttaacagaaaccttcaaagtttttgtttttgttttcatttacctaatgtacacatacgctggccatgactaacaacatgtccaggcttagagcatatttttttctagcttaaattgttaacttgtcattcagtaaaatccgagaattgtgaagctctaattgaagctaattcgttttataaagtcagttaaaaagtatactaaattatccaacttttcttcaaaatctcaaaattctatgacaaaacgatagtctttgtttatgtcagtaccacaaagaggtggaaaaaaacaccaaaaaaacaataagcaaactatacactgagaagaaaaataaaagagagctcaatagatgttttatactaacggtagattagatcaaagatccaagctttactctacatagagcagaacccagaatcccttcatatctcttttattctagcaccgataatctactgaaaagaagacacttagagctctgtctctttgtcaaagaagtcccagccgtcatccagaagctccttacgttcattaacagagaattcgacaaagcagcattagtccgttgatcggtggaagaccactcgtcagtgttgagttgaatgtttgatcaataaaatacggcaatgctgtaagggttgttttttatgccattgataatacactgtactgttcagttgttgaactctatttcttagccatgccaagtgcttttcttattttgaataacattacagcaaaaagttgaaagacaaaaaaaaaaacccccgaacagagtgctttgggtcccaagcttctttagactgtgttcggcgttccccctaaatttctccccctatatctcactcacttgtcacatcagcgttctctttccccctatatctccacgctctacagcagttccacctatatcaaacctctataccccaccacaacaatattatatactttcatcttcaactaactcatgtaccttccaatttttttctactaataattatttacgtgcacagaaacttagcaaggagagagagagcggggtgaccaagcttggcgcgccgtcccattctggccgaatttaagtgactagggtcacgtgaccctagtcacttaccggattctggccggagcctgcttttttgtacaaacttgaagctggccttctaggcccggaccgggtgaccaagcttgggccgcgtttaaacttcgaaacgcgtggaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccatgaacaagaacaacaccaagctgagcacccgcgccctgccgagcttcatcgactacttcaacggcatctacggcttcgccaccggcatcaaggacatcatgaacatgatcttcaagaccgacaccggcggcgacctgaccctggacgagatcctgaagaaccagcagctgctgaacgacatcagcggcaagctggacggcgtgaacggcagcctgaacgacctgatcgcccagggcaacctgaacaccgagctgagcaaggagatccttaagatcgccaacgagcagaaccaggtgctgaacgacgtgaacaacaagctggacgccatcaacaccatgctgcgcgtgtacctgccgaagatcaccagcatgctgagcgacgtgattaagcagaactacgccctgagcctgcagatcgagtacctgagcaagcagctgcaggagatcagcgacaagctggacatcatcaacgtgaacgtcctgatcaacagcaccctgaccgagatcaccccggcctaccagcgcatcaagtacgtgaacgagaagttcgaagagctgaccttcgccaccgagaccagcagcaaggtgaagaaggacggcagcccggccgacatcctggacgagctgaccgagctgaccgagctggcgaagagcgtgaccaagaacgacgtggacggcttcgagttctacctgaacaccttccacgacgtgatggtgggcaacaacctgttcggccgcagcgccctgaagaccgccagcgagctgatcaccaaggagaacgtgaagaccagcggcagcgaggtgggcaacgtgtacaacttcctgatcgtgctgaccgccctgcaggcccaggccttcctgaccctgaccacctgtcgcaagctgctgggcctggccgacatcgactacaccagcatcatgaacgagcacttgaacaaggagaaggaggagttccgcgtgaacatcctgccgaccctgagcaacaccttcagcaacccgaactacgccaaggtgaagggcagcgacgaggacgccaagatgatcgtggaggctaagccgggccacgcgttgatcggcttcgagatcagcaacgacagcatcaccgtgctgaaggtgtacgaggccaagctgaagcagaactaccaggtggacaaggacagcttgagcgaggtgatctacggcgacatggacaagctgctgtgtccggaccagagcgagcaaatctactacaccaacaacatcgtgttcccgaacgagtacgtgatcaccaagatcgacttcaccaagaagatgaagaccctgcgctacgaggtgaccgccaacttctacgacagcagcaccggcgagatcgacctgaacaagaagaaggtggagagcagcgaggccgagtaccgcaccctgagcgcgaacgacgacggcgtctacatgccactgggcgtgatcagcgagaccttcctgaccccgatcaacggctttggcctgcaggccgacgagaacagccgcctgatcaccctgacctgtaagagctacctgcgcgagctgctgctagccaccgacctgagcaacaaggagaccaagctgatcgtgccaccgagcggcttcatcagcaacatcgtggagaacggcagcatcgaggaggacaacctggagccgtggaaggccaacaacaagaacgcctacgtcgaccacaccggcggcgtgaacggcaccaaggccctgtacgtgcacaaggacggcggcatcagccagttcatcggcgacaagctgaagccgaagaccgagtacgtgatccagtacaccgtgaagggcaagccatcgattcacctgaaggacgagaacaccggctacatccactacgaggacaccaacaacaacctggaggactaccagaccatcaacaagcgcttcaccaccggcaccgacctgaagggcgtgtacctgatcctgaagagccagaacggcgacgaggcctggggcgacaacttcatcatcctggagatcagcccgagcgagaagctgctgagcccggagctgatcaacaccaacaactggaccagcaccggcagcaccaacatcagcggcaacaccctgaccctgtaccagggcggcaggggcatcctgaagcagaacctgcagctggacagcttcagcacctaccgcgtgtacttcagcgtgagcggcgacgccaacgtgcgcatccgcaactcccgcgaggtgctgttcgagaagaggtacatgagcggcgccaaggacgtgagcgagatgttcaccaccaagttcgagaaggacaacttctacatcgagctgagccagggcaacaacctgtacggcggcccgatcgtgcacttctacgacgtgagcatcaagtaggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgacctcgaggcctaggcttaaggtttaaacagcccgggcgcgccggtaccgagctcgaattcggtaacccggtccgggccattctggccgtaccgagctcgaattcggcccaacttttctatacaaagttgatagcgataaatcctgaggatctggtcttcctaaggacccgggatatcggaccgattaaactttaattcggtccgataacttcgtatagcatacattatacgaagttatacctggtggcgccgctagcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatcaattcgctagcgaagttcctattccgaagttcctattctctagaaagtataggaacttcagatccaccgggatccacacgacaccatgtcccccgagcgccgccccgtcgagatccgcccggccaccgccgccgacatggccgccgtgtgcgacatcgtgaaccactacatcgagacctccaccgtgaacttccgcaccgagccgcagaccccgcaggagtggatcgacgacctggagcgcctccaggaccgctacccgtggctcgtggccgaggtggagggcgtggtggccggcatcgcctacgccggcccgtggaaggcccgcaacgcctacgactggaccgtggagtccaccgtgtacgtgtcccaccgccaccagcgcctcggcctcggctccaccctctacacccacctcctcaagagcatggaggcccagggcttcaagtccgtggtggccgtgatcggcctcccgaacgacccgtccgtgcgcctccacgaggccctcggctacaccgcccgcggcaccctccgcgccgccggctacaagcacggcggctggcacgacgtcggcttctggcagcgcgacttcgagctgccggccccgccgcgcccggtgcgcccggtgacgcagatctgagtcgaaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccctagcgtatacgaagttcctattccgaagttcctattctccagaaagtataggaacttctgtacacctgagctgattccgatgacttcgtaggttcctagctcaagccgctcgtgtccaagcgtcacttacgattagctaatgattacggcatctaggaccgactagctaactaactagtacgtagaattaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttaaaagatgaaaattaatgagacaatttttgttgaaacttatctaaaataaagcaaatatttgactaaatattgtgtgcgaaacatatccaaaataaataaaatatttcatgttaatttggactaaacatttccttactaaatccttaatccataaaggttaaaccctaaacatttgactaaacatttccctactaaatccttaatccataaatgtaacactctaaaattctattatgagatttagggaaattcttccaaaagttttggattaaaatatatttttataaaaaatctctgaaatagataaatggtgttagtattttttttgtaatataaaagttatacttaaagcatttaaatgggaaatgttatttgaaactccaaaagaaataaaacagggtcatctttaattggtaattgatgaaatacgcgtttagaaaccttctataaagaagatttaaaacaaaattaattcttaataaaagttagacaatataagctcttaaattaaataaataataaatacctaatttttaaatgaacttttaactagactccacattcaaattgaaattttattaagatgaatgacgtttgtgtattgca

SEQ ID NO: 15 - DNA, 421 bp, Artificial Sequence
  5′ flanking
ttctctggagtttcattatttcattgtacatatgaaaagaaacttacatcatataggagttttccaaaaatatgtatataatattgttgtataaaacataaaagcattattaaaccttgcagaggagcttccacatgattccaactgcatatttgattgaatttttcaagtcatctaaaaagaacaacatttaaagcaaaaactcgagtcaaattgatgtaacattagggattaccagatcccaatgcacgacgattgacacggtcgttggtgaggagtgacaaggtcagtggcttggggataggtgcgcttggttgatgtcagataattgagttcggcagctatgtgaggatcagtattgaggcacacaacctatggcggagcaagaacccgaagaaactcattctatttagtattgaga

SEQ ID NO: 16 - DNA, 606 bp, Artificial Sequence
  3′ flanking
aaagatgaaaattaatgagacaatttttgttgaaacttatctaaaataaagcaaatatttgactaaatattgtgtgcgaaacatatccaaaataaataaaatatttcatgttaatttggactaaacatttccttactaaatccttaatccataaaggttaaaccctaaacatttgactaaacatttccctactaaatccttaatccataaatgtaacactctaaaattctattatgagatttagggaaattcttccaaaagttttggattaaaatatatttttataaaaaatctctgaaatagataaatggtgttagtattttttttgtaatataaaagttatacttaaagcatttaaatgggaaatgttatttgaaactccaaaagaaataaaacagggtcatctttaattggtaattgatgaaatacgcgtttagaaaccttctataaagaagatttaaaacaaaattaattcttaataaaagttagacaatataagctcttaaattaaataaataataaatacctaatttttaaatgaacttttaactagactccacattcaaattgaaattttattaagatgaatgacgtttgtgtattgca

SEQ ID NO: 17 - protein, 694 aa, Artificial sequence
  variant
MAATTLTSALPGAFSSSQRPSAPFNLQRSPRVLRRFNRKTGRQPRGLVRAAKAQRSGTRSMGNSVLNSGRTTICDAYNVAAHDPFSFQHKSLDTVQREWTEWKKNNHSLYLDPIVGTVASFLLKKVGSLVGKRILSELRNLIFPSGSTNLMQDILRETEQFLNQRLDTDTLARVNAELTGLQANVEEFNRQVDNFLNPNRNAVPLSITSSVNTMQQLFLNRLPQFQMQGYQLLLLPLFAQAANLHLSFIRDVILNADEWGISAATLRTYRDYLKNYTRDYSNYCINTYQSAFKGLNTRLHGTLEFRTYMFLNVFEYVSIWSLFKYQSLLVSSGANLYASGSGPQQTQSFTSQDWPFLYSLFQVNSNYVLNGFSGARLSNTFPNIGGLPGSTTTHALLAARVNYSGGISSGDIGASPFNQNFNCSTFLPPLLTPFVRSWLDSGSDREGVATVTNWQTESFETTLGLRSGAFTARGNSNYFPDYFIRNISGVPLVVRNEDLRRPLHYNEIRNIASPSGTPGGARAYMVSVHNRKNNIHAVHENGSMIHLAPNDYTGFTISPIHATQVNNQTRTFISEKFGNQGDSLRFEQNNTTARYTLRGNGNSYNLYLRVSSIGNSTIRVTINGRVYTATNVNTTTNNDGVNDNGARFSDINIGNVVASSNSDVPLDINVTFNSGTQFDLMNTMLVPTNISPLY

SEQ ID NO: 18 - protein, 1182 aa, Artificial sequence
  variant
MGHNNPNINECIPYNCLSNPEVEVLGGERIETGYTPIDISLSLTQFLLSEFVPGAGFVLGLVDVIWGIFGPSQWDAFLVQIEQLINQRIEEFARNQAISRVEGLSNLYQIYAESFREWEADPTNPALKEEMRTQFNDMNSALTTAIPLFAVQNYQVPLLSVYVQAANLHLSVLRDVSVFGQRWGFDAATINSRYNDLTRLIGNYTDHAVRWHNTGLERIWGPDSRDWIRYNQFRRELTLTVLDIVSLFPNYDSRTYPIRTASQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNTIDPERINQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDRIEFVPAEVTFEAEYDLERAQKVVNALFTSSNQIGLKTDVTDYHIDQVSNLVDCLSDEFCLDEKRELSEKVKHAKRLSDERNLLQDPNFRGINRQPDRGWRGSTDITIQGGDDVFKENYVTLPGTVDECYPTYLYQKIDESKLKAYTRYELRGYIEDSQDLEIYLIRYNAKHEIVNVPGTGSLWPLSAQSPIGKCGEPNRCAPHLEWNPDLDCSCRDGEKCAHHSHHFTLDIDVGCTDLNEDLGVWVIFKIKTQDGHARLGNLEFLEEKPLLGEALARVKRAEKKWRDKREKLQLETNIVYKEAKESVDALFVNSQYDRLQVDTNIAMIHAADKRVHRIREAYLPELSVIPGVNAAIFEELEGRIFTAYSLYDARNVIKNGDFNNGLLCWNVKGHVDVEEQNNHRSVLVIPEWEAEVSQEVRVCPGRGYILRVTAYKEGYGEGCVTIHEIEDNTDELKFSNCVEEEVYPNNTVTCNNYTGTQEEYEGTYTSRNQGYDEAYGNNPSVPADYASVYEEKSYTDGRRENPCESNRGYGDYTPLPAGYVTKDLEYFPETDKVWIEIGETEGTFIVDSVELLLMEE

SEQ ID NO: 19 - protein, 789 aa, Bacillus thuringiensis
MNKNNTKLSTRALPSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 20 - protein, 183 aa, Streptomyces viridochromogenes
MSPERRPVEIRPATAADMAAVCDIVNHYIETSTVNFRTEPQTPQEWIDDLERLQDRYPWLVAEVEGVVAGIAYAGPWKARNAYDWTVESTVYVSHRHQRLGLGSTLYTHLLKSMEAQGFKSVVAVIGLPNDPSVRLHEALGYTARGTLRAAGYKHGGWHDVGFWQRDFELPAPPRPVRPVTQI

SEQ ID NO: 21 - DNA, 22 bp, Artificial Sequence
  primer
tcgagagattggattcggtaca

SEQ ID NO: 22 - DNA, 19 bp, Artificial Sequence
  primer
gggaacagcgacacgatgt

SEQ ID NO: 23 - DNA, 15 bp, Artificial Sequence
  probe
cgagctgaccctcac

SEQ ID NO: 24 - DNA, 21 bp, Artificial Sequence
  primer
cgcactttcatcagcgagaag

SEQ ID NO: 25 - DNA, 24 bp, Artificial Sequence
  primer
tgttctgctcaaacctcagagaat

SEQ ID NO: 26 - DNA, 14 bp, Artificial Sequence
  probe
tcggcaaccaaggc

SEQ ID NO: 27 - DNA, 24 bp, Artificial Sequence
  primer
accagagcgagcaaatctactaca

SEQ ID NO: 28 - DNA, 21 bp, Artificial Sequence
  primer
tagcgcagggtcttcatcttc

SEQ ID NO: 29 - DNA, 18 bp, Artificial Sequence
  probe
cgtgttcccgaacgagta

SEQ ID NO: 30 - DNA, 24 bp, Artificial Sequence
  primer
catcgtgaaccactacatcgagac

SEQ ID NO: 31 - DNA, 18 bp, Artificial Sequence
  primer
gtcgatccactcctgcgg

SEQ ID NO: 32 - DNA, 22 bp, Artificial Sequence
  probe
accgtgaacttccgcaccgagc